(19) United States
(12) Patent Application Publication
(10) Pub. No.: US 2011/0236431 A1
(43) Pub. Date: Sep. 29, 2011

(54) TREATMENT OF ACUTE MYOCARDIAL INFARCTION (AMI) USING ENCAPSULATED CELLS ENCODING AND SECRETING GLP-1 PEPTIDES OR ANALOGS THEREOF

(75) Inventors: Christine Wallrapp, Grossostheim (DE); Andrew Lennard Lewis, Surrey (GB); Peter William Stratford, Surrey (GB); Eric Thoenes, Budingen (DE)

(73) Assignee: BIOCOMPATIBLES UK LTD., Farnham, Surrey (GB)

(21) Appl. No.: 13/061,404

(22) PCT Filed: Sep. 11, 2009
(86) PCT No.: PCT/EP2009/006620

§ 371 Date: May 23, 2011

(30) Foreign Application Priority Data
Sep. 12, 2008 (EP) 08016137.5
Sep. 11, 2009 (EP) PCT/EP2009/006620

(51) Int. Cl. A61K 9/00; A61P 9/10; A61K 35/12

(52) U.S. Cl. 424/400; 424/93.21; 424/93.7

(57) ABSTRACT

The present application refers to the use of cells, e.g. mesenchymal stem cells or mesenchymal stromal cells, or any further suitable cell, encoding and secreting GLP-1, a fragment or variant thereof or a fusion peptide comprising GLP-1 or a fragment or variant thereof, for the treatment of acute myocardial infarction (AMI or Ml), wherein the cells, encoding and secreting GLP-1, a fragment or variant thereof or a fusion peptide comprising GLP-1 or a fragment or variant thereof, are encapsulated in a (spherical) microcapsule to prevent a response of the immune system of the patient to be treated. The present application also refers to the use of these (spherical) microcapsule(s) or of a pharmaceutical composition containing these cells or (spherical) microcapsule(s) for the treatment of acute myocardial infarction (AMI or Ml).

The present application refers to the use of cells, e.g. mesenchymal stem cells or mesenchymal stromal cells, or any further suitable cell, encoding and secreting GLP-1, a fragment or variant thereof or a fusion peptide comprising GLP-1 or a fragment or variant thereof, for the treatment of acute myocardial infarction (AMI or MI), wherein the cells, encoding and secreting GLP-1, a fragment or variant thereof or a fusion peptide comprising GLP-1 or a fragment or variant thereof, are encapsulated in a (spherical) microcapsule to prevent a response of the immune system of the patient to be treated. The present application also refers to the use of these (spherical) microcapsule(s) or of a pharmaceutical composition containing these cells or (spherical) microcapsule(s) for the treatment of acute myocardial infarction (AMI or MI).

Acute myocardial infarction (AMI or MI), more commonly known as a heart attack, is a common medical condition that occurs when the blood supply to a part of the heart is interrupted, most typically due to rupture of a vulnerable plaque. The resulting ischemia or oxygen shortage causes damage and potential death of heart tissue. AMI is a medical emergency, and the leading cause of death for both men and women all over the world (see e.g. The World Health Report 2004—Changing History (PDF), World Health Organization, 120-4, ISBN 92-4-156265-X). Important risk factors are a previous history of vascular disease such as atherosclerotic coronary heart disease and/or angina, a previous heart attack or stroke, any previous episodes of abnormal heart rhythms or syncope, older age-especially men over 40 and women over 50, smoking, excessive alcohol consumption, the abuse of certain drugs, high triglyceride levels, high LDL (“low-density lipoprotein”) and low HDL (“high density lipoprotein”), diabetes, high blood pressure, obesity, and chronically high levels of stress in certain persons.

Myocardial infarction is a common presentation of ischemic heart disease. The WHO estimated that in 2002, 12.6 percent of deaths worldwide are due to ischemic heart disease (see, e.g., The World Health Report 2004, supra). Ischemic heart disease is the leading cause of death in developed countries, but third to AIDS and lower respiratory infections in developing countries (see e.g. Cause of Death—UC Atlas of Global Inequality, Center for Global, International and Regional Studies (CGIRS) at the University of California Santa Cruz, retrieved on Dec. 7, 2006).

In the United States, diseases of the heart are the leading cause of death, causing even a higher mortality than cancer (malignant neoplasms) (see e.g. “Deaths and percentage of total death for the 10 leading causes of death: United States”: 2002-2003, National Center of Health Statistics, retrieved on Apr. 17, 2007). Coronary heart disease is responsible for 1 in 5 deaths in the U.S. Some 7,200,000 men and 6,000,000 women are living with some form of coronary heart disease. 1,200,000 people suffer a (new or recurrent) coronary attack every year, and about 40% of them die as a result of the attack (see e.g. Heart Attack and Angina Statistics, American Heart Association (2003), retrieved on Dec. 7, 2006). This means that about every 65 seconds, an American dies of a coronary event. Similar statistics are to be expected in Europe.

Immediate treatment for suspected acute myocardial infarction typically includes oxygen, aspirin, glyceryl trinitrate and pain relief, usually morphine sulfate. The patient normally receives a number of diagnostic tests, such as an electrocardiogram (ECG, EKG), a chest X-ray and blood tests to detect elevated creatine kinase or troponin levels (which are chemical markers released by damaged tissues, especially the myocardium). Further treatment may include either medications to break down blood clots that block the blood flow to the heart, or mechanically restoring the flow by dilatation or bypass surgery of the blocked coronary artery. Coronary care unit admission allows rapid and safe treatment of complications such as abnormal heart rhythms.

AMI is a type of acute coronary syndrome, which is most frequently (but not always) a manifestation of coronary artery disease. The most common triggering event is the disruption of an atherosclerotic plaque in an epicardial coronary artery, which leads to a clotting cascade, sometimes resulting in total occlusion of the artery. Atherosclerosis is the gradual build-up of cholesterol and fibrous tissue in plaques in the wall of arteries (in this case, the coronary arteries), typically over decades. Blood stream column irregularities visible on angiographies reflect artery lumen narrowing as a result of decades of advancing atherosclerosis. Plaques can become unstable, rupture, and additionally promote a thrombus (blood clot) that occludes the artery, typically within minutes. When a severe plaque rupture occurs in the coronary vasculature, it may lead to myocardial infarction and usually to a necrosis of downstream myocardium.

If impaired blood flow to the heart lasts long enough, it triggers a process called the ischemic cascade, in which the heart cells typically die (particularly due to necrosis) and do not grow back. A collagen scar forms in its place. Recent studies indicate that apoptosis may also play a role in the process of tissue damage subsequent to myocardial infarction (see Krijnen P A, Nijmeijer R, Meijer C J, Visser C A, Hack C E, Niessen H W. (2002). “Apoptosis in myocardial ischaemia and infarction”. J Clin Pathol 55 (11): 801-11. PMID 12401816). As a result, the patient's heart can be permanently damaged. The scar tissue formed in the ischemic cascade also puts the patient at risk for potentially life threatening arrhythmias. A therapy, which reduces the extent of this ensuing tissue damage, could have significant benefits in improving the function of the heart post AMI and improved long term patient outcomes.

At present, there is a deal of great research in the use of stem cell therapy for the regeneration of the myocardium post MI. Patients who receive stem cell treatment by left ventricular intramyocardial implantation of stem cells derived from their own bone marrow after a myocardial infarction show improvements in left ventricular ejection fraction and end-diastolic volume, which was not seen with placebo. The larger the initial infarct size, the greater the effect of the infusion. However, a suitable therapy has not yet been established. Clinical trials of progenitor cell infusion as a treatment approach to ST elevation MI are proceeding (see e.g. Schachinger V, Erbs S, Elsasser A, Haberbosch W, Hambrecht R, Holschermann H, Yu J, Corti R, Mathey D G, Hamm C W, Suselbeck T, Assmus B, Tonn T, Dimmeler S, Zeiher A M; REPAIR-AMI Investigators (2006), “Intracoronary bone marrow-derived progenitor cells in acute myocardial infarction”, N Engl J Med 355 (12): 1210-21, PMID 16990384.).

Additional to the herein stem cell approach, there are currently at least three biomaterial and tissue engineering approaches for the treatment of AMI or MI. However, these approaches are in an even earlier stage of medical research, so many questions and issues need to be addressed prior to establishing a suitable therapy based on these approaches.

The first interesting approach in this field involves polymeric left ventricular restraints in the prevention of heart failure. A second approach utilizes in vitro engineered cardiac tissue, which is subsequently implanted in vivo.

Another approach entails injecting cells and/or a scaffold into the myocardium to create in situ engineered cardiac tissue (see e.g. Christman K L, Lee R J. “Biomaterials for the Treatment of Myocardial Infarction”. J Am Coll Cardiol 2006; 48(5): 907-13. PMJD 16949479). A variant of this approach is the injection of cells to produce factors that will help preserve myocardium post AMI or MI, for instance, by preventing the cardiomyocytes from undergoing apoptosis. In this final approach, Glucagon Like Peptide-1 (GLP-1) has been shown to provide some very promising effects, which may be utilized in the treatment of AMI or MI. A number of recent studies, carried out in this approach, have shown that Glucagon Like Peptide-1 (GLP-1), one of the most potent incretin hormones, has potential beneficial actions on the ischaemic and failing heart (see Bose A K, Mocanu M M, Carr R D, Yellon D M, Myocardial ischaemia-reperfusion injury is attenuated by intact glucagon like peptide-1 (GLP-1) in the in vitro rat heart and may involve the p7Os6K pathway. Cardiovasc Drugs Ther. 2007 August; 21(4):253-6; and Nikolaidis L A, Mankad S, Sokos G G, Miske G, Shah A, Elahi D, Shannon R P. Effects of glucagon-like peptide-1 in patients with acute myocardial infarction and left ventricular dysfunction after successful reperfusion, Circulation, 2004 Mar. 2, 109(8):962-5).

GLP-1 is located on the glucagon gene, a well studied gene (see e.g. White, J. W. et al., 1986 Nucleic Acid Res. 14(12) 4719-4730). The preproglucagon molecule as a high molecular weight precursor molecule is synthesized in pancreatic alpha cells and in the jejunum and colon L cells. Preproglucagon is a 180 amino acid long prohormone and its sequence contains, in addition to glucagon, two sequences of related structure: glucagon-like peptide-1 (GLP-1) and glucagon-like peptide-2 (GLP-2). In the preproglucagon molecule, between GLP-1 and GLP-2 is a 17 amino acid peptide sequence (or rather a 15 amino acid sequence plus the C-terminal RR cleavage site), intervening peptide 2 (IP2). The IP2 sequence (located between GLP-1 and GLP-2 in the precursor molecule) is normally cleaved proteolytically after aa 37 of GLP-1. The preproglucagon module is therefore cleaved into various peptides, depending on the cell, and the environment, including GLP-1 (1-37), a 37 amino acid peptide in its unprocessed form. Generally, this processing occurs in the pancreas and the intestine. The GLP-1 (1-37) sequence can be further proteolytically processed into active GLP-1 (7-37), the 31 amino acid processed form, or GLP-1 (7-36) amide. Accordingly, the designation GLP-1(7-37) means that the fragment in question comprises the amino acid residues from (and including) number 7 to (and including) number 37 when counted from the N-terminal end of the parent peptide, GLP-1.

GLP-1 is a gut hormone and is the most potent endogenous insulinotropic agent with actions that include stimulating adenylate cyclase and protein kinase activity in the beta-cell. Physiologically, together with gastric inhibitory polypeptide from the upper gut, it functions as an incretin hormone lowering the blood glucose level. Accordingly, GLP-1, secreted in response to food intake, has multiple effects on, e.g., the stomach, liver, pancreas and brain that work in concert to regulate blood sugar. Consequently, Glucagon-like peptide GLP-1(7-36)amide, and its non-amidated analogue GLP-1(7-37) have attracted considerable interest because of their potent actions on carbohydrate metabolism and its potential applicability to the treatment of diabetes, including type 2 diabetes.

Additionally, GLP-1 has shown promising effects on the treatment of acute myocardial infarction (AMI) or myocardial infarction (MI). As mentioned herein, GLP-1 is a naturally occurring incretin with both insulinotropic and insulinomimetic properties that stimulate glucose uptake without the requirements for concomitant glucose infusion. Nikolaidis et al. (see Nikolaidis et al., 2004, supra) found that when added to standard therapy, GLP-1 infusion improved regional and global LV function in patients with AMI and severe systolic dysfunction after successful primary angioplasty.

In an early study, the safety and efficacy of the administration of GLP-1 was tested during a 72 hour infusion of GLP-1 (1.5 μg/kg per minute) added to a background therapy in 10 patients with AMI and LV fraction (EF)<40% after successful primary angioplasty compared to 11 control patients was determined (see Nikolaidis et al., Circulation 2004, 109; 962-965). In the context of these experiments, the benefits of GLP-1 were regarded to be independent of AMI location or history of diabetes. Accordingly, GLP-1 infusion, when added to standard therapy, seems to improve regional and global LV function in patients with AMI and severe systolic dysfunction after successful primary angioplasty (see Nikolaidis et al., Circulation 2004, supra). Nikolaidis et al. also hypothesized that GLP-1 facilitates recovery from myocardial stunning after an ischemic event. In an experimental dog model, GLP-1 was administered upon a 10 minute occlusion of the left ventricular coronary artery, followed by a 24 hour reperfusion. In these experiments, administration of GLP-1 caused an insulinotropic effect, but no hyperglycaemia (see Nikolaidis et al., Journal of Pharmacology and Experimental Therapeutics, January 2005, 312 (1), pp. 303-8).

According to another approach, the role of GLP-1 in vivo, particularly GLP-1 (7-36), was investigated by using DPP-IV inhibitors such as valine pyrrolidine (VP) as a means of preventing its degradation (see Bose et al., Diabetes, Vol. 54, January 2005, and Bose et al, Cardiovascular Drugs and Therapy, August 2007, 21 (4), pp. 253-6).

Native GLP-1, particularly GLP-1 (7-36), suffers from a short half life in vivo. It is rapidly degraded in plasma within minutes by DPP-IV between residues 8 and 9, resulting in an inactive NH₂-terminally truncated metobolite GLP-1 (9-36). In this context, GLP-1 and VP were seen to protect the myocardium from ischemia reperfusion injury in the in vivo heart model, demonstrating a significant reduction in infarction compared to VP or saline groups. In the corresponding in vitro study, those hearts treated with GLP-1 and VP again demonstrated a significantly reduced infarct size compared with control and VP groups (see also Bose et al., 2005 and 2007, supra).

Further data suggested that GLP-1 can exert a direct protective effect to the heart via inhibition of apoptosis either directly in target cells expressing GLP-1 receptor or possibly via activation of survival factors such as prosurvival signalling pathways (see also Bose et al, Diabetes, Vol. 54, January 2005). In this context, the main target of GLP-1 is the islet, where the hormone stimulates the insulin secretion, promotes beta cell proliferation and neogenesis, and inhibits glucagon secretion. However, GLP-1 receptors are also expressed outside the islets, confirming the likelihood that GLP-1 also plays a role in other organs (see Ahren B. et al, Hormones et metabolisme, December 2004, 36 (11-12), pp. 842-5).

GLP-1 has been shown to increase left ventricular (LV) function, myocardial glucose uptake, and GLUT-1 and GLUT-4 translocation during reperfusion to an extent similar to that with insulin. In contrast to insulin, however, the insulinotropic actions of GLP-1 are advantageously dependent on the ambient glucose concentrations, mitigating the risks of hypoglycaemia. GLP-1 has also been shown to have direct effects on the normal heart, reducing contractility but increasing myocardial glucose uptake through a non-Akt-1 dependent mechanism, distinct from the actions of insulin (see Tingcun Zhao et al., The Journal of Pharmacology and Experimental Therapeutics, Vol. 317(3), pp. 1106-1113; No. 3, 2006)

GLP-1 has furthermore been shown to improve left ventricular (LV) function in patients with acute myocardial infarction and left ventricular dysfunction. GLP-1 additionally promotes the activity of phospho-inositide 3-kinase (PI3K) in beta-cells. This kinase has been clearly associated with myocardial protection in the setting of ischemic/reperfusion injury as well as myocardial preconditioning (see Bose et al., Diabetes, Vol. 54, January 2005; and Tingcun Zhao et al., The Journal of Pharmacology and Experimental Therapeutics, Vol. 317, pp. 1106-1113; No. 3, 2006).

In a different approach, Huisamen et al. (see Huisamen et al., Cardiovascular Journal of Africa (South Africa), March-April 2008, 19(2), p. 77-83), showed that GLP-1 had an infarct-sparing effect when supported by the presence of the DPP-IV inhibitor VP. GLP-1 could not directly activate Akt (also called protein kinase B) or the extracellular regulated kinases Erk1/2 in hearts or cardiocytes under normoxic conditions, but phosphorylation of the AMP-activated kinase (AMPK) on Thr(172) was enhanced. In addition, the glycolytic enzyme phosphofructokinase-2 was activated dose dependently. During reperfusion after ischaemia, modulation of the phosphorylation of PKB/Akt as well as AMPK was evident. GLP-1 thus directly appears to protect the heart against low-flow ischaemia by enhancing glycolysis.

However, the use of native GLP-1 causes some problems in vivo due to its rapid degradation in vivo by DPP-IV as already described herein. Additionally, GLP-1 undergoes renal excretion. These factors raise the issue, as to which peptide, GLP-1 (7-36) or the NH₂-terminally truncated metabolite GLP-1 (9-36), is the active moiety in vivo and as to whether physiological effects are exerted in therapeutic applications by the native GLP-1 or its fragments.

Due to its rapid degradation in vivo, GLP-1 was regarded as a suitable tool only for a short-term metabolic control, such as intensive care units potentially useful in patients with acute myocardial infarction, coronary surgery, cerebrovascular events, or septicaemia. For more long-term metabolic control, incretin mimetics such as agonists of the GLP-1 receptor were suggested (see e.g. Nauck M. A., Nov-Hormones et metabolisme, December 2004, 36 (11-12), pp. 852-8).

However, irrespective of its fast degradation in vivo, GLP-1 appears to provide a good basis as a potential drug in the treatment of coronary diseases, particularly for the treatment of AMI or MI.

Various attempts have therefore been made to synthesize stabilized (against DPP-IV) analogues of naturally occurring GLP-1 (GLP-1(7-37)). In particular, the 8^{th} residue, which in vivo is Ala, was replaced by another residue, for instance, Gly, Ser or Thr (Burcelin, R. et al. (1999) Metabolism 48, 252-258). The Gly8 or G8 analogue has been extensively tested, both as synthesized molecule, and produced by cell lines genetically engineered to secrete the mutant polypeptide (Burcelin, R., et al. (1999), Annals of the New York Academy of Sciences 875: 277-285). Various other modifications have been introduced into GLP-1(7-37) to enhance its in vivo stability without compromising its biological activity. However, all of these approaches did not achieve any therapeutic significance due to considerable problems involved.

Nevertheless, none of these approaches allows a long-term provision of GLP-1 in vivo. This is due to proteolytical degradation as discussed herein, to metabolism of GLP-1 and normal protein degradation typically occurring in the body. Thus, at present, the patient in need of GLP-1 has to receive one or even multiple doses of GLP-1 or its analogs or variants during a long period of time, i.e. as long as he suffers from the disease to be treated, or even worse, for a whole life span. In this period of time, administration typically occurs within short intervals due to the short half life of GLP-1 in vivo. Furthermore, doses of GLP-1 have to be administered either by a medical doctor or by the patient himself. This represents a major challenge and burden for patient and environment. In order to circumvent this problem, GLP-1 may be administered by providing cells to a patient containing a nucleic acid encoding and expressing GLP-1. Implantation of such cells would ensure a longer provision of GLP-1 in vivo and, due to secretion of GLP-1 from the grafted cells, provide GLP-1 directly at the site of interest.

WO 99/53064 discloses a strategy for creating a multimeric GLP-1 expression cassette which can be incorporated into a variety of cell types which are publicly available immortalised cell lines and dividing primary cell cultures. Examples include EGF-responsive neurospheres, bFGF-responsive neural progenitor stem cells from the CNS of mammals, while the worked example uses baby hamster kidney (BHK) cells. The implanted transfected cells were said to have been used successfully to treat diabetic mice, allowing glucose control equivalent substantially to non-diabetic controls. However, this kind of implantation technique does not comply with the requirements for a routine treatment for e.g. diabetes patients or have been used successfully in AMI or MI patients.

Moreover, it is known in the art that the immune system typically recognizes foreign cells and triggers an immune response in order to protect the organism from such external material. Implantation of cells capable of expressing GLP-1 or any of its variants or derivatives may thus lead to an immune response in the organism. Such a defense response may cause considerable and undesirable side effects during treatment and may lead to severe complications or even death of the treated organism.

One strategy to circumvent this problem may be the use of autologous cells, i.e. cells which are derived from the patient itself, which are genetically altered to transiently express GLP-1, particularly GLP-1(7-36). Such a procedure, however, is typically limited to the cells of the specific patient and requires a long-term preparation in a laboratory prior to use. AMI or MI, however, requires a therapy within a few minutes or hours subsequent to the myocardial infarction, i.e. a short term provision of an efficient medicament.

In summary, at present there is no efficient AMI or MI therapy available in the art, which allows providing an efficient therapy directly subsequent to occurrence of the myocardial infarction in a patient to be treated. In other words, the prior art fails to provide a therapy which reflects the entire spectrum of beneficial effects known for GLP-1, e.g. its activity to powerfully reduce the damages caused by ischemia or oxygen shortage and potential death of heart tissue without the need of repeated administration of GLP-1 peptide(s) and/or the risk of an undesired immune response against e.g. implanted GLP-1 expressing allogenic cells.

Therefore, it is an objective of the present invention to provide an efficient therapy for AMI or MI using GLP-1 based peptide molecules or analogues thereof, which are biologically active in vivo over a long-lasting time period without the need of repeated administration of GLP-1 peptide(s) and/or the risk of evoking an undesired immune response.

The object underlying the present invention is solved by the attached claims, particularly by the use of cells, e.g. mesenchymal stem cells or mesenchymal stromal cells, or any further cell, that may be used in the context of the present invention, encoding and secreting GLP-1, a fragment or variant thereof or a fusion peptide comprising GLP-1 or a fragment or variant thereof, for the treatment of AMI or MI or diseases related thereto, wherein the cells, encoding and secreting GLP-1, a fragment or variant thereof or a fusion peptide comprising GLP-1 or a fragment or variant thereof, are encapsulated in a (spherical) microcapsule to prevent a response of the immune system of the patient to be treated. In the context of the present invention the term “cells encoding . . . ” typically means “cells, which are engineered to contain or comprise nucleic acids encoding . . . ”.

The cells used for providing the herein described inventive solution, encoding and secreting GLP-1, a fragment or variant thereof or a fusion peptide comprising a GLP-1 peptide or a fragment or variant thereof for the treatment of AMI or MI or diseases related thereto, are preferably encapsulated in a (spherical) microcapsule to prevent a response of the immune system of the patient to be treated. In the context of the present invention, such a (spherical) microcapsule preferably comprises a (spherical) core (i.e. the core may be spherical or not) and at least one surface coating layer, wherein:
the (spherical) core comprises or consists of (a mixture of) cross-linked polymers and cells, e.g. mesenchymal stem cells or mesenchymal stromal cells, or any further cell (type), that may be used in the invention, encoding and secreting GLP-1, a fragment or variant thereof or a fusion peptide comprising GLP-1 or a fragment or variant thereof, as defined herein; and
the at least one surface coating layer comprises or consists of (a mixture of) of typically cross-linked polymers.

The (spherical) microcapsule, comprising cells as used herein encoding and secreting GLP-1, a fragment or variant thereof or a fusion peptide comprising GLP-1 or a fragment or variant thereof, as defined herein, typically comprises a particle size, herein referred to as the total diameter of the (spherical) microcapsule. Generally, the total diameter of the (spherical) microcapsule as used herein may vary considerably depending on the specific treatment and administration mode. In the context of the present invention, the treatment typically occurs locally by administration of the (spherical) microcapsule as used herein into a specific administration site, e.g. by injection or implantation. Accordingly, the administration mode may limit the total diameter of the (spherical) microcapsule as used herein, e.g. by the diameter of the injection cannula. The total diameter of the (spherical) microcapsule as used herein is furthermore determined by the diameter of the core of the (spherical) microcapsule as well as by the thickness of the at least one surface coating layer(s), as both diameters typically depend at least in part on each other and of course, influence the total diameter of the (spherical) microcapsule.

For the treatment of AMI or MI diseases as defined herein and diseases related thereto, the inventors of the present application have surprisingly found, that a total diameter (particle size) of the (spherical) microcapsule of about 120 μm to about 800 μm, preferably of about 120 μm to about 700 μm, more preferably a total diameter of about 150 μm to about 650 μm, and even more preferably a total diameter of about 165 μm to about 600 μm may be used. Particularly, a total diameter (particle size) of the (spherical) microcapsule of about 120 μm to about 300 μm, more preferably a total diameter of about 150 μm to about 250 μm, even more preferably a total diameter of about 165 μm to about 225 μm, and most preferably a total diameter of about 180 μm to about 200 μm, e.g. about 180, 185, 190 or 200 μm, is advantageous for the treatment of AMI or MI. (Spherical) microcapsules, comprising such a total diameter, are typically retained in the myocardium in the selected site of injection and do not migrate into the surrounding tissue. This allows providing a continuous expression of GLP-1 and/or its variants or analogs at the site of injection during treatment for a sufficient period of time to provide the entire spectrum of beneficial effects known for GLP-1, e.g. its activity to powerfully reduce the damages caused by ischemia or oxygen shortage and potential death of heart tissue.

In the herein context, the term “spherical” is understood in its broadest meaning. A spherical particle is preferably understood to have a sphere-like shape, whereby the shape may be symmetrical or asymmetrical, e.g. a (spherical) microcapsule and/or its core may have ellipsoidal shape. In a less preferred embodiment the microcapsule or core used according to the present invention may not be spherical within the herein meaning, but may have an arbitrary shape with e.g. protruding or invading segments on the surface of the microcapsule. Wherever in the present disclosure “spherical” microcapsules or cores are mentioned, “non-spherical” microcapsules or cores may be provided, prepared or used as well.

The (spherical) microcapsule as defined herein preferably comprises a (spherical) core (i.e. the core may be spherical or not), wherein the (spherical) core comprises or consists of (a mixture of) cross-linked polymers and cells, e.g. mesenchymal stem cells or mesenchymal stromal cells, or any other cell (type), that may be used in the context of the present invention, encoding and secreting GLP-1, a fragment or variant thereof or a fusion peptide comprising GLP-1 or a fragment or variant thereof for treatment of AMI or MI diseases, as defined herein, or diseases related thereto.

In the context of the present invention, the typically cross-linked polymers of the (spherical) core of the (spherical) microcapsule form a scaffold structure embedding the cells, e.g. mesenchymal stem cells or mesenchymal stromal cells, or any other cell (type), that may be used in the context of the present invention, in its cavities. These cells may be embedded in the scaffold structure individually or, typically, as aggregates, e.g. as (a pool of) aggregated cells of about 10 to about 10,000 cells, e.g. about 10 to about 500, about 10 to about 1,000 or about 10 to about 10,000 cells, more preferably 10 to about 100 or 10 to about 1000 cells. Preferably, the (spherical) core comprises a homogenous distribution of the cross-linked polymers and of embedded cells as defined herein. Preferably, the core, including the scaffold structure and the embedded cells as defined herein, is prepared according to a method as disclosed below. In this context, it is of critical importance to embed the encapsulated cells of the (spherical) microcapsule, e.g. mesenchymal stem cells or mesenchymal stromal cells, autologous cells or any other cell (type), which may be used in the context of the present invention, entirely in the polymer matrix when preparing (spherical) microcapsules for the use according to the present invention.

The embedded cells, e.g. mesenchymal stem cells or mesenchymal stromal cells, or any other cell (type) as defined herein, that may be used for the (spherical) microcapsule in the context of the present invention, may be present in the core in a concentration of about 1×10⁵, about 1×10⁶ or about 1×10⁷ cells/ml cross-linked scaffold polymer to about 5×10⁸ cells/ml cross-linked scaffold polymer, more preferably in a concentration of about 1×10⁵, about 1×10⁶, or about 1×10⁷ cells/ml cross-linked scaffold polymer to 1×10⁸ cells/ml cross-linked scaffold polymer and most preferably in a concentration of about 1×10⁵, about 1×10⁶, or about 2×10⁷ cells/ml cross-linked scaffold polymer to 6×10⁷ cells/ml cross-linked scaffold polymer.

The cells embedded in the (spherical) core of the (spherical) microcapsule is typically dependent on the diameter of the (spherical) core as defined above. As an example, an exemplary inventive (spherical) microcapsules having a total diameter of about 160 μm may comprise in its (spherical) core a number of embedded cells, e.g. mesenchymal stem cells or mesenchymal stromal cells, or any other cell (type) as defined herein, e.g. of about e.g. 10 to 100, preferably of about 30 to 80, e.g. about 60 to 70 cells per (spherical) core and thus per (spherical) microcapsule. Accordingly, administration of about 60,000 inventive (spherical) microcapsules typically provides about 3 to 4 million cells at once into the site to be treated.

The core of the (spherical) microcapsule used according to the present invention typically has a diameter (particle size) of not more than the diameter of the total diameter of the (spherical) microcapsule as defined herein. Typically, the core of the (spherical) microcapsule used according to the present invention has a diameter of about 50 μm to about 220 μm, preferably a diameter of about 100 μm to about 200 μm, likewise preferably a diameter of about 115 μm to about 185 μm, more preferably a diameter of about 130 μm to about 170 μm, and even more preferably a diameter of about 145 μm to about 155 μm, e.g. about 120, 125, 130, 135, 140, 145, 150 or 155 μm. Particularly preferred, the core of the (spherical) microcapsule, as used according to the present invention, has a diameter, which is preferably about 1 to about 80 μm less than the total diameter of the (spherical) microcapsule as defined herein, more preferably about 15 to about 70 μm less than the total diameter of the (spherical) microcapsule as defined herein, and most preferably about 30 to about 60 μm less than the total diameter of the (spherical) microcapsule as defined herein. In other words, the diameter of the core of the (spherical) microcapsule, as used according to the present invention, may have a size of about 50 μm, of about 60 μm, of about 70 μm, of about 80 μm, of about 90 μm, of about 100 μm, of about 110 μm, of about 120 μm, of about 125 μm, of about 130 μm, of about 135 μm, of about 140 μm, of about 145 μm, of about 150 μm, of about 155 μm, of about 160 μm, of about 165 μm, of about 170 μm, of about 175 μm, of about 180 μm, of about 185 μm, of about 190 μm, of about 195 μm, of about 200 μm, of about 205 μm, of about 210 μm, of about 215 μm, or even of about 220 μm, or may comprise any range selected from any two of the herein mentioned specific values.

The core of the (spherical) microcapsule as defined herein comprises cells, encoding and secreting GLP-1, a fragment or variant thereof or a fusion peptide comprising GLP-1 or a fragment or variant thereof, as defined herein, for treatment of AMI or MI diseases, as defined herein, or diseases related thereto. Such cells, e.g. mesenchymal stem cells or mesenchymal stromal cells, or any other cell (type), that may be used in the context of the present invention for the (spherical) core, being located at the core periphery or cells protruding out of the scaffold structure may evoke immunological problems, since the immune system will recognize these microcapsules as foreign components and, thus, these microcapsules will be attacked by the immune system.

Although this effect may be avoided by lowering the cell concentration in the initial solution, the present invention allows improving the efficacy of the microcapsule by increasing the core's cell portion. The higher the concentration of cells in the core, the smaller the total volume of the resultant microcapsules to be transplanted, i.e. the more efficient the microcapsules may work at the site of injection. In order to avoid immunological problems when using high concentrations of cells in the (spherical) core of the (spherical) microcapsule, the invention provides at least one surface coating layer applied on the (spherical) core. This surface coating layer does not allow an immune response to occur, even if cells are located very closely to the core periphery, since these cells are not accessible for the host's immune system due to the surface coating layer acting as barrier. This surface coating layer is typically composed (of a mixture) of a typically cross-linked polymer as defined herein without containing any cells. According to a particular preferred embodiment the afore defined (spherical) core is coated with at least one or more than one surface coating layer(s), e.g. 1, 2, 3, 4, 5, 5-10 or more surface coating layer(s), more preferably 1, 2 or 3 surface coating layer(s), most preferably with only one surface coating layer. Typically, each surface coating layer comprises a uniform thickness around the core. The thickness of the surface coating layer(s) of the (spherical) microcapsule, as used according to the present invention, may be varied almost arbitrarily and is typically in a range of about 1 to about 80 μm, more preferably in a range of about 15 to about 70 μm, and most preferably in a range of about 20 to about 40 μm, e.g. about 30 μm.

The (spherical) core of the (spherical) microcapsule as used herein (and optionally of the at least one surface coating of the (spherical) microcapsule) comprises or consists of (a mixture of) cross-linked polymers. In this context, any pharmaceutically acceptable (cross-linkable) polymer known in the art and being suitable for encapsulation may be used for the formation of the (spherical) core and, independent from each other, the at least one surface coating layer(s) of the (spherical) microcapsule, as defined according to the present invention. Preferably, such polymers are used, which, on the one hand, are permeable in their cross-linked state for supply of oxygen and nutrients from outside, and, on the other hand, allow diffusion of the peptide(s) encoded and secreted by the core cells from the microcapsule into the patient's tissue or body fluids. Furthermore, the cross-linked polymers prevent intrusion of components of the body's immune system through the matrix. By way of example, polymers may be used such as synthetic, semi-synthetic and natural water-soluble (bio)polymers, e.g. from natural polymers such as selected proteins or polymers based on proteins (e.g. collagens, albumins etc.), polyamino acids (e.g. poly-L-lysine, poly-L-glutamic acid, etc.), polysaccharides and their derivatives (e.g. carboxylmethyl cellulose, cellulose sulfate, agarose, alginates including alginates of brown algae (e.g. of species Laminarales, Ectocarpales, Fucales), carrageenans, hyaluronic acid, heparin and related glycosamino sulfates, dextranes and its derivatives, chitosan and their derivatives). Synthetic polymers may also be used such as e.g. aliphatic polyesters (e.g. polylactic acid, polyglycolic acid, polyhydroxybutyrates, etc.), polyamides, polyanhydrides, polyorthoesters, polyphosphazenes, thermoplastic polyurethanes, polyvinyl alcohols, polyhydroxyethylmethacrylates, polymethylmethacrylates and polytetrafluoroethylenes, etc.

Furthermore, block polymers may be used herein accordingly, i.e. polymers derived by combination of two or more of the aforementioned polymers. Such block polymers may be selected by a skilled person depending on the desired properties, e.g. pore size, cross-linking status, toxicity, handling, biocompatibility, etc. Any of the herein polymers is defined as a “chemically different polymer” in the context of the present invention, i.e. each of these polymers typically does not exhibit an identical molar mass and structure with any other of the herein polymers. In contrast, “chemically identical polymers” means, that the polymers exhibit an identical molar mass and structure.

Finally, mixtures of the herein polymers are also encompassed herein, wherein the amounts of polymers contained in such a mixture may be selected by a skilled person depending on the desired properties, e.g. as outlined herein. In this respect, mixtures of polymers may be regarded as chemically identical to another polymer mixture (“chemically identical polymers”), if the overall molar mass of the resultant polymer mixture and the corresponding molar percentage of the single polymers of the mixture are identical to the other polymer mixture.

Preferably, the (mixture of) cross-linked polymers of the (spherical) core of the (spherical) microcapsule as used herein (and optionally of the at least one surface coating layer of the (spherical) microcapsule) comprise or consist of alginate(s). Alginates, if used according to present invention as a polymer for the formation of the (spherical) core and/or of the at least one surface coating layer are particularly advantageous due to their biocompatibility and cross-linking properties. From a chemical point of view, alginates are anionic polysaccharides derived from homopolymeric groups of β-D-mannuronic acid and α-L-guluronic acid, separated by heteropolymeric regions of both acids. Alginates are water soluble and form high viscosity solutions in the presence of monovalent cations such as sodium or potassium. A cross-linked water insoluble hydrogel is formed upon interaction of single alginate chains with bi-, tri- or multivalent cations (such as calcium, barium or polylysin). Preferably, purified alginates (e.g. according to DE 198 36 960, the specific disclosure of which is incorporated herein by reference) are used for encapsulation, more preferably potassium or sodium alginates in physiological saline solution. Such alginates typically exhibit an average molar mass of about 20 kDa to about 10,000 kDa, more preferably a molar mass of about 100 kDa to about 1,200 kDa. Alginates used for the formation of the core and/or of the at least one surface coating layer of the (spherical) microcapsule as used according to the present invention, may be provided as a solution, more preferably as an aqueous solution, e.g. the viscosity of a 0.2% (w/v) aqueous alginate solution of the alginate to be used may be in the range of about 2 to about 50 mPa s, more preferably in the range of about 3 to about 10 mPa s. If alginates are used according to the present invention, those, which are rich in α-L-guluronic acid, are preferred. In other words, alginates containing at least 50% α-L-guluronic acid (and less than 50% (3-D-mannuronic acid) are preferred. More preferably, the alginate to be used contains 50% to 70% α-L-guluronic acid and 30 to 50% 13-D-mannuronic acid. Alginates suitable for preparing (spherical) microcapsules as used according to the present invention are obtainable by extraction from certain algae species including, without being limited thereto, brown algae, e.g. Laminarales, Ectocarpales, Fucales, etc., and other species of algae producing alginates. Alginates may be isolated from fresh algae material or dried material according to any method for preparing alginates known to a skilled person.

Cross-linked polymers as defined herein, used for preparation of the (spherical) core of the herein defined (spherical) microcapsule and cross-linked polymers, used for preparation of the at least one surface coating layer of the (spherical) microcapsule may be identical or different with respect to the selected polymer and with respect to the chosen concentrations.

According to a first embodiment the cross-linked polymers used for preparation of the (spherical) core and the at least one surface coating layer may comprise chemically identical polymers in identical or differing concentrations. Preferably, the polymers present in the (spherical) core and the at least one surface coating layer are prepared using a non-cross-linked polymer solution selected from any of the polymers a defined herein. In this polymer solution, the non-cross-linked polymers are typically present in a concentration of about 0.1% (w/v) to about 8% (w/v) of the non-cross-linked polymer, more preferably in a concentration of about 0.1% (w/v) to about 4% (w/v) of the non-cross-linked polymer, even more preferably in a concentration of about 0.5% (w/v) to about 2.5% (w/v) of the non-cross-linked polymer and most preferably in a concentration of about 1% (w/v) to about 2% (w/v) of the non-cross-linked polymer. If alginates as disclosed herein are used as polymers for the preparation of the (spherical) core of the (spherical) microcapsule as used herein and/or used for preparation of the at least one surface coating of the (spherical) microcapsule, the concentration of the polymer solution for preparing the (spherical) core and the concentration of the polymer solution for preparing the at least one surface coating layer of the (spherical) microcapsule, may be selected independently upon each other from a concentration of 0.1 to 4% (w/v) of the non-cross-linked polymer, preferably from a concentration of 0.4 to 2% (w/v) of the non-cross-linked polymer. The alginate concentration for both solutions may be identical. Alternatively, different alginate concentrations may be used for preparing the (spherical) core and the at least one surface coating layer of the (spherical) microcapsules used according to the present invention. Preferably, the non-cross-linked polymers used for preparation of the (spherical) core and/or the at least one surface coating layer comprise chemically identical polymers, more preferably in identical concentrations, e.g. in concentrations as defined herein with polymers as defined herein. In this context the term “% (w/v)” refers to the concentration of non-cross-linked polymers and is typically determined on the basis of a certain amount of a polymer in its dry form versus the total volume of the polymer solution, e.g. after solubilising the non-cross-linked polymer in a suitable solvent (before the cross-linkage). However, the herein concentrations may instead also be meant to correspond to “% v/v” concentrations, if applicable, e.g. if polymers are used, which are present in a fluid aggregate state at standard conditions (room temperature, normal pressure, etc.).

According to a second embodiment the cross-linked polymers used for preparation of the (spherical) core and the at least one surface coating layer may comprise chemically different polymers in identical or differing concentrations. Thereby, concentrations and polymers may be chosen separately as defined herein for the (spherical) core and the at least one surface coating layer independent upon each other. Furthermore, polymers may be chosen from polymers as defined herein, including e.g. natural polymers, synthetic polymers, and combination of polymers, e.g. block polymers. The difference in the nature of the polymers used for the core or the at least one surface coating layer may also be due to different molecular weight of the polymers used and/or due to different cross-linkage of identical polymers, etc.

In case the (spherical) microcapsules comprise more than one surface coating layer, the polymers in each of the at least one surface coating layers may be identical or different, i.e. the cross-linked polymers of each surface coating layer may comprise chemically identical or different polymers in identical or differing concentrations, e.g. the (spherical) microcapsule, as used according to the present invention, may comprise at least one surface coating layer, as defined herein, consisting of any polymer as defined herein, and an additional external surface coating layer consisting of polycations, e.g. polyamino acids as defined herein, e.g. poly-L-lysine, poly-L-glutamic acid, etc. Likewise, the difference in the nature of the polymers used for the differing surface coating layers may be due to a different molecular weight of the polymers used and/or due to different cross-linkage of identical polymers, etc.

The (spherical) core of the (spherical) microcapsule as used herein additionally comprises cells. Such cells are typically selected from stem cells or stromal cells, such as mesenchymal stem cells or mesenchymal stromal cells, or from any other cell (type), that may be used in the context of the present invention, for treatment of AMI or MI diseases or diseases related thereto. Such cells are typically obtainable by stably transfecting a cell with a nucleic acid or rather a vector containing a nucleic acid coding for GLP-1, a fragment or variant thereof or a fusion peptide comprising GLP-1 or a fragment or variant thereof, as defined below.

Cells suitable for the (spherical) core of the (spherical) microcapsule as used herein may be chosen from (non-differentiated) stem cells including totipotent, pluripotent, or multipotent stem cells. Stem cells used in the present context preferably comprise embryonic stem cells or stem cells derived from the ectoderm, the mesoderm or the endoderm, or adult stem cells such as (human) mesenchymal stem cells or mesenchymal stromal cells (MSC, hMSC) (e.g. derived from human bone marrow or from fat tissue), hematopoietic stem cells, epidermal stem cells, neural stem cells and immature fibroblasts, including fibroblasts from the skin (myofibroblasts), etc. These (undifferentiated) stem cells are typically capable of symmetric stem cell division, i.e. cell division leading to identical copies. Stem cells maintain the capacity of transforming into any cell type. Moreover, stem cells are capable of dividing asymmetrically leading to a copy of the stem cell and another cell different from the stem cell copy, e.g., a differentiated cell.

Stem cells as defined herein, particularly mesenchymal stem cells or mesenchymal stromal cells, suitable for the (spherical) core of the (spherical) microcapsule as used herein may additionally produce a set of endogenous trophic factors that support the cytoprotective effect of GLP-1 or of a fragment or variant thereof. Biologically active factors for this paracrine cytoprotective mechanism of the mesenchymal stromal cells may be e.g. the cytokines GRO, IL-6, IL-8, MCP-1 and the growth factors VEGF, GDNF and Neurotrophin-3. According to a particularly preferred embodiment, the cells in the (spherical) core of the (spherical) microcapsules therefore secrete endogenous proteins or peptides as paracrine factors that are released through the capsule in therapeutic levels selected from VEGF, IL6, IL8, GDNF, NT3, and MCP1, etc.

The core of (spherical) microcapsule as used herein, may alternatively contain cells which are chosen from (differentiated) cells, e.g., obtainable from the herein stem cells or stromal cells, e.g., cells of the connective tissue family, e.g., (mature) fibroblasts, cartilage cells (chondrocytes), bone cells (osteoblasts/osteocytes, osteoclasts), fat cells (adipocytes), or smooth muscle cells, or blood cells including lymphoid progenitor cells or cells derived therefrom, e.g., NK cells, T-cells, B-cells or dendritic cells, or common myeloid progenitor cells or cells derived therefrom, e.g., dendritic cells, monocytes, macrophages, osteoclasts, neutrophils, eosinophils, basophils, platelets, megakaryocytes or erythrocytes, or macrophages, neuronal cells including astrocytes, oligodendrocytes, etc., or epithelial cells, or epidermal cells. These differentiated cells are typically capable of symmetric cell division, i.e. cell division leading to identical copies of the differentiated parent cell. Moreover, in some cases these differentiated cells may be capable of dividing asymmetrically leading to an identical copy of the parent cell and another cell different from the parent cell, i.e. a cell being further differentiated than the parent cell. Alternatively, in some cases differentiated cells as defined herein may be capable of differentiating further without the need of cell division, e.g., by adding selective differentiation factors.

Furthermore, cells embedded in the (spherical) core of the (spherical) microcapsule, as used according to the present invention, may be cells taken from the patient to be treated himself (autologous cells) or may be taken from allogenic cells (e.g. taken from an established cell line cultivated in vitro, e.g., HEK293 cells, hTERT-MSC cells, etc.). Due to the surface coating layer embedding the (spherical) core in the (spherical) microcapsule, as used according to the present invention, it allows the use of allogenic cells without evoking any undesired immune response by the patient to be treated.

Cells embedded in the (spherical) core of the (spherical) microcapsule used according to the present invention, may furthermore be a combination of (differentiated and/or non-differentiated) cell types as defined herein. The (spherical) core of the (spherical) microcapsule, as used according to the present invention, may contain, e.g., human mesenchymal stem cells or human mesenchymal stromal cells, wherein a portion of these cells may be differentiated in vitro or in vivo into a cell type, such as defined herein, e.g. adipocytes (suitable for transplantation into fat tissue), etc. Accordingly, various cell types (derived e.g. from a specific stem cell type) may be allocated in the core, e.g. sharing a common lineage.

In summary, cells suitable for preparing the (spherical) core of the (spherical) microcapsule used according to the present invention may be selected from non-differentiated or differentiated cells. According to one embodiment non-differentiated cells as defined herein may be preferred. Such non-differentiated cells may provide advantageous properties, e.g. a prolonged effect of the (spherical) microcapsules used according to the present invention, e.g. the prolonged capability to express and secrete a GLP-1 peptide or a GLP-1 fusion peptide as defined herein, or a fragment or variant thereof, e.g. due to a longer life span of such non-differentiated cells. In an alternative embodiment, differentiated cells as defined herein may be preferred for preparing the (spherical) core of the (spherical) microcapsule used according to the present invention, since they typically do not proliferate any more and, thus, do not lead to any undesired proliferation of cells within the (spherical) core of the (spherical) microcapsule, as used according to the present invention. Specific differentiation of cells may be carried out by a skilled person in vitro according to methods known in the art by adding selected differentiation factors to precursor cells. Preferably, cells are differentiated in such a way that a vast majority of cells (or at least 90%, more preferably at least 95% and most preferably at least 99%) embedded in the (spherical) core of the (spherical) microcapsule used according to the present invention, belongs to the same cell type. In particular, mesenchymal stem cells as defined herein may be differentiated in vitro, e.g., into osteoblasts, chondrocytes, adipocytes such as fat cells, neuron-like cells such as brain cells, etc., and used herein accordingly. As to whether non-differentiated or differentiated cells are used for preparing the (spherical) core of the (spherical) microcapsule, as defined herein, may be dependent on specific requirements of the disease to be treated, e.g. the site of affliction, the administration mode, the tissue chosen for implant, etc. A selection of appropriate cells may be carried out by a skilled person evaluating these criteria.

Furthermore, cells suitable for preparing the (spherical) core of the (spherical) microcapsule as defined herein may be immortalised or non-immortalised cells, preferably immortalised cells. If immortalised cells are used, these cells preferably retain their capability of symmetric and/or asymmetric cell division as discussed herein. According to the present invention cells are defined as immortal when they exceed the double life span of normal cells (i.e. of non-immortalised cells). The maximum life span of normal diploid cells in vitro varies dependent on the cell type (e.g. foetal versus adult cell) and culture conditions. Thus, the maximum life span of cultured normal cells in vitro is approximately 60-80 population doublings. For example, keratinocytes may divide around 80 times, fibroblasts more than 50 times, and lymphocytes about 20 times. Normal bone marrow stromal cells may exhibit a maximum life span of 30-40 population doublings. Preferably, a cell line used for preparation of the (spherical) core of an (spherical) microcapsule, as used according to the present invention, may continuously grow past 350 population doublings and may still maintain a normal growth rate characteristic of young cells.

Methods for immortalising cells for preparing the (spherical) core of the inventive (spherical) microcapsule as defined herein are widely known in the art and may be applied here accordingly (see e.g. WO 03/010305 or WO 98/66827, which are incorporated herein by reference). An exemplary method (according to WO 03/010305) comprises e.g. following steps:
a) culturing cells, e.g., stem cells, in particular stem cells derived from human bone marrow (e.g. (human) mesenchymal stem cells (MSC, hMSC)), in accordance with standard conventional cell culturing methods known to the skilled person;
b) transducing said cell cultures with a retroviral vector, comprising at least a fragment of the human telomerase reverse transcriptase (hTERT) gene or a variant thereof, by
   b1) culturing a packaging cell line (e.g. PA317 cells, PG13 cells, Phenix, etc.), wherein the packaging cell line are cells in which the retroviral vector is produced,
   b2) constructing a retroviral vector (e.g. derived from Moloney murine leukaemia virus, etc.), wherein the retroviral vector comprises at least a fragment of the catalytic subunit of the human telomeric repeat (hTRT) gene or a variant thereof, more preferably a hTERT cDNA fragment, e.g. a 3452 base pair EcoRI fragment from pGRN145 (Geron Corporation),
   b3) transfecting said packaging cell line, with said retroviral vector,
   b4) transducing said packaging cell line with said transfected cells, preferably by centrifuging the cells with the retroviral vector,
   b5) transducing cultured cells according to step a) herein with the packaging cells of step b4), said cells comprising said retroviral vector.
c) obtaining an immortal cell line, wherein said immortalised cell line has substantially identical characteristics and properties compared to the cells of step a). As a result the inserted polynucleotide sequence derived from the human telomeric subunit (hTRT) gene may be transcribed and translated to produce a functional telomerase. One of skill will recognize that due to codon degeneracy a number of polynucleotide sequences will encode the same telomerase. In addition, telomerase variants are included, which have sequences substantially identical to a wildtype telomerase sequence and retain the function of the wildtype telomerase polypeptide (e.g. resulting from conservative substitutions of amino acids in the wildtype telomerase polypeptide).

Cells embedded in the (spherical) core of the (spherical) microcapsule encoding and secreting the GLP-1 peptides and GLP-1 fusion peptides as defined herein may be further modified or engineered to additionally secrete a factor selected from the group consisting of anti-apoptotic factors, growth factors, VEGF, erythropoietin (EPO), anti-platelet factors, anti-coagulant factors, anti-thrombotic drugs, anti-angiogenic factors, or any further factor exhibiting cardioprotective function, etc.

According to one specific embodiment, the cells embedded in the core of the (spherical) microcapsule encoding and secreting the GLP-1 peptides and GLP-1 fusion peptides as defined herein may be engineered to additionally secrete erythropoietin (EPO). Erythropoietin (also known as EPO, epoetin or procrit) is an acidic glycoprotein hormone of approximately 34,000 dalton molecular weight occurring in multiple forms, including alpha, beta, omega and asialo. Erythropoietin stimulates red blood cell production. It is produced in the kidney and stimulates the division and differentiation of committed erythroid precursors in the bone marrow and elsewhere. Generally, erythropoietin is present in very low concentrations in plasma when the body is in a healthy state, in which tissues receive sufficient oxygenation from the existing number of erythrocytes. This normal low concentration is enough to stimulate replacement of red blood cells that are lost normally through aging. The amount of erythropoietin in the circulation is increased under conditions such as hypoxia, when oxygen transport by blood cells in the circulation is reduced. Hypoxia may be caused by loss of large amounts of blood through haemorrhage, destruction of red blood cells by over-exposure to radiation, reduction in oxygen intake due to high altitudes or prolonged unconsciousness, or various forms of anaemia or ischemia. In response to tissues undergoing hypoxic stress, erythropoietin will increase red blood cell production by stimulating the conversion of primitive precursor cells in the bone marrow into proerythroblasts which subsequently mature, synthesize haemoglobin and are released into the circulation as red blood cells. When the number of red blood cells in circulation is greater than needed for normal tissue oxygen requirements, erythropoietin in circulation is decreased. Preferably, erythropoietin is used as an additional factor contained in the cells to induce production of red blood cells to combat anaemia. (See, e.g., Bottomley et al. (2002) Lancet Oncol. 3:145). Erythropoietin has also been suggested to be useful in controlling bleeding in patients with abnormal haemostasis. (See e.g., U.S. Pat. No. 6,274,158). Recombinant human erythropoietin (rHuEpo or epoetin [alpha]) is commercially available as EPOGEN® (epoetin alfa, recombinant human erythropoietin) (Amgen Inc., Thousand Oaks, Calif.) and as PROCRIT® (epoetin alfa, recombinant human erythropoietin) (Ortho Biotech Inc., Raritan, N.J.). EPO may increase the hematocrit values in patients suffering from AMI or MI. The normal ranges for hematocrit values of erythropoietin are 37-48 percent for women and 42-52 percent for men. (See Case Records of the Massachusetts General Hospital: normal reference laboratory values. (1992) N. Eng. J. Med. 327:718). Of course, the safety and efficacy of use of erythropoietin to increase hematocrit levels in patients with cardiovascular disease, especially those suffering from renal failure, must be further evaluated. Preferably, erythropoietin is typically provided at a concentration or for a duration that will not induce red blood cell formation or alternatively, increase the hematocrit in a subject, e.g., between about 1 pM and less than 1000 μM, including less than 900 μM, less than 700 μM, less than 500 μM, less than 300 μM, less than 100 μM, or less than 50 μM. In other embodiments, erythropoietin is administered as a function of the subject's body weight. Erythropoietin may typically be provided at a concentration of between about 1 U/kg to 10,000 U/kg of a subject's body weight, including less than 7,500 U/kg, 5,000 U/kg, 2500 U/kg, 1000 U/kg, 750 U/kg, 500 U/kg, 250 Ug/kg, 100 Ug/kg, 50 U/kg, 25 U/kg, 10 U/kg, 5 U/kg, or 1 U/kg. In this context, erythropoietin serum concentration is normally within the range of 5-50 mU/ml. For patients suffering from MI or AMI or other conditions associated thereto, erythropoietin is preferably provided either at a concentration of 50-100 U/kg depending on symptom, body weight, sex, animal species and the like. It is generally assumed that treatment options holding the blood concentration at about 1-100 mU/ml will be preferred. Also preferably, erythropoietin is typically provided at a concentration that does not increase the hematocrit in a survivor, wherein the erythropoietin is administered in a single dose within 1, 2 or 3 hours of the myocardial infarction, for an extended period of time.

According to one further specific embodiment, the cells embedded in the core of the (spherical) microcapsule encoding and secreting the GLP-1 peptides and GLP-1 fusion peptides as defined herein may be engineered to additionally secrete VEGF.

According to another specific embodiment, the cells embedded in the core of the (spherical) microcapsule encoding and secreting the GLP-1 peptides and GLP-1 fusion peptides as defined herein may be engineered to additionally secrete antiapoptotic factors. Such factors may include, without being limited thereto, APC (apoptosis repressor with caspase recruitment domain), Bcl-2, Bcl-xL, Che-1/AATF, clusterin, insulin, Mcl-1, NF-kB-dependent anti-apoptotic factors, serotonin, survivin, etc. Furthermore, any factor, which acts as an inhibitory factor to an apoptotic factor known in the art, and which may thus be regarded as antiapoptotic factors, is encompassed herewith. Such factors are preferably encoded by a nucleic acid and secreted by the cells encoding and secreting the GLP-1 peptides and GLP-1 fusion peptides as defined herein. In this context, such antiapoptotic factors may be directed against at least one of the following apoptotic factors or apoptosis related proteins including AIF, Apaf e.g. Apaf-1, Apaf-2, Apaf-3, oder APO-2 (L), APO-3 (L), Apopain, Bad, Bak, Bax, Bcl-2, Bcl-x_{L}, Bcl-x_{S}, bik, CAD, Calpain, Caspase e.g. Caspase-1, Caspase-2, Caspase-3, Caspase-4, Caspase-5, Caspase-6, Caspase-7, Caspase-8, Caspase-9, Caspase-10, Caspase-11, ced-3, ced-9, c-Jun, c-Myc, crm A, cytochrom C, CdR1, DcR1, DD, DED, DISC, DNA-PK_{CS}, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, transglutaminase, etc.

A GLP-1 peptide encoded and secreted by a cell contained in the (spherical) core of the (spherical) microcapsule, as defined herein, may be selected from any known GLP-1 peptide sequence. In this context, the neuroprotective factor GLP-1 is located on the well studied glucagon gene, which encodes preproglucagon (see e.g. White, J. W. et al., 1986 Nucleic Acid Res. 14(12) 4719-4730). The preproglucagon molecule as a high molecular weight precursor molecule is synthesized in pancreatic alpha cells and in the jejunum and colon L cells. Preproglucagon is a 180 amino acid long prohormone and its sequence contains, in addition to glucagon, two sequences of related structure: glucagon-like peptide-1 (GLP-1) and glucagon-like peptide-2 (GLP-2). In the preproglucagon molecule, between GLP-1 and GLP-2 is a 17 amino acid peptide sequence (or rather a 15 amino acid sequence plus the C-terminal RR cleavage site), intervening peptide 2 (IP2). The IP2 sequence (located between GLP-1 and GLP-2 in the precursor molecule) is normally cleaved proteolytically after aa 37 of GLP-1 in vivo. The preproglucagon module is therefore cleaved into various peptides, depending on the cell, and the environment, including GLP-1 (1-37), a 37 amino acid peptide in its unprocessed form. Generally, this processing occurs in the pancreas and the intestine. The GLP-1 (1-37) sequence can be further proteolytically processed into active GLP-1 (7-37), the 31 amino acid processed form, or its further degeneration product GLP-1 (7-36) amide. Accordingly, the designation GLP-1(7-37) means that the fragment in question comprises the amino acid residues (starting) from (and including) number 7 to (and including) number 37 when counted from the N-terminal end of the parent peptide, GLP-1.

The amino acid sequence of GLP-1(7-36), GLP-1(7-36)amide and of GLP-1(7-37) is given in formula I (SEQ ID NO: 25):

His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-X (I)

which shows GLP-1(7-36)amide when X is NH₂ or GLP-1(7-36), when X is absent, and GLP-1(7-37) when X is Gly-OH.

According to one embodiment of the present invention, the GLP-1 peptide may therefore be selected from any known GLP-1 peptide sequence, e.g. as defined herein. In this context, the GLP-1 peptide may be secreted by cells embedded in the (spherical) core of the (spherical) microcapsule which thus may be transfected preferably prior to preparing the (spherical) core with nucleic acid sequences encoding a GLP-1 peptide as defined herein such that these cells express and secrete the GLP-1 peptide. Preferably a GLP-1 peptide as used herein, which may be encoded and secreted by a cell embedded in the (spherical) microcapsule, may be selected from a group consisting of a peptide comprising aa 7-35 of (wt) GLP-1 or a peptide showing an identity of at least 80%, 90%, 95% or even 99% with this peptide. In general, the GLP-1 peptide may be selected from group consisting of (i) a peptide comprising aa 1-37 of (wt) GLP-1, (ii) a peptide comprising aa 7-35, 36 or 37 of (wt) GLP-1, (iii) GLP-1(7-36)amide and (iv) a peptide showing an identity of at least 80%, 90%, 95% or even 99% with any of these peptides, including modified peptides. In this context, a “modified GLP-1 peptide” is intended to mean any GLP-1 variant or a GLP-1 fragment, including combinations, e.g. a fragment of a variant, which retain the biological function of (wt) GLP-1. Variants and fragments are categorized as modifications of the unmodified GLP-1 sequence, e.g. GLP-1(7-35, 36 or 37). Within the meaning of the present invention any variant or fragment has to be functional, e.g. has to exert the same or a similar biological activity as the unmodified (GLP-1) peptide. The term “activity” refers to the biological activity (e.g. one or more of the biological activities comprising receptor binding, activation of the receptor, exhibition of beneficial effects known for GLP-1, e.g. its activity to powerfully reduce the damages caused by ischemia or oxygen shortage and potential death of heart tissue as mentioned herein in connection with the effects of GLP-1 as described in the prior art, which may be compared under the same conditions for the naturally occurring GLP-1 peptide as defined herein and any fragment or variant thereof.

Preferably, a variant or fragment of a GLP-1 peptide as defined herein exerts at least 25% activity of a GLP-1(7-35, 36 or 37), more preferably at least 50% (biological) activity, even more preferably 60, 70, 80 or 90% (biological) activity and most preferably at least 95 or 99% (biological) activity of a GLP-1(7-35, 36 or 37) as defined herein. The biological activity may be determined by a standard assay, e.g. which preferably allows determining the activity as an incretin hormone lowering the blood glucose level, e.g. using an animal model for diabetes type 2, etc.

According to a particularly preferred embodiment, the GLP-1 peptide or a GLP-fusion peptide as defined herein, which may be as encoded by cells embedded in the (spherical) core of the (spherical) microcapsule, does not include at its N-terminus the naturally occurring amino acids 1 to 6 of a (native) GLP-1 (1-37) sequence as defined herein. Even more preferably, the GLP-1 peptide as defined herein or a GLP-fusion peptide as defined below does not include at its N-terminus the naturally occurring amino acids 1, 2, 3, 4, 5 and/or 6 of a native GLP-1 (1-37) sequence as defined herein. This proviso preferably refers to GLP-1 peptides as defined herein, e.g. selected from the group consisting of a peptide comprising aa 7-35, 36 or 37 of GLP-1, GLP-1(7-36)amide and a peptide showing an identity of at least 80%, 90%, 95% or even 99% with any of these peptides, including modified peptides, and to GLP-1 fusion peptides containing such GLP-1 peptides. However, this proviso does not exclude, that such a GLP-1 peptide as defined herein or a GLP-1 fusion peptide as defined herein, comprises an N-terminal (or C-terminal) sequence modification or additional amino acids or peptides fused thereto, e.g. signal peptide sequences and/or leader peptide sequences, etc., however being distinct from the sequence of amino acids 1 to 6 of wt GLP-1. In another preferred embodiment, any amino acid attached to the N-terminus of GLP-1 (7-35, 36 or 37) of homologs thereof does not correspond to the naturally occurring amino acid at position 6 of GLP-1(7-35, 36 or 37). According to a further preferred embodiment, any amino acid (directly) attached to the N-terminus of GLP-1 (7-35, 36 or 37) of homologs thereof does not correspond to the naturally occurring amino acid 6, to the naturally occurring amino acids 5 and 6, to the naturally occurring amino acids 4, 5 and 6, to the naturally occurring amino acids 3, 4, 5, and 6, to the naturally occurring amino acids 2, 3, 4, 5, and 6 or to the naturally occurring amino acids 1, 2, 3, 4, 5, and 6 of native GLP-1, preferably in their native order in GLP-1. According to a particularly preferred embodiment, any amino acid attached to the N-terminus of GLP-1 (7-35, 36 or 37) of homologs thereof does not correspond to the sequence of preproglucagon.

Native GLP-1, particularly GLP-1 (7-36), suffers from a short half life in vivo and therefore is of limited use in therapeutic treatments in general, where a frequent administration is strictly to be avoided or where a long-term administration is envisaged. GLP-1 is rapidly degraded in plasma within minutes by DPP-IV (dipeptidyl peptidase IV) between residues 8 and 9, resulting in an inactive NH₂-terminally truncated metabolite GLP-1 (9-36). Additionally, native GLP-1 typically undergoes renal excretion. These factors raise the issue, as to which peptide, GLP-1 (7-36) or the NH₂-terminally truncated metabolite GLP-1 (9-36), is the active moiety in vivo and as to whether physiological effects are exerted in therapeutic applications by the native GLP-1 or its fragments. As a consequence and due to its rapid degradation in vivo, native GLP-1 or its fragments may be used as a suitable tool for a short-term metabolic control, such as intensive care units potentially useful in patients with acute AMI or MI diseases or diseases related thereto.

To avoid such fast degradation, various attempts have been made to synthesize stabilized (against degradation by DPP-IV) analogues of naturally occurring GLP-1 (e.g. GLP-1(7-37)). In particular, the 8^{th} residue, which in vivo is Ala, was replaced by another residue, for instance, Gly, Ser or Thr (Burcelin, R. et al. (1999) Metabolism 48, 252-258). The Gly8 (or G8) analogue has been extensively tested, both as synthesized molecule, and produced by cell lines genetically engineered to secrete the mutant polypeptide (Burcelin, R., et al. (1999), Annals of the New York Academy of Sciences 875: 277-285). Various other modifications have been introduced into e.g. GLP-1(7-37) to enhance its in vivo stability without compromising its biological activity.

Such an approach circumvents the problem of short half life by stabilization of GLP-1 against degradation by DPP-IV, e.g. by additionally administering a DPP-IV inhibitor with the GLP-1 peptide. Additionally administering a DPP-IV inhibitor with the GLP-1 peptide is complicated and typically does not lead to the desired long-term treatment as the DPP-IV inhibitor may only be used efficiently in in vitro systems.

Therefore, according to an alternative embodiment, a GLP-1 peptide encoded and secreted by cells embedded in the core of the (spherical) microcapsule may be selected from a GLP-1 fusion peptide or a variant or fragment thereof. The GLP-1 fusion peptide as used herein may be encoded and secreted by cells embedded in the (spherical) core of the (spherical) microcapsule as defined herein. In this context, cells embedded in the (spherical) core of the (spherical) microcapsule, as defined herein, are typically transfected prior to preparing the core with nucleic acid sequences encoding the GLP-1 fusion peptide such that these cells encode, express and secrete the GLP-1 fusion peptide.

The GLP-1 fusion peptides as defined herein preferably have at least two components, e.g. components (I) and (II), components (I) and (III) or components (I), (II) and (III), exhibit GLP-1's biological activity as defined herein and, simultaneously, confer stability to component (I) of GLP-1 fusion peptides typically by (such) a C-terminal elongation. Component (I) of GLP-1 fusion peptides as defined herein typically contains a sequence of a GLP-1 peptide as defined herein, preferably a sequence having at least 80%, more preferably at least 85% and even more preferably at least 90% sequence identity with SEQ ID NO: 1. SEQ ID NO:1 represents the native amino acid sequence of GLP-1(7-37) (length of 31 amino acids), which is strictly conserved among mammalians. According to a particularly preferred embodiment, component (I) of GLP-1 fusion peptides as defined herein contains a sequence being identical to SEQ ID NO: 1 or a sequence, which lacks amino acids 36 and/or 37 of SEQ ID NO: 1.

Component (II) of the GLP-1 fusion peptide, which may be encoded and secreted by cells embedded in the (spherical) core of the (spherical) microcapsule as defined herein, (or more generally any GLP-1 peptide including fragments or variants of fusion peptides) typically contains a peptide sequence having at least nine amino acids. The GLP-1 fusion peptide may typically have in its component (II) a sequence length of 9 to 30, preferably 9 to 20, and most preferably 9 to 15 amino acids. Generally spoken, shorter sequences in component (II) may be preferred due to their superior binding activity to the GLP receptor over longer sequences. The sequence of component (II), even though it is not a prerequisite, may preferably be neutral or may have a negative charge at pH7. Component (II) of the GLP-1 fusion peptide furthermore may contain at least one proline residue in its sequence. Proline residues are common amino acids within a n-turn forming tetrameric amino acid sequence. Thus, component (II) of the GLP-1 fusion peptide may form a (3-turn like structure. A β-turn structure is a typical secondary structure element of proteins or peptides. It is typically formed by a stretch of four amino acids, which reverts the direction of the peptide's or protein's backbone chain direction. If present in the GLP-1 fusion peptide, the proline residue is commonly located at position 2 or 3, preferably at position 2, of a tetrameric β-turn sequence motif occurring in component (II) of the GLP-1 fusion peptide.

Component (II) of the GLP-1 fusion peptide, which may be encoded and secreted by cells embedded in the (spherical) core of the (spherical) microcapsule as defined herein, (or more generally any GLP-1 peptide including fragments or variants of fusion peptides) may contain a sequence motif selected from the group consisting of VAIA, IAEE, PEEV, AEEV, EELG, AAAA, AAVA, AALG, DFPE, AADX, AXDX, and XADX, wherein X represents any amino acid (naturally occurring or a modified non-natural amino acid). These tetrameric motifs may be located anywhere in the sequence of component (II). In a particularly preferred embodiment, the inventive fusion peptide component (II) is a peptide sequence being linked to the C-terminus of component (I) by its N-terminal sequence motif selected from the group consisting of AA, XA, AX, RR, RX, and XR, wherein X represents any amino acid (naturally occurring or a modified non-natural amino acid).

Particularly preferred as component (II) of a GLP-1 fusion peptide, which may be encoded and secreted by cells embedded in the (spherical) core of the (spherical) microcapsule as defined herein, is a peptide sequence containing a sequence according to SEQ ID NO: 48: X₁X₁DFPX₂X₂X₃X₄, corresponding to a partial sequence of human or murine IP-2, wherein each X₁ is typically selected independently upon each other from any naturally occurring amino acid, preferably arginine (R) or alanine (A), more preferably alanine (A), or may be absent; wherein each X₂ is typically selected independently upon each other from aspartic acid (D) or glutamic acid (E), and wherein each X₃ and X₄ is typically selected independently upon each other from any naturally occurring amino acid, preferably alanine (A), glycine (G), isoleucine (I), leucine (L), threonine (T), or valine (V). X₄ also may be absent.

Even more preferred as component (II) of a GLP-1 fusion peptide, which may be encoded and secreted by cells embedded in the (spherical) core of the (spherical) microcapsule as defined herein, is a peptide sequence containing a sequence according to SEQ ID NO: 22 (RRDFPEEVAI), SEQ ID NO: 27 (DFPEEVAI), SEQ ID NO: 28 (RDFPEEVA), or SEQ ID NO: 29 (RRDFPEEV), SEQ ID NO: 30 (AADFPEEVAI), SEQ ID NO: 31 (ADFPEEVA), or SEQ ID NO: 32 (AADFPEEV), (all peptide sequences given in the one-letter-code) or a sequence having at least 80% sequence identity with SEQ ID NO: 22, 27, 28, 29, 30, 31 or 32. SEQ ID NO: 22 is a partial sequence of the full-length IP-2 (intervening peptide 2) sequence, which contains the 10 N-terminal amino acids of the 15 amino acid long full-length IP-2 sequence. IP-2 is a preferred example of a component (II) as used herein. Accordingly, other stronger preferred sequences being contained in component (II) of the herein defined GLP-1 fusion peptide are longer partial amino acid sequences of IP-2, such as the 14 N-terminal amino acid sequence occurring in humans (SEQ ID NO: 23 (RRDFPEEVAIVEEL)) or its murine counterpart (SEQ ID NO: 24 (RRDFPEEVAIAEEL)), or sequences (SEQ ID NO: 33 (AADFPEEVAIVEEL)) or (SEQ ID NO: 34 (AADFPEEVAIAEEL)), or a sequence having at least 80% sequence identity with SEQ ID NOs: 23, 24, 33 or 34. Most preferred as elements being contained in component (II) of the GLP-1 fusion peptide are full-length IP-2 sequences having all 15 amino acids of the naturally occurring IP-2 sequence (SEQ ID NO: 2 (RRDFPEEVAIVEELG), human, or SEQ ID NO: 3 (RRDFPEEVAIAEELG), murine, or SEQ ID NO: 35 (AADFPEEVAIVEELG), or SEQ ID NO: 36 (AADFPEEVAIAEELG)) or a sequence having at least 80% sequence identity with SEQ ID NOs: 2, 3, 35 or 36. Within the scope of the present invention are also all mammalian isoforms of IP2 (natural variants of IP2 among mammalians). More than one copy of a sequence being included into component (II) may be provided, e.g. 2, 3 or even more copies of IP2 or a fragment or variant of IP2.

Accordingly, a GLP-1 fusion peptide, encoded and secreted by cells embedded in the (spherical) core of the (spherical) microcapsule, as defined herein, preferably contains, comprises or consists of sequences according to SEQ ID NO: 8 (HAEGTFTSDVSSYLEGQAAKEFIAWLVKGRGRRDFPEEVAIAEELG), i.e. GLP-1(7-37) linked without any linker sequence via its C-terminus to murine IP2 or according to SEQ ID NO: (HAEGTFTSDVSSYLEGQAAKEFIAWLVKGRGRRDFPEEVAIVEELG), i.e. GLP-1(7-37) linked without any linker sequence via its C-terminus to human IP2, or sequences according to SEQ ID NO: 37 (HAEGTFTSDVSSYLEGQAAKEFIAWLVKGRGAADFPEEVAIAEELG), i.e. GLP-1(7-37) linked without any linker sequence via its C-terminus to IP2 or according to SEQ ID NO: 38 (HAEGTFTSDVSSYLEGQAAKEFIAWLVKGRGAADFPEEVAIVEELG), i.e. GLP-1(7-37) linked without any linker sequence via its C-terminus to IP2, or a sequence SEQ ID NO: 39 (HAEGTFTSDVSSYLEGQAAKEFIAWLVKGRGRRDFAEEVAIAEELG), SEQ ID NO: 40 (HAEGTFTSDVSSYLEGQAAKEFIAWLVKGRGRRDAAAAVAIAEELG), SEQ ID NO: 41 (HAEGTFTSDVSSYLEGQAAKEFIAWLVKGRGAADAAAAVAIAAALG), SEQ ID NO.: 42 (HAEGTFTSDVSSYLEGQAAKEFIAWLVKGRGRRDFP), SEQ ID NO: 43 (HAEGTFTSDVSSYLEGQAAKEFIAWLVKGRGRRDFPEEVA), SEQ ID NO: 44 (HAEGTFTSDVSSYLEGQAAKEFIAWLVKGRGRRDFPEEVAIAEELGRRHAC), SEQ ID NO: 45 (HAEGTFTSDVSSYLEGQAAKEFIAWLVKGRGRRDFAEEVAIVEELG), SEQ ID NO: 46 (HAEGTFTSDVSSYLEGQAAKEFIAWLVKGRGRRDAAAAVAIVEELG), SEQ ID NO: 47 (HAEGTFTSDVSSYLEGQAAKEFIAWLVKGRGAADAAAAVAIVAALG), or SEQ ID NO: 48 (HAEGTFTSDVSSYLEGQAAKEFIAWLVKGRGRRDFPEEVAIVEELGRRHAC), i.e. GLP-1(7-37) linked without any linker sequence via its C-terminus to specific analogs or variants of the IP2 sequence. Variants or fragments thereof having a sequence identity of at least 80% with SEQ ID NOs: 8, 12, and 37 to 48, or fragments or variants thereof may be used herein as well. Preferred GLP1-fusion peptides in this context may further comprise sequences according to SEQ ID NOs: 13, 14, 19 and 20.

Without being bound to any theory, it is concluded by the inventors of the present invention that the instability of GLP-1(7-35, 36 or 37), e.g. if secreted in vivo into the patients surrounding tissue by cells embedded in the (spherical) core of the implanted (spherical) microcapsule used according to the present invention, is due to its unprotected 3-dimensional structure. Proteases may cleave the GLP-1(7-35, 36 or 37) peptide and abolish its physiological activity rapidly in vivo. By linking a peptide sequence to the C-terminus of GLP-1(7-35, 36 or 37) its structure gains stability towards enzymatic degradation. Such gain in stability may be enhanced, if the additional C-terminal peptide sequence (being contained in component (II) of the fusion peptide according to the invention) folds back, e.g. due to the presence of a β-turn structural element formed by its primary structure and providing rigidity to component (II). The GLP-1 fusion peptide as defined herein, by virtue of its C-terminal peptide extension preferably containing a β-turn structural element, is found to have improved resistance to DPP-IV inactivation. The C-terminal peptide is either not cleaved from the GLP-1(7-35, 36 or 37) sequence prior to acting on its receptor in target cells or it may be cleaved enzymatically to form GLP-1(7-35, 36 or 37) in vivo. Irrespective of the exact form of the GLP-1 peptide bound at the site of the GLP-1 receptor, a GLP-1 peptide as defined herein exerts its function as an active neuroprotective compound. GLP-1 peptide sequences, which are considered to be suitable for component (II) of a GLP-1 fusion peptide as defined herein due to a primary structure forming a β-turn element, may readily be identified by adequate, e.g., spectroscopic methods, e.g. circular dichroism, or other methods known to the skilled person.

Component (II) and component (I) of a GLP-1 fusion peptide, which may be encoded and secreted by cells embedded in the (spherical) core of the (spherical) microcapsule as defined herein, may be directly linked or linked via a linker sequence. Preferably, both components are directly linked with each other. In case they are linked via a linker (or spacer), the linker is preferably a peptide linker. The peptide linker typically has a length of 1 to 10 amino acids, preferably 1 to 5, even more preferably 1 to 3 amino acids, in some cases the linker sequence may be even longer comprising 11 to 50 amino acids. The peptide linker may be composed of various (naturally occurring) amino acid sequences. Preferably, the peptide linker will introduce some structural flexibility between components to be linked. Structural flexibility is achieved e.g. by having a peptide linker containing various glycine or proline residues, preferably at least 30%, more preferably at least 40% and even more preferably at least 60% proline and glycine residues within the linker sequence. Irrespective of the specific sequence the peptide linker may preferably be immunologically inactive.

GLP-1 fusion peptides, which may be encoded and secreted by cells embedded in the (spherical) core of the (spherical) microcapsule as defined herein, may additionally contain a component (III). Generally, component (III) comprises at least four amino acid residues, preferably at least 10 additional amino acid residues, more preferably at least 20, or most preferably at least 30. In functional terms, component (III) is intended to further enhance the stability of a GLP-1 peptide as defined herein. Component (III) is expected not to interfere with the biological function of the GLP-1 fusion peptide, which is approximately comparable to the biological activity of GLP-1(7-37). Generally spoken, any C-terminal elongation of component (I) as defined herein, whether it is component (II), component (III) or a combination of components (II) and (III) as defined herein, enhances stability of component (I), i.e. a GLP-1 peptide as defined herein, e.g. GLP-1(7-35, 36 or 37), or its fragments or variants as defined herein.

Preferably, component (III) of the GLP-1 fusion peptide as defined herein, comprises at least 4, preferably at least 10, more preferably at least 20 additional amino acid residues of the N-terminal sequence of an isoform of GLP-2 of any mammalian organism (other naturally occurring variant of GLP-2 among mammalian), e.g. murine or human isoforms as shown in SEQ ID NOs: 4 and 5. GLP-2 occurs in pro-glucagon and is also involved in carbohydrate metabolism. In the context of the present invention, the term “GLP-2 peptide” preferably means GLP-2 (1-33, 34, or 35), whereas “modified GLP-2 peptide” is intended to mean any GLP-2 fragment or variant, or a fragment or variant of GLP-2(1-33, 34 or 35). Variants or fragments are categorized as modifications of the unmodified sequence, e.g. GLP-2(1-33, 34 or 35). As with the biologically active sequence included in component (I) (GLP-1 peptide), component (III) may also comprise variants or fragments of naturally occurring forms of GLP-2. Alternatively, component (III) may also comprise at least 4, preferably at least 10, more preferably at least 20 additional amino acid residues of the (N-terminal) sequence of GLP-1(7-37), correspondingly including all mammalian isoforms or—as disclosed herein—all functional fragments or variants thereof. Generally speaking, component (III) may contain any form of a GLP-1 peptide or a modified GLP-1 peptide, which is disclosed herein as suitable for component (I) of the GLP-1 fusion peptide. In a further alternative, component (III) may also contain chimeric forms of GLP-1(7-37) and GLP-2. A chimeric form may be produced by coupling GLP-1(7-37) and GLP-2 (or fragments or variants) with each other and by subsequently introducing this chimeric form as component (III) into the GLP-1 fusion peptide. Preferably, the chimeric form is composed of a partial sequence of GLP-1(7-37) and a partial sequence of GLP-2 linked together. E.g. the chimeric form may include the N-terminal 5 to 30 amino acids of GLP-1 and the C-terminal 5 to 30 amino acids of GLP-2 or vice versa, e.g. amino acids 7 or 8 to 22, 23, 24, 25, 26, 27, or 28 of GLP-1(7-37) and amino acid sequence from position 15, 16, 17, 18, 19, 20, 21, 22, 23 or 24 to e.g. the C-terminus of GLP-2. If modifications of naturally occurring forms of GLP-2 or GLP-1(7-37), respectively, are contained as component (III), component (III) preferably contains the sequence of SEQ ID NOs: 1, 4 or 5, respectively, or a sequence having at least 80% sequence identity with any of SEQ ID NOs: 1, 4 or 5.

In another embodiment, component (III) of the GLP-1 fusion peptide, which may be encoded and secreted by cells embedded in the (spherical) core of the (spherical) microcapsule as defined herein, may contain a plurality of sequences as described herein for components (I), (II) or (III). E.g. component (III) may contain at least two, preferably 2, 3, or 4 copies of GLP-1(7-37) and/or GLP-2 or at least two copies of sequences having at least 80% sequence identity with SEQ ID NOs: 1, 4 or 5. Also, component (III) may contain more than one copy of a chimeric version of GLP-1(7-37) or GLP-2, as disclosed herein, e.g. eventually forming a combination of chimeric version(s) together with GLP-1(7-37) and/or GLP-2 or its modifications with at least 80% sequence identity. A GLP-1 fusion peptide, which may be encoded and secreted by cells embedded in the (spherical) core of the (spherical) microcapsule as defined herein may also comprise two or more, preferably two, components (III), which may e.g. be (1) linked by its N-terminus to the C-terminus of component (I) or (II) and (2) linked by its C-terminus to the N-terminus of component (I) via a linker or directly. If two components (III) are provided, these may be identical or different.

According to a preferred embodiment, a GLP-1 fusion peptide, encoded and secreted by cells embedded in the (spherical) core of the (spherical) microcapsule as defined herein, may comprise the herein defined components (I), (II) and (III). Specific embodiments containing all of these components are preferably selected from a group consisting of: SEQ ID NO: 6 (N-GLP-1(7-37)-IP2(murine)-RR-GLP-1(7-37)-C, also designated murine CM1 herein), SEQ ID NO: 7 (N-GLP-1(7-37)-IP2(murine)-RR-GLP2-C, also designated murine CM2 herein), SEQ ID NO: 10 (N-GLP-1(7-37)-IP2(human)-RR-GLP-1(7-37)-C, also designated human CM1), and SEQ ID NO: 11 (N-GLP-1(7-37)-IP2(human)-RR-GLP-2-C), also designated human CM2 herein) or a sequence having at least 80% sequence identity with SEQ ID NOs: 6, 7, 10, or 11 or a fragment or variant thereof. In the (directly) afore-mentioned sequences the terms “N” and “C” indicate N- and the C-terminus of these fusion peptides. All sequences according to SEQ ID NOs: 6, 7, 10 and 11 contain an RR-Linker (two arginine residues) at the C-terminus of IP2 (component (II)), which may alternatively also be discarded. Component (I) in each of the embodiments according to SEQ ID NOs: 6, 7, 10 or 11 is GLP-1 (7-37), whereas component (III) (in each of these embodiments linked to the C-terminus of component (II)) is either GLP-1(7-37) or GLP-2. Preferred GLP1-fusion peptides in this context may further comprise sequences according to SEQ ID NOs: 15, 16, 17, 18 and 26.

In another preferred embodiment of the present invention, a GLP-1 fusion peptide, which may be encoded and secreted by cells embedded in the (spherical) core of the (spherical) microcapsule, as defined herein, contains in addition to component (I) a component (III) (without any component (II) as defined herein) which is either linked to the C-terminus of component (I) and/or to the N-terminus of component (I). Preferably, component (III) is located at the C-terminus of component (I). Irrespective of whether component (III) is linked to the N-terminus of component (I) (by its C-terminus) or to the C-terminus of component (I) (by its N-terminus), the coupling may be direct or indirect via a linker sequence. With regard to the linker sequence it is referred to the herein disclosure of GLP-1 fusion peptides for a linker connecting component (I) and component (II) of the GLP-1 fusion peptide.

In an alternative preferred embodiment of the present invention, a GLP-1 fusion peptide, which may be encoded and secreted by cells embedded in the (spherical) core of the (spherical) microcapsule, as defined herein, contains in addition to components (I) and (II) a component (III) which is either linked to the C-terminus of component (II) and/or to the N-terminus of component (I). Preferably, component (III) is located at the C-terminus of component (II). Irrespective of whether component (III) is linked to the N-terminus of component (I) (by its C-terminus) or to the C-terminus of component (II) (by its N-terminus), the coupling may be direct or indirect via a linker sequence. With regard to the linker sequence it is again referred to the herein depicted disclosure of GLP-1 fusion peptides for a linker connecting component (I) and component (II) of the GLP-1 fusion peptide.

The GLP-1 fusion peptide, which may be encoded and secreted by cells embedded in the (spherical) core of the (spherical) microcapsule, as used according to the present invention, may furthermore comprise in addition to any of the afore mentioned combinations of components of the fusion protein (i.e. components (I) and (II), components (I) and (III) or components (I), (II) and (III)) a carrier protein, in particular transferrin or albumin, as component (IV). Such a component (IV) may be linked to the N- and/or C-terminus of any of the afore mentioned combinations of components of the GLP-1 fusion protein, i.e. components (I) and/or (II), components (I) and/or (III) or components (I), (II) and/or (III), either directly or using a linker as defined herein.

In a specific embodiment of the invention, the GLP-1 (fusion) peptide as defined herein, i.e. a GLP-1 peptide or a GLP-1 fusion peptide as defined above, which may be encoded and secreted by cells embedded in the (spherical) core of the (spherical) microcapsules as used herein, contains as component (I) and/or (III) a modified GLP-1 peptide comprising the amino acid sequence of the following formula II:
Xaa7-Xaa8-Glu-Gly-Thr-Phe-Thr-Ser-Asp-Xaa16-Ser-Xaa18-Xaa19-Xaa20-Glu-Xaa22-Xaa23-Ala-Xaa25-Xaa26-Xaa27-Phe-Ile-Xaa30-Trp-Leu-Xaa33-Xaa34-Xaa35-Xaa36-Xaa37,
   wherein Xaa7 is L-histidine; Xaa8 is Ala, Gly, Val, Leu, Ile, or Lys, whereby Gly is particularly preferred; Xaa16 is Val or Leu; Xaa18 is Ser, Lys or Arg; Xaa19 is Tyr or Gln; Xaa20 is Leu or Met; Xaa22 is Gly or Glu; Xaa23 is Gln, Glu, Lys or Arg; Xaa25 is Ala or Val; Xaa26 is Lys, Glu or Arg; Xaa27 is Glu or Leu; Xaa30 is Ala, Glu or Arg; Xaa33 is Val or Lys; Xaa34 is Lys, Glu, Asn or Arg; Xaa35 is Gly; Xaa36 is Arg, Gly or Lys or amide or absent; Xaa37 is Gly, Ala, Glu, Pro, Lys, amide or is absent, wherein these amino acids are preferably selected if the GLP-1 (fusion) peptide as defined herein is encoded and secreted by cells embedded in the (spherical) core of the (spherical) microcapsules as used herein, to be administrated to a patient in need thereof, when treating an when treating AMI or MI or diseases related thereto as defined herein, or wherein Xaa7 is L-histidine, D-histidine, desamino-histidine, 2-amino-histidine, 3-hydroxy-histidine, homohistidine, N-acetyl-histidine, α-fluoromethyl-histidine, α-methyl-histidine, 3-pyridylalanine, 2-pyridylalanine or 4-pyridylalanine; Xaa8 is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid, whereby Gly is particularly preferred; Xaa16 is Val or Leu; Xaa18 is Ser, Lys or Arg; Xaa19 is Tyr or Gln; Xaa20 is Leu or Met; Xaa22 is Gly, Glu or Aib; Xaa23 is Gln, Glu, Lys or Arg; Xaa25 is Ala or Val; Xaa26 is Lys, Glu or Arg; Xaa27 is Glu or Leu; Xaa30 is Ala, Glu or Arg; Xaa33 is Val or Lys; Xaa34 is Lys, Glu, Asn or Arg; Xaa35 is Gly or Aib; Xaa36 is Arg, Gly or Lys or amide or absent; Xaa37 is Gly, Ala, Glu, Pro, Lys, amide or is absent, wherein these amino acids are preferably selected if the GLP-1 (fusion) peptide as defined herein is provided directly to a patient in need thereof, when treating AMI or MI or a diseases related thereto, as defined herein.

In still another specific embodiment of the invention component (I) and/or (III) of the GLP-1 (fusion) peptide as defined herein, i.e. a GLP-1 peptide or a GLP-1 fusion peptide as defined above, as encoded and secreted by cells embedded in the (spherical) core of the (spherical) microcapsules herein contains a modified GLP-1 peptide comprising the amino acid sequence of the following formula III:

Xaa7-Xaa8-Glu-Gly-Thr-Phe-Thr-Ser-Asp-Val-Ser-
Xaa18-Tyr-Leu-Glu-Xaa22-Xaa23-Ala-Ala-Xaa26-
Glu-Phe-Ile-Xaa30-Trp-Leu-Val-Xaa34-Xaa35-Xaa36-
Xaa37,

wherein Xaa7 is L-histidine; Xaa8 is Ala, Gly, Val, Leu, Ile, Lys; Xaa18 is Ser, Lys or Arg; Xaa22 is Gly or Glu; Xaa23 is Gln, Glu, Lys or Arg; Xaa26 is Lys, Glu or Arg; Xaa30 is Ala, Glu or Arg; Xaa34 is Lys, Glu or Arg; Xaa35 is Gly; Xaa36 is Arg or Lys, amide or is absent; Xaa37 is Gly, Ala, Glu or Lys, amide or is absent, wherein these amino acids are preferably selected if the GLP-1 (fusion) peptide as defined herein is encoded and secreted by cells embedded in the (spherical) core of the (spherical) microcapsules as used herein, to be administrated to a patient in need thereof, when treating AMI or MI or diseases related thereto as defined herein,
or wherein Xaa7 is L-histidine, D-histidine, desamino-histidine, 2-amino-histidine, -hydroxy-histidine, homohistidine, N-acetyl-histidine, α-fluoromethyl-histidine, α-methyl-histidine, 3-pyridylalanine, 2-pyridylalanine or 4-pyridylalanine; Xaa8 is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid; Xaa18 is Ser, Lys or Arg; Xaa22 is Gly, Glu or Aib; Xaa23 is Gln, Glu, Lys or Arg; Xaa26 is Lys, Glu or Arg; Xaa30 is Ala, Glu or Arg; Xaa34 is Lys, Glu or Arg; Xaa35 is Gly or Aib; Xaa36 is Arg or Lys, amide or is absent; Xaa37 is Gly, Ala, Glu or Lys, amide or is absent, wherein these amino acids are preferably selected if the GLP-1 (fusion) peptide as defined herein is provided directly to a patient in need thereof, when treating AMI or MI or a diseases related thereto, as defined herein.

In a particular preferred embodiment a GLP-1 (fusion) peptide, i.e. a GLP-1 peptide or a GLP-1 fusion peptide as defined above, is used, which may be encoded and secreted by cells embedded in the (spherical) core of the (spherical) microcapsule as used herein, wherein component (I) and/or (III) contain a (modified) GLP-1 peptide, which is selected from GLP-1 (7-35), GLP-1 (7-36), GLP-1 (7-36)-amide, GLP-1 (7-37) or a variant, analogue or derivative thereof. Also preferred are GLP-1 (fusion) peptides comprising in their components (I) and/or (III) a modified GLP-1 peptide having a Aib residue in position 8 or an amino acid residue in position 7 of said GLP-1 peptide, which is selected from the group consisting of D-histidine, desamino-histidine, 2-amino-histidine, hydroxy-histidine, homohistidine, N-acetyl-histidine, α-fluoromethyl-histidine, α-methyl-histidine, 3-pyridylalanine, 2-pyridylalanine and 4-pyridylalanine, preferably if the GLP-1 (fusion) peptide as defined herein is provided directly to a patient in need thereof, when treating AMI or MI or a diseases related thereto, as defined herein.

In another particular preferred embodiment a GLP-1 (fusion) peptide, i.e. a GLP-1 peptide or a GLP-1 fusion peptide as defined above, is used, which may be encoded and secreted by cells embedded in the (spherical) core of the (spherical) microcapsule as used herein, wherein both embodiments of components (I) and/or (III) of the GLP-1 (fusion) peptide as defined herein by formulae II and III may be combined with the disclosure given herein for GLP-1 (fusion) peptide. In other words, general formulae II and III may be combined e.g. with the disclosure given herein for component (II), linkers, process of manufacturing, etc.

A GLP-1 peptide or a GLP-1 fusion peptide as defined herein, preferably component (I) of the GLP-1 fusion peptide as defined herein, as well as their fragments and variants are preferably protected against proteolytic cleavage as outlined herein, more preferably against DPP-IV. Accordingly, such a GLP-1 peptide or a GLP-1 fusion peptide as defined herein as well as their fragments and variants, particularly GLP-1 fusion peptides, may contain a sequence of GLP-1, e.g. GLP-1(7-35, 36 or 37) (in case of GLP-1 fusion peptides as part of component (I) and/or (III)), resistant to the DPP-IV. In this context, resistance of a peptide to degradation by dipeptidyl aminopeptidase IV may be determined e.g. by the following degradation assay: Aliquots of the peptides are incubated at 37° C. with an aliquot of purified dipeptidyl aminopeptidase IV for 4-22 hours in an appropriate buffer at pH 7-8 (buffer not being albumin). Enzymatic reactions are terminated by the addition of trifluoroacetic acid, and the peptide degradation products are separated and quantified using HPLC or LC-MS analysis. One method for performing this analysis is: The mixtures are applied onto a Zorbax300SB-C18 (30 nm pores, 5 μm particles) 150×2.1 mm column and eluted at a flow rate of 0.5 ml/min with a linear gradient of acetonitrile in 0.1% trifluoroacetic acid (0%-100% acetonitrile over 30 min). Peptides and their degradation products may be monitored by their absorbance at 214 nm (peptide bonds) or 280 nm (aromatic amino acids), and are quantified by integration of their peak areas. The degradation pattern can be determined by using LC-MS where MS spectra of the separated peak can be determined. Percentage intact/degraded compound at a given time is used for estimation of the peptides DPP-IV stability.

In the herein context, a GLP-1 peptide or a GLP-1 fusion peptide as defined herein, preferably component (I) of a GLP-1 fusion peptide as defined herein, as well as a fragment and/or variant thereof, is defined as DPP-IV stabilized when it is 10 times more stable than the non-modified peptide sequence of GLP-1 (7-37) based on percentage intact compound at a given time. Thus, a DPP-IV stabilized GLP-1 peptide or GLP-1 fusion peptide, preferably component (I) of the GLP-1 fusion peptide as defined herein, is preferably at least 10, more preferably at least 20 times more stable than e.g. GLP-1 (7-37). Stability may be assessed by any method known to the skilled person, e.g. by adding DPP-IV to a solution of the peptide to be tested and by determining the degradation of the peptide (see herein), e.g. over a period of time, by e.g. a spectroscopic method, Western-Blot analysis, antibody screening etc.

In parallel, a GLP-1 peptide or GLP-1 fusion peptide, preferably component (I) of a GLP-1 fusion peptide as defined herein, as well as a fragment and/or variant thereof is defined as a compound, which exerts the effect of GLP-1(7-37) by e.g. binding to its native receptor (GLP-1 receptor). Preferably, a GLP-1 (fusion) peptide or a GLP-1 fusion peptide, as well as a fragment and/or variant thereof as defined herein has a binding affinity to the GLP-1 receptor, which corresponds to at least 10%, preferably at least 50% of the binding affinity of the naturally occurring GLP-1 peptide. The binding affinity may be determined by any suitable method, e.g. surface plasmon resonance, etc. Moreover, it is preferred, if the GLP-1 (fusion) peptide or GLP-1 fusion peptide, as well as a fragment and/or variant thereof as defined herein, evokes formation of intracellular cAMP by its binding to its extracellular receptor, which transmits the signal into the cell.

According to another preferred embodiment, the GLP-1 peptide or GLP-1 fusion peptide, preferably as defined herein, as well as the single components of the GLP-1 fusion peptide, particularly components (I), (II) and (III), and/or the entire GLP-1 fusion peptide as described herein, may be selected from modified forms of these peptides or proteins sequences. The various modified forms, particularly a modified form of the entire GLP-1 fusion peptide as described herein, may be either encoded and secreted by cells embedded in the (spherical) core of the (spherical) microcapsule as used herein or may be used directly in the treatment of AMI or MI or diseases related thereto. These modified forms are disclosed in the following and described in more detail and comprise e.g. fragments, variants, etc., of the GLP-1 peptide, preferably as defined herein or of single components of the GLP-1 fusion peptide, particularly components (I), (II) and (III), and/or the entire GLP-1 fusion peptide as described herein. In this context, fragments and/or variants of these peptides or proteins may have a sequence identity to their native peptides or proteins of at least 40%, 50%, 60%, 70%, 80%, preferably at least 90%, more preferably at least 95% and most preferably at least 99% over the whole length of the native, non-modified amino acid sequence. This likewise may be applied to the respective (coding) nucleic acid sequence.

The term “sequence identity” as defined herein typically means that the sequences are compared as follows. To determine the percent identity of two amino acid sequences, the sequences can be aligned for optimal comparison purposes (e.g., gaps can be introduced in the sequence of a first amino acid sequence). The amino acids at corresponding amino acid positions can then be compared. When a position in the first sequence is occupied by the same amino acid as the corresponding position in the second sequence, then the molecules are identical at that position. The percent identity between the two sequences is a function of the number of identical positions shared by the sequences, e.g. where a particular peptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide, which is 50% identical to the reference polypeptide over its entire length. Of course, other polypeptides will meet the same criteria. Such a determination of percent identity of two sequences can be accomplished using a mathematical algorithm. A preferred, non-limiting example of a mathematical algorithm utilized for the comparison of two sequences is the algorithm of Karlin et al. (1993), PNAS USA, 90:5873-5877. Such an algorithm is incorporated into the NBLAST program, which can be used to identify sequences having the desired identity to the amino acid sequence of the invention. To obtain gapped alignments for comparison purposes, Gapped BLAST can be utilized as described in Altschul et al. (1997), Nucleic Acids Res, 25:3389-3402. When utilizing BLAST and Gapped BLAST programs, the default parameters of the respective programs (e.g., NBLAST) can be used. The sequences further may be aligned using Version 9 of the Genetic Computing Group's GAP (global alignment program), using the default (BLOSUM62) matrix (values-4 to +11) with a gap open penalty of −12 (for the first null of a gap) and a gap extension penalty of −4 (per each additional consecutive null in the gap). After alignment, percentage identity is calculated by expressing the number of matches as a percentage of the number of amino acids in the claimed sequence. The described methods of determination of the percent identity of two amino acid sequences can be applied correspondingly to nucleic acid sequences. In the context of the present invention, the term “identity” is used, however, the term “homology” may also be applied instead of the term “identity”, wherever necessary or desired.

In the context of the present invention, a “fragment” of a GLP-1 peptide, preferably as defined herein or of single components of the GLP-1 fusion peptide, particularly components (I), (II) and (III), and/or the entire GLP-1 fusion peptide as described herein, typically refers to any fragment of these peptides or proteins. Typically, such a fragment comprises a shorter peptide which retains the desired biological activity particularly of the native peptide or protein, which is, with regard to its amino acid sequence (or its encoded nucleic acid sequence), N-terminally, C-terminally and/or intrasequentially truncated compared to the amino acid sequence of the native peptide or protein (or its encoded nucleic acid sequence). Such truncation may thus occur either on the amino acid level or correspondingly on the nucleic acid level. Biologically functional fragments may be readily identified by removing amino acids (either on peptide or on amino acid level) from either end of the peptide molecule and testing the resultant peptide or protein for its biological properties as defined herein for GLP-1. Proteases for removing one or more amino acids at a time from either the N-terminal end and/or the C-terminal end of a native peptide or protein may be used to determine fragments which retain the desired biological activity. Conclusively, fragments may be due to deletions of amino acids at the peptide termini and/or of amino acids positioned within the peptide sequence.

Furthermore, a “variant” of a GLP-1 peptide, preferably as defined herein or of single components of the GLP-1 fusion peptide, particularly components (I), (II) and (III), and/or the entire GLP-1 fusion peptide as described herein, preferably comprises a protein sequence or its encoding nucleic acid sequence (or a fragment thereof), wherein amino acids of the native protein or peptide sequences are exchanged. Thereby, (a variant of) a GLP-1 peptide, preferably as defined herein, a GLP-1 fusion peptide, or of single components of the GLP-1 fusion peptide, particularly components (I), (II) and (III), and/or the entire GLP-1 fusion peptide as described herein may be generated, having an amino acid sequence which differs from the native protein or peptide sequences in one or more mutation(s), such as one or more substituted, inserted and/or deleted amino acid(s). Preferably, these variants have about the same or an improved biological activity as defined herein for GLP-1, be it a variant of GLP-1, a GLP-1 fusion peptide itself or a functional variant and/or fragment thereof, i.e. the beneficial effects known for GLP-1, e.g. its activity to powerfully reduce the damages caused by ischemia or oxygen shortage and potential death of heart tissue compared to the full-length GLP-1 peptide, GLP-1 fusion peptide or full-length single components of the GLP-1 fusion peptide, particularly components (I), (II) and (III).

Such a variant as defined herein can be prepared by mutations in the DNA sequence which encodes the synthesized variants. Any combination of deletion, insertion, and substitution may also be contained in GLP-1 peptides encoded and secreted by a cell as embedded in the (spherical) microcapsule as defined herein, provided that the finally obtained variant possesses the desired biological activity. Obviously, the mutations that will be made in the DNA encoding the variant peptide must not alter the reading frame and preferably will not create complementary regions that could produce secondary mRNA structure.

Accordingly, a variant of a GLP-1 peptide, preferably as defined herein, a GLP-1 fusion peptide, or of single components of the GLP-1 fusion peptide, particularly components (I), (II) and (III), and/or the entire GLP-1 fusion peptide as described herein, may also contain additional amino acid residues flanking the N-/or the C-terminus or even both termini of the amino acid sequence compared to the native GLP-1 peptide or native GLP-1 fusion peptide as described herein. As an example, such a variant may comprise a GLP-1 peptide or a GLP-1 fusion peptide as defined herein containing additional amino acid residues flanking the N-1 or the C-terminus or even both termini of the amino acid sequence of the GLP-1 peptide or GLP-1 fusion peptide. As long as the resultant GLP-1 peptide or GLP-1 fusion peptide retains its resistance or stability towards proteases and its ability to act as defined herein, one can determine whether any such flanking residues affect the basic characteristics of the “core” peptide, e.g. by its beneficial effects known for GLP-1, by routine experimentation. The term “consisting essentially of”, when referring to a specified GLP-1 peptide as defined herein, means that additional flanking residues can be present which do not affect the basic characteristic of the specified GLP-1 peptide. This term typically does not comprehend substitutions, deletions or additions within the specified sequence.

A “variant” of a GLP-1 peptide, preferably as defined herein, a GLP-1 fusion peptide, or of single components of the GLP-1 fusion peptide, particularly components (I), (II) and (III), and/or the entire GLP-1 fusion peptide as described herein, may further refer to a molecule which comprises conservative amino acid substitutions compared to its native sequence. Substitutions in which amino acids which originate from the same class are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain. Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

A variant of a GLP-1 peptide, preferably as defined herein, a GLP-1 fusion peptide, or of single components of the GLP-1 fusion peptide, particularly components (I), (II) and (III), and/or the entire GLP-1 fusion peptide as described herein may thus also refer to a molecule which is substantially similar to either the entire GLP-1 peptide, preferably as defined herein, the entire GLP-1 fusion peptide, or to single components of the GLP-1 fusion peptide, particularly components (I), (II) and (III), or a fragment thereof. Such variant peptides may be conveniently prepared using methods well known in the art. Of course, such a variant would have similar beneficial effects known for the native GLP-1 peptide, preferably as defined herein, a GLP-1 fusion peptide, or of single components of the GLP-1 fusion peptide, particularly components (I), (II) and (III), and/or the entire GLP-1 fusion peptide as described herein. Such beneficial effect is, e.g. for GLP-1, its activity to powerfully reduce the damages caused by ischemia or oxygen shortage and potential death of heart tissue as the corresponding naturally-occurring GLP-1 peptide.

The types of conservative amino acid substitutions which may be contained in a variant of the GLP-1 peptide, preferably as defined herein, a GLP-1 fusion peptide, or of single components of the GLP-1 fusion peptide, particularly components (I), (II) and (III), and/or the entire GLP-1 fusion peptide as described herein, may be based on analysis of the frequencies of amino acid changes between a homologous protein/peptide of different species. Based upon such analysis, conservative substitutions may be defined herein as exchanges within one of the following five groups:
I. Small, aliphatic, non-polar or slightly polar residues: Ala, Ser, Thr, Pro, Gly;
II. Polar, negatively-charged residues and their amides: Asp, Asn, Glu, Gln;
III. Polar, positively-charged residues: His, Arg, Lys;
IV. Large, aliphatic non-polar residues: Met, Leu, Ile, Val, Cys;
V. Large aromatic residues: Phe, Try, Trp.

Within the foregoing groups, the following substitutions are considered to be “highly conservative”: Asp/Glu; His/Arg/Lys; Phe/Tyr/Trp; Met/Leu/Ile/Val. Semi-conservative substitutions are defined to be exchanges between two of groups (I)-(IV) herein which are limited to supergroup (A), comprising (I), (II), and (III) herein, or to supergroup (B), comprising (IV) and (V) herein. Substitutions are not limited to the genetically encoded or even the naturally-occurring amino acids. Preferred conservative amino acid substitutions of preferred groups of synonymous amino acid residues within the herein meaning particularly include, without being limited thereto:

| Amino Acid | Synonymous Residue |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, (Thr), Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, (Thr), Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, (Thr), Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

Furthermore, variants of a GLP-1 peptide, preferably as defined herein, a GLP-1 fusion peptide, or of single components of the GLP-1 fusion peptide, particularly components (I), (II) and (III), and/or the entire GLP-1 fusion peptide as described herein, may also contain amino acid substitutions, made e.g. with the intention of improving solubility (replacement of hydrophobic amino acids with hydrophilic amino acids).

In one particularly preferred embodiment a GLP-1 peptide or a GLP-1 fusion peptide as defined herein, which may be encoded and secreted by a cell embedded in the (spherical) core of the (spherical) microcapsule as defined herein, includes a GLP-1 peptide (occurring in component (I) and/or (III) of the GLP-1 fusion peptide) characterized by one or more substitution(s) at positions 7, 8, 11, 12, 16, 22, 23, 24, 25, 27, 30, 33, 34, 35, 36, or 37 of the GLP-1 peptide. As an example for the following nomenclature [Arg34-GLP-1 (7-37)] designates a GLP-1 analogue wherein its naturally occurring lysine at position 34 has been substituted with arginine.

Specifically, a GLP-1 peptide or component (I) and/or (III) of a GLP-1 fusion peptide as defined herein may correspond to variants of GLP-1(7-35, 36, 37 or 38) including, for example, Gln9-GLP-1 (7-37), Thr16-Lys18-GLP-1 (7-37), and Lys18-GLP-1 (7-37), Arg34-GLP-1 (7-37), Lys38-Arg26-GLP-1 (7-38)-OH, Lys36-Arg26-GLP-1 (7-36), Arg26,34-Lys38-GLP-1 (7-38), Arg26,34-Lys38-GLP-1(7-38), Arg26,34-Lys38-GLP-1 (7-38), Arg26,34-Lys38-GLP-1 (7-38), Arg26,34-Lys38-GLP-1 (7-38), Arg26-Lys38-GLP-1(7-38), Arg26-Lys38-GLP-1 (7-38), Arg34-Lys38-GLP-1 (7-38), Ala37-Lys38-GLP-1 (7-38), and Lys37-GLP-1 (7-37). More generally speaking, any GLP-1 variant mentioned herein (in particular according to formulae II or III) may be modified by the addition of a Lys residue at position 38.

If the GLP-1 peptide or GLP-1 fusion peptide as described herein is administered directly in the treatment of AMI or MI or diseases related thereto, the GLP-1 peptide or component (I) and/or (III) of a GLP-1 fusion peptide as defined herein may additionally correspond to variants of GLP-1(7-35, 36, 37 or 38) including Gln9-GLP-1 (7-37), D-Gln9-GLP-1(7-37), acetyl-Lys9-GLP-1 (7-37).

In a particular preferred embodiment of the invention the GLP-1 peptide or the GLP-1 fusion peptide as defined herein (with respect to component (I) or (III)) is/contains a (modified) GLP-1 peptide, which is selected from GLP-1 (7-35), GLP-1 (7-36), GLP-1 (7-36)-amide, GLP-1 (7-37) or a fragment or variant thereof.

For in vitro control purposes the GLP-1 peptide or GLP-1 fusion peptide as defined herein may be isolated from the cells (and thus from the miocrocapsules) from which it is expressed, for instance using conventional separation techniques. Thus cells may be grown under appropriate conditions, for instance including support and nutrients, in vitro, and secreted protein, i.e. the GLP-1 peptide or GLP-1 fusion peptide as defined herein, if encoded and secreted by a cell embedded in the (spherical) core of the (spherical) microcapsule or a fragment or variant thereof, is recovered from the extracellular medium. The (vector) sequences engineered for transfection into cells thus preferably include signal (peptide) sequences (see below) allowing secretion of the GLP-1 peptide or GLP-1 fusion peptide as defined herein. In this context, the GLP-1 peptide or GLP-1 fusion peptide as defined herein, if encoded and secreted by a cell embedded in the (spherical) core of the (spherical) microcapsule, or a fragment or variant thereof, may be fused to a signal sequence, either naturally endogenously or after transfection of encoding nucleic acid sequences introduced into the cell by genetic engineering methods. In an alternative, the engineered gene sequences encoding a GLP-1 peptide as defined herein do not include such signal peptide sequences, whereby the intracellularly expressed GLP-1 peptides or GLP-1 fusion peptides will typically not be secreted, and may be recovered from cells by processes involving cell lysis. In such methods the coding sequences may include purification tags allowing efficient extraction of the product peptide from the medium; tags may be cleaved off to release isolated GLP-1 peptide. However, this alternative is typically irrelevant to cells of a (spherical) microcapsule, as used according to the present invention, which are implanted into the patient and require delivery of an in vivo expressed and secreted GLP-1 peptide or GLP-1 fusion peptide as defined herein into the surrounding tissue.

Any of the herein described embodiments or features may be combined with each other, if not indicated otherwise.

The cells embedded in the (spherical) core of the (spherical) microcapsule used according to the present invention preferably encode and secrete the GLP-1 peptide or GLP-1 fusion peptide as defined herein, and optionally an additional factor, such as an anti-apoptotic agent, VEGF, etc. as defined herein. For these purposes, the GLP-1 peptide or GLP-1 fusion peptide as defined herein or its fragments or variants as well as further additional factors, are encoded by at least one nucleic acid sequence, which is typically transfected into the cells prior to preparation of the (spherical) core of the (spherical) microcapsule. These nucleic acid sequences may occur naturally in the cells or may be introduced into the cells by cell transfection techniques prior to the preparation of the (spherical) microcapsule. According to the present invention any suitable nucleic acid sequence coding for a GLP-1 peptide as defined herein may be used.

According to one embodiment, the nucleic acid sequence encoding the GLP-1 peptide or GLP-1 fusion peptide as defined herein, or a fragment or variant thereof, and optionally an additional factor, such as an anti-apoptotic agent, VEGF, etc. as defined herein may be selected from any nucleic acid, more preferably selected from any nucleic acid suitable to encode (at least one) peptide or protein, i.e. a coding nucleic acid, e.g. a coding DNA, selected e.g. from genomic DNA, cDNA, DNA oligonucleotides, or a coding RNA, selected e.g. from (short) RNA oligonucleotides, messenger RNA (mRNA), etc. In the context of the present invention, an mRNA is typically an RNA, which is composed of several structural elements, e.g. an optional 5′-UTR region, an upstream positioned ribosomal binding site followed by a coding region, an optional 3′-UTR region, which may be followed by a poly-A tail (and/or a poly-C-tail). An mRNA may occur as a mono-, di-, or even multicistronic RNA, i.e. an RNA which carries the coding sequences of one, two or more proteins or peptides as described herein. Such coding sequences in di-, or even multicistronic mRNA may be separated by at least one IRES sequence. The least one nucleic acid sequence may also be a ribosomal RNA (rRNA), a transfer RNA (tRNA), or a viral RNA (vRNA). Furthermore, the least one nucleic acid sequence may be a circular or linear nucleic acid, preferably a linear nucleic acid. Additionally, the at least one nucleic acid sequence may be a single- or a double-stranded nucleic acid sequence (which may also be regarded as a nucleic acid within the herein meaning due to non-covalent association of two single-stranded nucleic acids) or a partially double-stranded or partially single stranded nucleic acid, which are at least partially self complementary (both of these partially double-stranded or partially single stranded nucleic acids are typically formed by a longer and a shorter single-stranded nucleic acid or by two single stranded nucleic acids, which are about equal in length, wherein one single-stranded nucleic acid is in part complementary to the other single-stranded nucleic acid and both thus form a double-stranded nucleic acid in this region, i.e. a partially double-stranded or partially single stranded nucleic acid).

Due to degeneracy of the genetic code a plurality of nucleic acid sequences may code for such a GLP-1 peptide or GLP-1 fusion peptide as defined herein, and optionally an additional factor, such as an anti-apoptotic agent, VEGF, etc. as defined herein. According to a preferred embodiment of the present invention a nucleic acid sequence used for transfection of cells as defined herein may comprise any nucleic acid sequence coding for the GLP-1 peptide or GLP-1 fusion peptide as defined herein as defined herein and additional (functional) nucleotide sequences. The present invention, preferably a nucleic acid sequence suitable for transfection of a cell as defined herein which may code for (a) the GLP-1 peptide or GLP-1 fusion peptide as defined herein, particularly for the entire GLP-1 aa sequence (GLP-1(1-37) or functional GLP-1(7-35, 36 or 37) (variant) sequences or any other GLP-1 peptide, including GLP-1 fusion peptides as defined herein, (b) optionally for a protease cleavage sequence at the N-terminus of the GLP-1 sequence according to (a) and, optionally, for a signal peptide sequence upstream from (b), and (c) optionally for a further factor as described herein. Preferably, the signal (peptide) sequence is selected from a sequence as defined below. Accordingly, the resulting amino acid sequence may be composed of a signal peptide sequence, an optional protease cleavage sequence and the GLP-1 peptide or GLP-1 fusion peptide as defined herein, or a fragment or variant thereof (, and optionally an additional factor, such as an anti-apoptotic agent, VEGF, etc. as defined herein), (preferably from the N- to the C-terminus). Thereby, the signal peptide sequence and the protease cleavage sequence are preferably heterologous to (the natively occurring sequences in the) host cell, and are, in case of GLP-1(5-37, 6-37, or 7-37) and variants thereof as defined herein preferably different from the amino acids 1 to 6 of native GLP-1 within the definitions of the herein proviso.

The nucleic acid sequence as defined herein may be contained in a vector. Accordingly, the cell embedded in the (spherical) core of the (spherical) microcapsule used according to the present invention may contain a vector comprising a nucleic acid as defined herein before. This vector may be used to transfect the cell as defined herein to prepare the (spherical) microcapsule as used according to the present invention. Typically, such a vector, in particular an expression vector, contains at least one nucleic acid sequence as defined herein, encoding elements (a) and optionally (b) and/or (c) as described herein, and, if necessary, additional elements as described herein, e.g. elements suitable for directing expression of the encoded elements (a) and optionally (b) and/or (c) as described herein, and optionally sequences encoding further factors, such as antiapoptotoc factors, VEGF, etc. One class of vectors as used herein utilizes DNA elements that provide autonomously replicating extrachromosomal plasmids derived from animal viruses (e.g. bovine papilloma virus, polyomavirus, adenovirus, or SV40, etc.). A second class of vectors as used herein relies upon the integration of the desired gene sequences into the host cell chromosome.

Such vectors, suitable to transfect the cell prior to embedding it in the (spherical) core of the (spherical) microcapsule used according to the present invention, are typically prepared by inserting at least one nucleic acid sequence encoding elements (a) and optionally (b) and/or (c) as described herein, e.g. the GLP-1 peptide or GLP-1 fusion peptide as defined herein, or a fragment or variant thereof, and optionally an additional factor as defined herein into suitable (empty) vectors. Such suitable (empty) vectors are known to a skilled person and may be reviewed e.g. in “Cloning Vectors” (Eds. Pouwels P. H. et al. Elsevier, Amsterdam-New York-Oxford, 1985, ISBN 0 444 904018). Suitable (empty) vectors are also intended to include any vector known to a skilled person, such as plasmids, phages, viruses such as SV40, CMV, Baculo virus, Adeno virus, Sindbis virus, transposons, IS-elements, phasmids, phagemides, cosmides, linear or circular DNA. For integration in mammalian cells linear DNA is typically used. Preferably, the vector type used for the present invention corresponds to the specific host cell requirements. Suitable commercially available expression vectors, into which the inventive nucleic acid sequences and/or vectors may be inserted, include pSPORT, pBluescriptllSK, the baculovirus expression vector pBlueBac, and the prokaryotic expression vector pcDNAII, all of which may be obtained from Invitrogen Corp., San Diego, Calif.

A vector as defined herein suitable for transfecting a cell prior to embedding it in the (spherical) core of the (spherical) microcapsule used according to the present invention, typically combines the nucleic acid sequence as defined herein with other regulatory elements, which, e.g., control expression of the encoded amino acid sequences. Such regulatory elements are e.g. 1) specific to a tissue or region of the body; 2) constitutive; 3) glucose responsive; and/or 4) inducible/regulatable. Regulatory elements herein are preferably selected from regulation sequences and origins of replication (if the vectors are replicated autonomously). Regulation sequences in the scope of the present invention are any elements known to a skilled person having an impact on expression on transcription and/or translation of the encoding nucleic acid sequences. Regulation sequences include, apart from promoter sequences so-called enhancer sequences, which may lead to an increased expression due to enhanced interaction between RNA polymerase and DNA. Further regulation sequences of inventive vectors are transcriptional regulatory and translational initiation signals, so-called “terminator sequences”, etc. or partial sequences thereof.

Generally, any naturally occurring promoter may be contained in an expression vector suitable for transfecting a cell which may be used for preparing the (spherical) microcapsule as used herein. Such promoters may be selected from any eukaryotic, prokaryotic, viral, bacterial, plant, human or animal, e.g. mammalian promoters. Suitable promoters include, for example, the cytomegalovirus promoter, the lacZ promoter, the gal 10 promoter and the AcMNPV polyhedral promoter, promoters such as cos-, tac-, trp-, tet-, trp-tet-, Ipp-, lac-, lpp-lac-, laclq-, T7-, T5-, T3-, gal-, trc-, ara-, SV40-, SP6, I-PR- or the I-PL-promoter, advantageously being found in gram-negative bacteria. Additionally, promoters may be obtained from gram-positive promoters such as amy and SPO₂, yeast promoters, such as ADC1, MFa, AC, P-60, CYC1, GAPDH or mammalian promoters such as the cytomegalovirus (CMV) promoter, muscle-specific promoters including mammalian muscle creatine kinase (MCK) promoter, mammalian desmin promoter, mammalian troponin I (TNNI2) promoter, or mammalian skeletal alpha-actin (ASKA) promoter, or liver type pyruvate kinase promoters, particularly those fragments which run (−183 to +12) or (−96 to +12) (Thompson, et al. J Biol Chem, (1991). 266:8679-82.; Cuif, et al, Mol Cell Biol, (1992). 12:4852-61); the spot 14 promoter (S14, −290 to +18) (Jump, et al, J. Biol Chem, (1990). 265:3474-8); acetyl-CoA carboxylase (O'Callaghan, et al., J. Biol Chem, (2001). 276:16033-9); fatty acid synthase (−600 to +65) (Rufo, et al, J Biol Chem, (2001). 28:28); and glucose-6-phosphatase (rat and human) (Schmoll, et al, FEBS Left, (1996). 383:63-6; Argaud, et al, Diabetes, (1996). 45:1563-71), or promoters from CaM-Kinasell, Nestin, L7, BDNF, NF, MBP, NSE, beta-globin, GFAP, GAP43, tyrosine hydroxylase, Kainat-receptor-subunit 1, glutamate-receptor-subunit B, or human ubiquitin promoter B (ubiB human), human ferritin H promoter (FerH), etc. Particularly preferred promoters are of human or mammalian origin. Finally, synthetic promoters may be used advantageously. Promoter sequences, as contained in an inventive vector, may also be inducible for in vitro control purposes, to allow modulation of expression (e.g. by the presence or absence of nutrients or other inducers in the growth medium). One example is the lac operon obtained from bacteriophage lambda plac5, which can be induced by IPTG. Finally, a promoter as defined herein may be linked with a GLP-1 encoding nucleic acid sequence as defined herein, and optionally an additional factor, such as an anti-apoptotic agent, VEGF, etc. as defined herein, such that the promoter is positioned 5′ “upstream” of the GLP-1 encoding nucleic acid sequence. Preferably, human promoters are used, e.g. the human ubiquitin promoter B (ubiB human) or the human ferritin H promoter (FerH).

Enhancer sequences for upregulating expression of GLP-1 encoding nucleic acid sequences as defined herein are preferably another constituent of a vector or an expression as defined herein. Such enhancer sequences are typically located in the non-coding 3′ region of the vector. Enhancer sequences as employed in a vector as defined herein may be obtained from any eukaryotic, prokaryotic, viral, bacterial, plant, human or animal, e.g. mammalian hosts, preferably in association with the corresponding promoters as defined herein. Enhancer elements which will be most useful in the present invention are those which are glucose responsive, insulin responsive and/or liver specific. Enhancer elements may include the CMV enhancer (e.g., linked to the ubiquitin promoter (Cubi)); one or more glucose responsive elements, including the glucose responsive element (G1RE) of the liver pyruvate kinase (L-PK) promoter (−172 to −142); and modified versions with enhanced responsiveness (Cuif et al, supra; Lou, et al, J. Biol Chem, (1999). 274:28385-94); G1RE of L-PK with auxiliary L3 box (−172 to −126) (Diaz Guerra, et al, Mol Cell Biol, (1993). 13:7725-33; modified versions of G1RE with enhanced responsiveness with the auxiliary L3 box; carbohydrate responsive element (ChoRE) of S 14 (−1448 to −1422), and modifications activated at lower glucose concentrations (Shih and Towle, J Biol Chem, (1994). 269:9380-7; Shih, et al, J Biol Chem, (1995). 270:21991-7; and Kaytor, et al, J Biol Chem, (1997). 272:7525-31; ChoRE with adjacent accessory factor site of S 14 (−1467 to −1422) [et al, supra]; aldolase (+1916 to +2329) (Gregori et al, J Biol Chem, (1998). 273:25237-43; Sabourin, et al, J. Biol Chem, (1996). 271:3469-73; and fatty acid synthase (−7382 to −6970) (Rufo, et al, supra.), more preferably insulin responsive elements such as glucose-6-phosphatase insulin responsive element (−780 to −722) [Ayala et al., Diabetes, (1999). 48:1885-9; and liver specific enhancer elements, such as prothrombin (940 to −860) [Chow et al, J Biol Chem, (1991) 266: 18927-33; and alpha-1-microglobulin (−2945 to −2539) [Rouet et al, Biochem J, (1998). 334:577-84), Muscle-specific enhancers such as mammalian MCK enhancer, mammalian DES enhancer, and vertebrate troponin I IRE (TNI IRE, herein after referred to as FIRE) enhancer. Finally, a SV40 enhancer sequence may also be included.

Enhancer elements may further be used along with promoters as defined herein for upregulating expression of GLP-1 encoding nucleic acid sequences as defined herein, e.g. such promoter/enhancer combinations include e.g. the cytomegalovirus (CMV) promoter and the CMV enhancer, the CMV enhancer linked to the ubiquitin promoter (Cubi), the group of liver-specific enhancer elements comprising human serum albumin [HSA] enhancers, human prothrombin [HPrT] enhancers, alpha-1 microglobulin [A1MB] enhancers, and intronic aldolase enhancers used in combination with their corresponding promoters, or HSA enhancers used in combination with a promoter selected from the group of a CMV promoter or an HSA promoter, enhancer elements selected from the group consisting of human prothrombin [HPrT] and alpha-1 microglobulin [A1MB] used in combination with the CMV promoter enhancer elements selected from the group consisting of human prothrombin [HPrT] and alpha-1 microglobulin [A1MB] used in combination with the alpha-1-anti trypsin promoter, etc.

Furthermore, a vector as defined herein suitable for transfecting a cell which may be used as constituent of the (spherical) microcapsule as used according to the present invention, may contain transcriptional and/or translational signals, preferably transcriptional and/or translational signals recognized by an appropriate host, such as transcriptional regulatory and translational initiation signals. Transcriptional and/or translational signals may be obtained from any eukaryotic, prokaryotic, viral, bacterial, plant, preferably human or animal, e.g. mammalian hosts, preferably in association with the corresponding promoters as defined herein. A wide variety of transcriptional and translational regulatory sequences may be employed therefore, depending upon the nature of the host to the extent that the host cells recognizes the transcriptional regulatory and translational initiation signals associated with a GLP-1 encoding nucleic acid sequence, and optionally an additional factor as defined herein. The 5′ region adjacent to the naturally occurring GLP-1 encoding nucleic acid sequence may be retained and employed for transcriptional and translational regulation in an inventive vector. This region typically will include those sequences involved with initiation of transcription and translation, such as the TATA box, capping sequence, CAAT sequence, and the like. Typically, this region will be at least about 150 base pairs long, more typically about 200 bp, and rarely exceeding about 1 to 2 kb.

Transcriptional initiation regulatory signals suitable for a vector as defined herein may be selected that allow to control repression or activation such that expression of the GLP-1 encoding or nucleic acid sequences as defined herein, and optionally of an additional factor as defined herein, can be modulated. One such controllable modulation technique is the use of regulatory signals that are temperature-sensitive in order to repress or initiate expression by changing the temperature. Another controllable modulation technique is the use of regulatory signals that are sensitive to certain chemicals. These methods are preferably to be used in in vitro procedures, e.g. when preparing the necessary constructs.

Furthermore, transcriptional initiation regulatory signals may be use herein, which allow control repression or activation of expression in vivo without any further means from outside the cell, e.g. to obtain a transient expression in the encapsulated cells. Such transcription and/or translational signals include e.g. transcriptional termination regulatory sequences, such as a stop signal and a polyadenylated region. Furthermore, transcriptional termination regulatory sequences may be located in the non-coding 3′ region of a vector as defined herein containing the GLP-1 encoding nucleic acid sequence. Suitable termination sequences can include, for example, the bovine growth hormone, SV40, lacZ, EF1 alpha and AcMNPV polyhedral polyadenylation signals.

The expression vectors suitable for transfecting a cell which may be used for preparing the (spherical) microcapsule as used according to the present invention, may also include other sequences for optimal expression of the GLP-1 encoding or nucleic acid sequences as defined herein, and optionally of an additional factor as defined herein. Such sequences include those encoding signal (peptide) sequences, i.e. which encode N-terminally located peptide sequences that provide for passage of the secreted protein into or through a membrane; which provide for stability of the expression product; and restriction enzyme recognition sequences, which provide sites for cleavage by restriction endonucleases. All of these materials are known in the art and are commercially available (see, for example, Okayama (1983), Mol. Cell. Biol., 3: 280).

As defined herein “a signal sequence” is a signal (peptide) sequence which typically comprises about 8 to 30 amino acids, or 15 to 30 mino acids, located—within the definitions of the herein proviso regarding amino acids 1 to 6 of GLP-1—at the N-terminus of the expressed GLP-1 (fusion) peptide and enables the GLP-1 peptide to be secreted, i.e. to pass through a cell membrane. Such a signal (peptide) sequence may include the signal sequence normally associated with the wild type GLP-1 precursor protein (i.e., the signal sequence(s) of the full length proglucagon precursor molecule), as well as signal (peptide) sequences which are not normally associated thereto, i.e. which are heterologous to the wild type GLP-1 precursor protein (i.e., the signal (peptide) sequence(s) of the full length proglucagon precursor molecule). A “signal (peptide) sequence” as defined herein can be, for example, a signal peptide sequence or a leader sequence (e.g. a secretory signal (and leader) sequence). Furthermore, signal (peptide) sequences as defined herein preferably provide for cleavage of the (GLP-1) precursor peptide by a protease, e.g. a signal (peptide) sequence protease. Upon cleavage of the signal (peptide) sequence from the (GLP-1) precursor peptide by the protease a biologically active GLP-1 peptide as defined herein is produced. Such a signal (peptide) sequence generally comprises a region which encodes a cleavage site recognized by a protease for cleavage. Alternatively, a region which encodes a cleavage site recognized by a protease for cleavage can be introduced into the signal (peptide) sequence. Furthermore, additional (one or more) sequences which encodes a cleavage site recognized by a protease for cleavage can be added to the signal (peptide) sequence.

Examples of signal (peptide) sequences which can be encoded by a vector as defined herein include a signal (peptide) sequence derived from a secreted protein such as GLP-1 or other than GLP-1, such as a cytokine, a clotting factor, an immunoglobulin, a secretory enzyme or a hormone (including the pituitary adenylate cyclase activating polypeptide (PACAP)/glucagon superfamily) and a serum protein. For example, a signal (peptide) sequence as defined herein can be derived from secreted matrix metalloproteinases (MMP), e.g. a stromelysin leader sequence, from secreted human alkaline phosphatase (SEAP), pro-exendin, e.g. a proexendin-4 leader sequence, pro-helodermin, pro-glucose-dependent insulinotropic polypeptide (GIP), pro-insulin-like growth factor (IGF1), preproglucagon, alpha-1 antitrypsin, insulin-like growth factor 1, human factor IX, human lymphotoxin A (Genbank Accession no. BAA00064), or human clusterin (Genbank Accession No. AAP88927). Particular examples of signal (peptide) sequences as defined herein are sequences which include a coding region for a signal for precursor cleavage by signal peptidase, furin or other prohormone convertases (e.g., PC3). For example, a signal (peptide) sequence which is cleaved by furin (also known as PACE, see U.S. Pat. No. 5,460,950), other subtilisins (including PC2, PC1/PC3, PACE4, PC4, PC5/PC6, LPC/PC7IPC8/SPC7 and SKI-1; Nakayama, Biochem. J., 327:625-635 (1997)); enterokinase (see U.S. Pat. No. 5,270,181) or chymotrypsin can be introduced into the signal (peptide) sequence as defined herein. The disclosure of each of these documents is hereby incorporated herein by reference. Furin is a ubiquitously expressed protease that resides in the trans-golgi and processes protein precursors before their secretion. Furin cleaves at the COOH-terminus of its consensus recognition sequence, Arg-X-Lys-Arg or Arg-X-Arg-Arg, (Lys/Arg)-Arg-X-(Lys/Arg)-Arg and Arg-X-X-Arg, such as an Arg-Gln-Lys-Arg. These amino acid sequences are a signal for precursor cleavage by the protease furin. Thus, a heterologous signal (peptide) sequence can also be synthetically derived from a consensus sequence compiled from signal (peptide) sequences (e.g., a consensus sequence compiled from secreted proteins that are cleaved by signal peptidase).

Additionally to regulation sequences as defined herein, an autonomously replicating vector as defined herein typically comprises an origin of replication. Suitable origins of replication include, without being limited thereto, e.g. ColE1, pSC101, SV40, pMPI (ori pMPI) and M13 origins of replication, etc.

Preferably, a vector as defined herein, suitable for expression of the GLP-1 encoding nucleic acid sequences of the cells of the (spherical) microcapsules as defined herein, and optionally of an additional factor as defined herein, may additionally contain a suicide gene. In the context of the present invention “a suicide gene” is preferably capable to stop the therapy with (spherical) microcapsules, as used herein, by killing the suicide gene harbouring cell contained in the (spherical) core of the (spherical) microcapsule upon administering a specific substance. In other words, a suicide gene suitable for the present invention may be activated by administering an exogenous activator that typically does not occur in the human or animal body. In this case, typically the suicide gene initiates a cascade causing the cell to undergo an apoptotic event. Alternatively, a suicide gene suitable for the present invention may metabolize an administered exogenous non-toxic prodrug that typically does not occur in the human or animal body. Metabolism of the exogenous non-toxic prodrug preferably renders the prodrug to a cell toxin. The suicide gene may be contained on the same vector encoding the GLP-1 peptide of GLP-1 fusion peptide as defined herein or alternatively on a second vector. Furthermore, the suicide gene may be regulated by control and regulatory elements of any kind, e.g. control and regulatory elements such as promoters, enhancers, etc. as mentioned herein as constituents of expression vectors, or by their naturally occurring control and regulatory elements. Preferably, suicide genes are selected according to the present invention, which allow any of the herein control mechanisms, e.g. suicide genes selected from cytosin deaminase (CD), uracil phosphoribosyl transferase (UPRTase), HSV thymidine kinase (HSV-Tk), suicide genes which may be induced by addition of tetracycline such as the bacterial Tet repressor protein (TetR), etc. As a particular example the cytosine desaminase (CD) may be used. The cytosine desaminase (CD) typically occurs in a variety of organisms and is capable of transforming 5-fluorocytosin (5-FC) into 5-fluorouracil (5-FU), which represents a common chemotherapeutic agent. 5-Fluorouracil (5-FU) is highly toxic for the organism whereas its prodrug 5-fluorocytosin (5-FC) is not toxic to cells. 5-Fluorouracil (5-FU) is subsequently phosphorylated by cellular kinases and is capable of abrogating the cells RNA synthesis. Thus, the prodrug 5-fluorocytosin (5-FC) represents an excellent tool for inducing suicide of a specific cell. Furthermore, 5-Fluoro-dUMP acts as antifolate agent and inhibits the enzyme thymidylat synthase, which catalyses methylation of dUMP to dTMP in the de novo synthesis path of desoxyribonucleotides. Thereby, inhibition of DNA synthesis in the cell may be achieved. Also preferably, the HSV-1 thymidin kinase (ATP: Thymidin-5-phosphotransferase) and its corresponding prodrug ganciclovir (GCV) may be used. The guanosin analog GCV is specifically phosphorylated and inhibits elongation of DNA synthesis and thus leads to suicide of the cell.

Transfection of the vectors or nucleic acids as defined herein, encoding a GLP-1 peptide or GLP-1 fusion peptides and optionally an additional factor, into suitable cells used for preparation of (spherical) microcapsules as defined herein, may be accomplished by any method known to a skilled person (see e.g. Maniatis et al. (2001) Molecular Cloning: A laboratory manual, Cold Spring Harbor Laboratory Press, Cold Spring Harbor, N.Y.). If vectors are transfected into suitable cells as defined herein, the vector is preferably present in the form of a plasmid DNA, which carries a GLP-1 peptide or GLP-1 fusion peptide encoding nucleic acid. The plasmid DNA is preferably a circular plasmid DNA. Suitable transfection methods include, without being limited thereto, e.g. electroporation techniques including modified electroporation techniques (e.g. nucleofection), calcium phosphate techniques, e.g. the calcium phosphate co-precipitation method, the DEAE-Dextran method, the lipofection method, e.g. the transferring-mediated lipofection method, etc. Preferably, transfection is carried out with plasmid DNA carrying a vector as defined herein using a modified electroporation technique (e.g. nucleofection).

The vector as defined herein or, alternatively, the nucleic acid, encoding a GLP-1 peptide or GLP-1 fusion peptides, or a fragment or variant thereof as defined herein, and optionally an additional factor as defined herein, may furthermore be complexed, e.g. for transfection with at least one synthetic polymer or a natural polymer, e.g. polyamino acids, or may be conjugated thereto. At least one polymer constituent may be covalently coupled to the vector as defined herein or, alternatively, the nucleic acid encoding a a GLP-1 peptide or GLP-1 fusion peptides, or a fragment or variant thereof as defined herein, and optionally an additional factor as defined herein. “Conjugated” in the meaning of the present invention is intended to mean “chemically coupled”. “Chemically coupled” is intended to mean coupled via covalent or non-covalent bonding. While covalent bonding may also be utilized, non-covalent bonding is preferred for transfection purposes. Thereby, the polymer constituent may be linked to the fusion peptide via complexation without covalent linkage, e.g. via hydrogen bonding or electrostatic, hydrophobic, etc., interaction.

The polymer used herein for coupling the vector as defined herein or, alternatively, the nucleic acid, encoding a GLP-1 peptide or GLP-1 fusion peptides, or a fragment or variant thereof as defined herein, and optionally an additional factor as defined herein, may be a physiologically acceptable polymer which includes polymers which are soluble in an aqueous solution or suspension and have no negative impact, such as side effects, to mammals upon administration of the fusion peptide in a pharmaceutically effective amount. There is no particular limitation to the physiologically acceptable polymer used according to the present invention. The polymer may be of synthetic nature or may be a naturally occurring polymer (e.g. a protein).

More generally, the synthetic polymer used with a vector as defined herein or, alternatively, the nucleic acid encoding a GLP-1 peptide or GLP-1 fusion peptides, or a fragment or variant thereof as defined herein, and optionally an additional factor as defined herein, is preferably selected from alkylene glycols, such as polyethylene glycol (PEG), polypropylene glycol (PPG), copolymers of ethylene glycol and propylene glycol, polyoxyethylated polyol, polyolefinic alcohol, polyvinylpyrrolidone, polyhydroxyalkyl methacrylamide, polyhydroxyalkyl methacrylate, such as polyhydroxyethylene methycrylate, polyacrylate, polysaccharides, poly([alpha]-hydroxy acid), polyvinyl alcohol, polyphosphazene, polyoxazoline, poly(N-acryloylmorpholine), polyvinylethyl ether, polyvinlyacetal, polylactic glycolic acid, polylactic acid, lipid polymer, chitin, hyaluronuic acid, polyurethyne, polysialic acid, cellulose triacetate, cellulose nitrate and combinations of any of the foregoing.

The present invention also provides a method for preparing the (spherical) microcapsules as used according to the present invention. These (spherical) microcapsules are preferably prepared according to two or more method steps. According to a method step 1) a core is prepared as disclosed above. According to a method step 2) the core as prepared according to method step 1) is coated by one or more surface coating layer(s). Further optional steps may comprise repetition of method step 2) for the preparation of additional surface coating layers. Preferably, a step identical to method step 2) is carried out for each of such additional surface coating layers. Further optional steps may include washing steps subsequent to preparation of the spherical microcapsule.

Typically, a core as disclosed herein is prepared according to method step 1) for preparing (spherical) microcapsules, as used according to the present invention. Such a core is composed of cross-linked polymer and GLP-1 encoding and secreting cells, which have been transfected according to a method as disclosed herein. According to method step 1), a mixture (suspension) of the soluble form of the polymer, e.g. the soluble form of an alginate (e.g. potassium or sodium alginate in physiological saline solution), and of GLP-1-peptide encoding and secreting cells is typically prepared, preferably in a concentration as defined herein for the (spherical) core, e.g. of 1×10⁵ up to 6×10⁷ cells, per ml polymer solution.

As a typical technique the homogenic cell/polymer suspension (e.g. cell/alginate suspension) may be pressed via an air injected spray nozzle, consisting of three channels, which are arranged concentrically as three concentric rings around a common centre: an inner channel, an intermediate channel and an outer channel (air ring). Preferably hollow needles are used for the inner channel having an inner diameter of 50 μm up to 2,000 μm. The intermediate channel typically has an inner diameter of 60 μm to 4,000 μm, and the outer channel (air ring) preferably has an inner diameter of 100 μm to 5,000 μm. Exclusively the inner channel and the outer channel (air ring) are used in method step 1) for preparing the core of the (spherical) microcapsule, as used according to the present invention. Thus, a spray nozzle merely consisting of two channels (an inner and an outer channel) may be used in method step 1) as well. Typically, no material flows through the intermediate channel, if an air injected spray nozzle with three channels is used. The suspension of the cell/polymer solution is typically pressed with a speed of 10 μl/min to 5 ml/min through the inner channel leading to droplets at the outlet of the channel, which tear off due to the air flow provided by the outer channel (air ring), having a speed of typically 0.5 l/min to 10 l/min. Droplets containing cells and non-cross-linked polymer solution fall down into a cross-linker containing solution (precipitation bath), which is typically positioned in a distance of about 4 cm to about 60 cm under the outlet of the air injected spray nozzle. The droplet preferably rounds during dropping down, thereby receiving a substantially spherical geometrical form. The cross-linker effects ionical cross-linking of the polymers and the core of the spherical (water insoluble) microcapsule is initially formed having a diameter as defined herein for the (spherical) core. The diameter of the core of the (spherical) microcapsule is dependent on size and geometry of the chosen channels used in method step 1). The cross-linker containing solution (precipitation bath) is preferably composed of bivalent cations, e.g. calcium or barium ions (5-100 mM) or other bivalent or multivalent cations, if alginates are used as polymers. Furthermore, the precipitation bath preferably contains a buffer substance (e.g. 1 mM-10 mM histidine) and sodium chloride (e.g. 290 mOsmol±50 mOsmol). Other suitable cross-linkers and buffers known in the art may be used herein, if other polymers than alginates are used.

Method step 1) provides the core of the (spherical) microcapsule composed of cross-linked polymers and cells as defined herein. Subsequent to method step 1) optional method step(s) may include a washing step. The core of the (spherical) microcapsule, as used according to the present invention, is e.g. washed with a physiological saline solution or any other suitable washing solution and, if applicable, the core is incubated in a sodium sulfate solution, preferably in a sodium sulfate solution according to U.S. Pat. No. 6,592,886, the disclosure of which is incorporated herein by reference. Separation of the cores of the (spherical) microcapsules, as used according to the present invention, from the precipitation bath and/or the washing bath is typically is carried out using a centrifuge or any other suitable method.

According to method step 2) the core of the (spherical) microcapsule, as used according to the present invention, prepared by method step 1) is coated with a surface coating layer substantially of cross-linked polymer. Accordingly, the core of the (spherical) microcapsule, prepared by step 1), is added to a polymer solution containing non-crosslinked polymers as disclosed herein comprising no cells. Preferably, the polymers are provided in their non-cross-linked form in a concentration as defined herein. Typically, this mixture containing the polymer solution and the core of the (spherical) microcapsule is pressed through the inner channel of the herein-described air injected spray nozzle, e.g. with a speed of 15 NI/min to 2 ml/min, preferably 10 μl/min to 5 ml/min. Simultaneously, a pure non-cross-linked polymer solution without cells, preferably a solution comprising about 0.1% to about 4% (w/v) polymer, e.g. an alginate solution without any cells, is pressed through the intermediate channel with a speed of typically 15 μl/min to 2 ml/min, preferably 10 μl/min to 5 ml/min. Thereby, droplets are formed at the end of the intermediate channel, containing the core and a surface of non-polymerized polymer. These droplets tear off due to the air flow provided via the outer channel (air ring) having a speed of typically 0.5 l/min to 10 l/min. The polymer concentration of the core of the (spherical) microcapsule, the polymer solution, into which the core of the (spherical) microcapsules is added, and the polymer concentration of the surface coating may differ (see herein). The droplets containing the core of the (spherical) microcapsules (prepared according to method step 2) fall into a solution containing the cross-linker (precipitation bath) as defined herein. During dropping down, the droplet preferably rounds to an approximately spherical geometrical form. The cross-linker affects an ionic cross-linkage of the polymers analogous to method step 1). Thereby, water insoluble (spherical) microcapsules are formed having a diameter as defined herein, preferably of total diameter (particle size) of the (spherical) microcapsule of about 100 μm to about 200 μm, more preferably a total diameter of about 115 μm to about 185 μm, even more preferably a total diameter of about 130 μm to about 170 μm, and most preferably a total diameter of about 145 μm to about 155 μm, e.g. about 150 μm. The total diameter of (spherical) microcapsules obtainable by method step 2) is dependent from size and geometry of the chosen channels, as used herein. In order to prepare (spherical) microcapsules as defined herein, with more than one surface coating layer, i.e. the (spherical) microcapsules containing the core as defined herein and 2, 3, 4, 5, 5-10 or more surface coating layers, method step 2) may be repeated as often as necessary. Those further surface coating layers are defined within the herein diameter ranges.

Subsequent to method step 2) one or more optional washing steps may follow as defined herein.

According to a further aspect the present invention also provides a method of treatment of AMI or MI in an animal, preferably a mammal. Such a method of treatment may therefore be used in the field of either human medicine or veterinary medicine. In the context of the present invention the term mammal typically comprises any animal and human, preferably selected from the group comprising, without being restricted thereto, humans and (mammalian) (non-human) animals, including e.g. pig, goat, cattle, swine, dog, cat, donkey, monkey, ape or rodents, including mouse, hamster and rabbit, cow, rabbit, sheep, lion, jaguar, leopard, rat, pig, buffalo, dog, loris, hamster, guinea pig, fallow deer, horse, cat, mouse, ocelot, serval, etc. Such a treatment typically occurs by administration of (spherical) microcapsules as defined herein to a patient in need thereof, particularly by the administration of cells as defined herein, e.g. mesenchymal stem cells or mesenchymal stromal cells, or any other cell (type), that may be used in the context of the present invention, encoding and secreting a GLP-1 peptide as defined herein, a GLP-1 fusion peptide as defined herein, or a fragment or variant thereof, wherein these cells are encapsulated in a (spherical) microcapsule as defined herein to prevent a response of the immune system of the patient to be treated. Preferably, the (spherical) microcapsule as well as all its components as used in the inventive method, e.g., polymers of the polymer matrix of the core or the surface coating, etc., is as defined above.

Treatment in the context of the present invention preferably comprises treatment or prevention of ischemic heart disease or acute coronary syndromes and the treatment or prevention of conditions associated therewith. Non-limiting examples of such diseases or conditions include AMI or MI, ST elevation MI (STEMI), cardiomyopathy (including ischemic cardiomyopathy), unstable angina, congestive heart failure and ventricular dysfunction, heart failure, endothelium dysfunction disorders, optionally hypertension, or any diseases or conditions related thereto.

A method of treatment or prevention of AMI or MI as defined herein including treatment or prevention of a disease or condition associated with AMI or MI furthermore comprises administering the cells, encapsulated in a (spherical) microcapsule as defined herein or the (spherical) microcapsule as defined herein, or administering the pharmaceutical composition containing such (spherical) microcapsule, to a patient in need thereof. A patient in need thereof is typically a, e.g., an animal, preferably a mammal, such as a human being. Administration in the context of the herein method of treatment typically occurs in a “safe and effective” amount of the active agent, i.e. the cells, encapsulated in a (spherical) microcapsule as defined herein, or the (spherical) microcapsule as defined herein. As used herein, “safe and effective amount” means an amount of these cells, encapsulated in a (spherical) microcapsule as defined herein, or the (spherical) microcapsule as defined herein, that is sufficient to significantly induce a positive modification of a disease or disorder as mentioned herein. At the same time, however, a “safe and effective amount” is small enough to avoid serious side-effects that is to say to permit a sensible relationship between advantage and risk. The determination of these limits typically lies within the scope of sensible medical judgment. In the context of the present invention the expression “safe and effective amount” preferably means an amount of the cells, encapsulated in a (spherical) microcapsule as defined herein, or the (spherical) microcapsule as defined herein that is suitable to exert beneficial effects known for GLP-1, e.g. its activity to powerfully reduce the damages caused by ischemia or oxygen shortage and potential death of heart tissue without the need of repeated administration of GLP-1 peptide(s) and/or the risk of an undesired immune response against e.g. implanted GLP-1 expressing allogenic cells. A “safe and effective amount” of the cells, encapsulated in a (spherical) microcapsule as defined herein, or the (spherical) microcapsule as defined herein, will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the administering doctor.

Typically, (spherical) microcapsules as contained in the inventive pharmaceutical composition secrete about 0.2 μg GLP-1 peptide as defined herein per day per ml of (spherical) microcapsules. Thus, a dosage range may be e.g. in the range from about 0.01 μg to 20 mg of secreted biologically active GLP-1 peptide per day (even though higher amounts in the range of 1-100 mg are also contemplated), such as in the range from about 0.01 μg to 10 mg per day, preferably in the range from 0.01 μg to 5 mg per day and even more preferably in the range from about 0.01 μg to 1 mg per day and most preferably in the range from about 0.01 μg to 500 μg per day.

Administration in the context of the herein method of treatment typically occurs by providing the cells, encapsulated in a (spherical) microcapsule as defined herein, or the (spherical) microcapsule as defined herein, or the pharmaceutical composition containing such (spherical) microcapsule, into a specific administration site in the patient to be treated. Such a specific administration site is typically the heart muscle or tissue, the myocardium or myocardial tissue, particularly the infarct area, the surrounding area and/or the periinfarct zone, e.g. all as detected by conventional means in the art such as electrocardiography or NOGA electromechanical mapping or MRI. Administration sites also include blood vessels of or around the heart, e.g. arterioles feeding the heart muscle or tissue, arterioles feeding the myocardium or myocardial tissue, particularly arterioles feeding the infarct area, the surrounding area and/or the periinfarct zone, etc., e.g. the LAD of the heart (LAD=left anterior descending (LAD) coronary artery), or other coronary arteries. Administration sites furthermore include any surface of the heart muscle or tissue, particularly of the infarct area, the surrounding area and/or the periinfarct zone, which may be treated, without being limited thereto, by epicardial injection after or during open chest surgery, where infarct may be discriminated by eye. Following infarct, the tissue rapidly becomes jelly-like and loses physical integrity. Injection of the cells, encapsulated in a (spherical) microcapsule as defined herein, or the (spherical) microcapsule as defined herein, or the pharmaceutical composition containing such (spherical) microcapsule, may occur also into this liquefied jelly-like area of the infarct or in any further region as defined herein.

If administration is carried out, e.g. by administering the (spherical) microcapsule into a blood vessel of or around the heart, such as arterioles feeding the heart muscle or tissue, arterioles feeding the myocardium or myocardial tissue, particularly arterioles feeding the infarct area, the surrounding area and/or the periinfarct zone, etc., e.g. the LAD of the heart (LAD=left anterior descending (LAD) coronary artery), or other coronary arteries, the inventive (spherical) microcapsule are typically administered in an amount and a time, which prevents occulsion and any embolic effect, such as an infarct of the heart, microinfarcts, etc. This may be achieved by e.g. administering the total amount of (spherical) microcapsules to be administered, e.g. about 5,000 to about 1,000,000 beads, about 10,000 to about 750,000 beads, about 10,000 to about 500,000 beads, about 10,000 to about 250,000 beads, or about 10,000 to about 100,000 beads, e.g. about 40,000 to about 100,000 beads, e.g. about 40,000, about 50,000, about 60,000, about 70,000, about 80,000, about 90,000 or about 100,000 beads, about 60,000 beads e.g. corresponding to e.g. about 3 to 4 million cells, or about 100,000 to about 300,000 beads, e.g. about 100,000, about 150,000, about 200,000, about 250,000 or about 300,000 beads, or any range formed by any two of these values. Administration preferably occurs in a slow speed or a time staggered mode. As an example, up to 10,000,000 beads may be slowly administered into the left anterior descending (LAD) coronary artery without causing an infarct.

Administration of the cells, encapsulated in a (spherical) microcapsule as defined herein, or the (spherical) microcapsule as defined herein, or the pharmaceutical composition containing such (spherical) microcapsule, into a specific administration site as defined herein may be carried out using different modes of administration. The cells, encapsulated in a (spherical) microcapsule as defined herein, or the (spherical) microcapsule as defined herein, or the pharmaceutical composition containing such (spherical) microcapsule, can be administered, for example, systemically or locally. Routes for systemic administration in general include, for example, transdermal or parenteral routes, including intravenous, subcutaneous, and/or intraarterial injections. Routes for local administration in general include, e.g., topical administration routes but also transdermal, intramuscular, subcutaneous, intracardial, intramyocardial and/or pericardial injections. More preferably, the cells, encapsulated in a (spherical) microcapsule as defined herein, or the (spherical) microcapsule as defined herein, or the pharmaceutical composition containing such (spherical) microcapsule, may be administered by an intradermal, subcutaneous, or intramuscular route, more preferably by an intracardial, intramyocardial and/or pericardial injection.

Other modes of administration, which may be suitable for treatment of any of the afore mentioned diseases or disorders, include transplantation of the cells as defined herein, encoding and secreting GLP-1, a fragment or variant thereof or a fusion peptide comprising GLP-1 or a fragment or variant thereof, wherein these cells, are encapsulated in a (spherical) microcapsule as defined herein, or of (spherical) microcapsules as defined herein, (preferably formulated in a suitable form, e.g. by addition of suitable pharmaceutical carriers, e.g. in the form of gels, capsules, tablets, etc.). Cells encapsulated in a (spherical) microcapsule as defined herein, may be directly delivered to the affected site of the heart (an administration site as defined herein) by interventional means, e.g. using a catheter to navigate to the affected area and implant the beads by injection into the myocardial tissue.

Implantation could be performed during routine angioplasty post AMI. Implantation may also occur into the affected area of myocardium of a mammalian AMI or post AMI patient by direct injection into the heart tissue, or by intravascular delivery through the arterioles feeding the affected heart tissue.

Without being limited thereto, the cells as defined herein, encoding and secreting GLP-1, a fragment or variant thereof or a fusion peptide comprising GLP-1 or a fragment or variant thereof, wherein these cells, are encapsulated in a (spherical) microcapsule as defined herein, or the (spherical) microcapsules as defined herein may be administered e.g. via injection by applying an appropriate injection needle such as injection needles having a size of from 12 to 26 G, more preferably of from 18 to 22 G or e.g. by transplanting the cells or the (spherical) microcapsules as defined herein, preferably formulated in a suitable form, using surgical devices, such as scalpels, injection needles as defined herein, etc. According to a particular example, which shall not be regarded as limiting to the present embodiment, a patient in need thereof, suffering from AMI or MI or any disease associated thereto or disclosed herein may receive a intramuscular injection or implantation of the cells or the (spherical) microcapsules as defined herein into a site of administration as defined herein, etc.

Treating or preventing AMI or MI diseases and disorders as defined herein using (spherical) microcapsules as defined herein having cells embedded in its core encoding and secreting GLP-1 or such cells or a pharmaceutical composition comprising these (spherical) microcapsules) preferably results from the beneficial effects of GLP-1, e.g. its activity to (powerfully) reduce the damages caused by ischemia or oxygen shortage and potential death of heart tissue. Such beneficial effects include e.g. improved regional and global LV function in patients with AMI and severe systolic dysfunction after successful primary angioplasty, etc. The in situ cardioprotective effect of (spherical) microcapsules encoding and secreting GLP-1 is particularly due to the local secretion of a glucagon-like peptide-1 (GLP-1) fusion protein and other paracrine factors that are continuously delivered to the site of injury.

According to the knowledge of the present inventors, without being bound thereto, the in situ cardioprotective effect of (spherical) microcapsules encoding and secreting GLP-1 is at least in part based on the fact that the present invention successfully utilizes properties of GLP-1 which have the potential for it to exert a direct cardioprotective effect. In this regard and in addition to its incretin actions, GLP-1 has been shown to reduce pancreatic beta-cell apoptosis. The localization of the GLP-1 receptor in the heart and the demonstration that GLP-1 promotes the activity of PI3K in beta-cells (a kinase that has been clearly associated with myocardial protection in the setting of ischemic/reperfusion injury as well as preconditioning), and thus allows one to hypothesize a novel and independent action of GLP-1 in the setting of ischemia/reperfusion. GLP-1 additionally induces an increased level of cAMP in cardiomyocytes, which, in turn, activates protein kinase A. GLP-1 has an antiapoptotic action on insulin-secreting cells mediated by cAMP and PI3K. Activation of PI3K leads to the phosphorylation and inactivation of the proapoptotic peptide BAD by causing it to bind to 14-3-3 proteins. BAD is a proapoptotic member of the Bcl-2 family that can displace Bax from binding to Bcl-2 and Bcl-xl, resulting in cell death. Western blot results of the investigators confirmed phosphorylation of BAD at serine 136 by GLP-1. Therefore, it appears as a surprising result, that GLP-1 has a direct antiapoptotic effect on cardiac muscle in model used. Furthermore, elevated levels of cAMP have previously been thought to be detrimental in ischemic cardiomyocytes. Nevertheless, the amount of cAMP produced may play a role in determining divergent signalling pathways that lead to antiapoptotic pathways. The cAMP produced may also be located in particular microdomains, described as compartmentalization, that restrict its actions. GLP-1-mediated increases in cAMP (comparable to isoproterenol) failed to cause any inotropic or lusitropic effect, supporting the suggestion for such compartmentalization. Compartmentalization of G protein-coupled signalling has been the subject of numerous reports, and it is increasingly recognized that spatiotemporal regulation of protein kinase A activity involves regulation of discrete cAMP pools. GLP-1 has also been shown to increase blood pressure and heart rate in rats, although the prior art failed to demonstrate any hemodynamic changes. This may be due to the differences in dose, method of delivery, or species. GLP-1 has been shown to have effects on the central control of blood pressure and pulse. Additionally, recombinant GLP-1 has previously been shown in a porcine model of myocardial ischemia to prevent the accumulation of pyruvate and lactate but failed to show any decrease in the infarction.

Nevertheless, the inventors of the present invention surprisingly could demonstrate myocardial protection by GLP-1 or a fusion peptide of GLP-1 as shown herein. In conclusion, the inventors of the present invention have surprisingly found for the first time a cardioprotective effect of GLP-1 in pig heart, and, furthermore, a new insight into this possible therapeutic potential for GLP-1 agonists, a class of drugs currently undergoing trials in the treatment of type 2 diabetes, wherein no immunoreactions are exhibited by administering a (spherical) microcapsule as defined herein.

The invention furthermore encompasses use of cells as defined herein, encoding and secreting GLP-1, a fragment or variant thereof or a fusion peptide comprising GLP-1 or a fragment or variant thereof, wherein these cells, are encapsulated in a (spherical) microcapsule as defined herein, for the manufacture of a product, e.g. a pharmaceutical composition or a kit, for the treatment of AMI or MI in an animal, preferably a mammal, such as a human being. The cells as used in such a treatment may be cells as defined herein, e.g. mesenchymal stem cells or mesenchymal stromal cells, or any further cell, that may be used in the context of the present invention, encoding and secreting GLP-1, a fragment or variant thereof or a fusion peptide comprising GLP-1 or a fragment or variant thereof, wherein these cells, are encapsulated in a (spherical) microcapsule to prevent a response of the immune system of the patient to be treated.

Another aspect of the present invention is a pharmaceutical composition containing cells as defined herein, encoding and secreting GLP-1, a fragment or variant thereof or a fusion peptide comprising GLP-1 or a fragment or variant thereof, wherein these cells, are encapsulated in a (spherical) microcapsule as defined herein, or containing (spherical) microcapsules as defined herein. Such a pharmaceutical composition may be applied to a patient suffering from the herein disorders, preferably to the administration sites as defined herein in a mode as defined herein.

Preparation of pharmaceutical compositions which contain cells as defined herein, encoding and secreting GLP-1, a fragment or variant thereof or a fusion peptide comprising GLP-1 or a fragment or variant thereof, wherein these cells, are encapsulated in a (spherical) microcapsule as defined herein, or containing (spherical) microcapsules as defined herein, as an “active ingredient”, is generally well understood in the art, as e.g. exemplified by U.S. Pat. Nos. 4,608,251; 4,601,903; 4,599,231; 4,599,230; 4,596,792; and 4,578,770, all incorporated herein by reference.

Typically, pharmaceutical compositions are prepared as injectables either as liquid solutions or suspensions, preferably containing water (aqueous formulation) or may be emulsified. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

For intramuscular, intravenous, cutaneous or subcutaneous injection, or any further injection at the site of affliction as defined herein, the cells or (spherical) microcapsules as defined herein will be in the form of a parenterally acceptable aqueous solution which is pyrogen-free and has suitable pH, isotonicity and stability. Liquid pharmaceutical compositions generally include a liquid vehicle such as water. Preferably, the liquid vehicle will include a physiological saline solution, dextrose ethanol or other saccharide solution or glycols such as ethylene glycol, propylene glycol or polyethylene glycol or combinations thereof may be included. Further examples include other isotonic vehicles such as physiological salt solutions, e.g. Ringers solution or Lactated Ringer's solution.

If the inventive pharmaceutical composition comprises an aqueous solution of cells or (spherical) microcapsules as defined herein, and e.g. a buffer, said (spherical) microcapsule is typically present in the pharmaceutical composition in a concentration from 0.1 mg/ml or herein, and said pharmaceutical composition usually has a pH from about 2.0 to about 10.0, preferably from about 7.0 to about 8.5.

It is possible that other ingredients may be present in the inventive pharmaceutical composition. Such additional ingredients may include wetting agents, emulsifiers, antioxidants, bulking agents, pH buffering agents (e.g. phosphate or citrate or maleate buffers), preservatives, surfactants, stabilizers, tonicity modifiers, cheating agents, metal ions, oleaginous vehicles, proteins (e.g. human serum albumin, gelatin or proteins) and/or a zwitterion (e.g. an amino acid such as betaine, taurine, arginine, glycine, lysine and histidine). Such ingredients are selected by a skilled person according to the specific requirements of the cells embedded in the core of the (spherical) microcapsule, as used according to the present invention, i.e. the ingredients are not cytotoxic and ensure viability of the cells. Furthermore, such ingredients may stabilize GLP-1 peptides already encoded and secreted by the cells embedded in the core of the (spherical) microcapsule, as used according to the present invention.

With regard to buffers these are preferably selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, hepes, bicine, tricine, malic acid, succinate, maleic acid, fumaric acid, tartaric acid, aspartic acid or mixtures thereof. Each one of these specific buffers constitutes an alternative embodiment of the invention.

The use of all of the afore-mentioned additives in pharmaceutical compositions containing cells as defined herein and/or the (spherical) microcapsule as used according to the present invention, is well-known to the skilled person, in particular with regard to concentration ranges of the same. For convenience reference is made to Remington: The Science and Practice of Pharmacy, 19^{th} edition, 1995.

Inventive pharmaceutical compositions containing cells, encoding and secreting GLP-1 as defined herein, and/or (spherical) microcapsules as defined herein, are preferably administered in a manner as defined herein for treatments in general. Such administrations are preferably compatible with the dosage formulation, and comprise preferably a safe and effective amount of the active ingredients as defined herein, i.e. such amount which is regarded as safe but therapeutically effective. The quantity of cells, encoding and secreting GLP-1 as defined herein and/or (spherical) microcapsules as defined herein, to be administered with an inventive pharmaceutical composition (or, if required, alone), depends on the subject and the disease to be treated, including, e.g., the severity of the patient's disease. Suitable dosage ranges depend on the amount of biologically active GLP-1 peptide secreted by the (spherical) microcapsules (as contained in the inventive pharmaceutical composition) during a predetermined time period and typically range in the order of one to several hundred micrograms (GLP-1) per day as defined herein.

The present invention may furthermore comprise combinations of the herein described embodiments and features if not described otherwise and is not intended to be limited to these particularly defined single embodiments.

The invention is illustrated further in the accompanying Figures. However, it is not intended to limit the scope of the invention to the content of the Figures as shown in the following.

### DESCRIPTION OF FIGURES

FIG. 1: shows a non-limiting overview over exemplary constructs a-m (see also Example 1), which may be contained in cells used for preparation of the (spherical) microcapsules, as used according to the present invention.

FIG. 2: depicts the results of transient expression of different GLP-1 constructs in hTERT-MSC and HEK293 cells and of active GLP-1 after transient transfection (see also Example 2). Only marginal active GLP-1 levels can be found in the monomeric GLP-1 constructs #103 and #317 (having just one copy of GLP-1(7-37)). An enormous gain in expression was observed in the dimeric GLP-1 construct #217 (having GLP-1(7-37) as component (I) and as component (III)) both in hTERT-MSC and in HEK293 cells.

FIG. 3: shows a Western Blot Analysis of a cell culture supernatant from GLP-1 secreting cells (see also Example 3). Lane 1: 100 ng synthetic GLP-1(7-37) dissolved in supernatant of mock transfected hTERT-MSC cells; Lane 2: supernatant of hTERT-MSC cells (clone 79TM217/13) secreting dimeric GLP-1 from construct #217; Lane 3: supernatant of AtT20 cells (clone 81-A-217/3) secreting dimeric GLP-1 from construct #217; Lane M: prestained protein marker [kDa]). The results show that peptides as defined herein containing GLP-1(7-37) and a C-terminal appendix (2 and 3 in FIG. 3) are secreted from the transfected cell lines and can be detected using an anti-GLP-1 antibody, which binds to the mid-molecular epitopes of GLP-1(7-37).

FIG. 4: describes plasma stability tests (in vitro) carried out with GLP-1 peptides as used according to the present invention. Therefore, HEK293 cells were transiently transfected with constructs (1) #103 GLP-1(7-37), (2) #317 GLP-1(7-37)-IP2-extended with 11 AA and (3) #217 GLP-1(7-37)-IP2-GLP-1(7-37). HEK293 cells are effective hosts for the gene construct (see also Example 4).

FIG. 5: describes a plasma stability kinetic (in vitro) carried out with supernatant of stably transfected hTERT-MSC cell clone 79TM217/18K5 secreting GLP-1 peptide CM1 produced by construct #217 GLP-1(7-37)-IP2-GLP-1(7-37) and synthetic GLP-1(7-37) as control. The results are obtained from three independent experiments. Active GLP-1 was measured using the GLP-1 (active) ELISA (Linco).

FIG. 6: shows a Western Blot for the peptides indicated below. The following values are given: SEQ ID NO: 1 (ID1syn) corresponds to GLP-1(7-37), 31 aa, 3.3 kD; SEQ ID NO:8 (ID8 syn, CM3) corresponds to GLP-1(7-37)-IP2, 46 aa, 5.1 kD; SEQ ID NO: 7 (ID7rec, CM2) corresponds to GLP-1(7-37)-IP2-RR-GLP2, 83 aa, 9.4 kD; SEQ ID NO: 6 (ID6syn, CM1) corresponds to GLP-1(7-37)-IP2-RR-GLP1(7-37), 79 aa, 8.7 kD (see also Example 5).

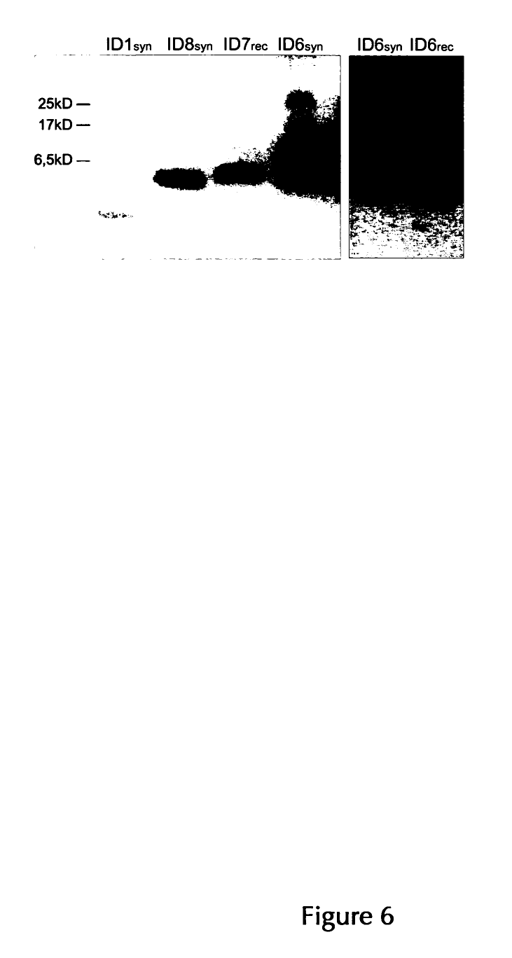

FIG. 7: illustrates dose response curves for GLP-1 receptor mediated cAMP increase in the bioassay cell line 111CHO349/18. Stimulation was done with serially diluted conditioned medium of 79TM217/18K5 cells secreting CM1 produced by construct #217 GLP-1(7-37)-IP2-GLP-1(7-37). No detectable cAMP response was found in the parental hMSC-TERT cell line. The graph was prepared from five independent experiments. The peptide dose that produces a half maximal effect (ED50) in the cAMP bioassay has been determined to be 353 μM (see also Example 6).

FIG. 8: depicts an exemplary vector used for transient and stable gene expression. The vector consists of two separate transcription units, one for the gene of interest (GOI) and one for the fusion of the suicide gene HSV thymidine kinase and the resistance gene blasticidin. For the first transcription unit, the human ubiquitin B promoter was used, and for the second transcription unit the human ferritin promoter was used (see also Example 9).

FIG. 9: illustrates characterization of cells used for (spherical) microcapsules as defined herein, after immortalising the cells in advance. As may be seen from FIG. 9A, immortalised cells are still able to differentiate into adipocytes, osteocytes and chondrocytes as their non-immortalised counterparts (left, right). Immortalised cells have fibroblastic morphology and are more homogeneous regarding size and granularity as the mortal MSCs as shown by flow cytometry e.g. using CD 44 and CD166 epitope markers which are characteristic for the primary cells used here. Immortalised cells express the same CD markers as their non immortalised counterparts (see FIG. 9B).

FIG. 10: shows the anti-apoptotic efficacy of the C-terminal elongated GLP-1 analogue CM1. Apoptosis is induced in RIN-5F cells by addition of the protein biosynthesis inhibitor cycloheximid (CHX) in a final of 10 μg/ml and 100 μg/ml respectively. The presence of different concentrations of the recombinantly in E. coli produced dimeric GLP-1 fusion protein CM1 result in an significant (p<0.01) increase of cell viability, which is quantified after an incubation period of 24 hours.

FIG. 11: is a schematic diagram of the inventive concept using (spherical) microcapsules encoding and secreting GLP-1 as utilized in the treatment of AMI and MI. Cells, e.g. mesenchymal stem cells, mesenchymal stromal cells or allogeneic cells are encapsulated in a thin selectively permeable alginate matrix forming (spherical) microcapsules encoding and secreting GLP-1. The alginate matrix is permeable for oxygen and nutrients supplying the encapsulated cells, as well as for GLP-1 or the GLP-1 fusion protein encoded and secreted by the cells. On the other hand, cells and components of the immune system cannot pass this barrier as depicted herein. Left: schematic diagram of the inventive concept using (spherical) microcapsules encoding and secreting GLP-1. The cell containing corebead (cream-coloured) is surrounded by a layer of pure alginate (grey) Right: (spherical) microcapsules encoding and secreting GLP-1 in vitro.

FIG. 12 shows a gross pathology of a heart in which coronary arteries have been embolised (black dotted lines) with alginate beads (A) or inventive (spherical) microcapsules (CellBeads) (B). The patches outlined in white on (A) represent areas of visible infarction on the surface of the heart, whereas very little infarcted area is visible in (B).

FIG. 13: shows the % of the LV infarct area on the surface of the hearts embolised with alginate control versus inventive (spherical) microcapsules (CellBeads).

FIG. 14: shows the % ejection fraction pre-embolisation, immediately post embolisation and 4 weeks post embolisation demonstrating recovery of the inventive (spherical) microcapsules (Cell Beads) group.

FIG. 15: shows a histological section of the mid LAD (LAD=left anterior descending (LAD) coronary artery)) of the pig heart after a controlled infarct of the infarct zone (IZ) territory, the border zone (BZ) territory and the remote region following intra-arterial bead delivery can be identified (from left to right)).

FIG. 16: displays a Western-Blot analysis for the GLP-1 receptor in infarct and remote tissues 4 weeks post intra-arterial bead delivery (left, overall, right: infarct and remote regions separately). The experiment ascertains a strong rationale for the use of GLP-1 in the healthy pig model. The analysis on the heart samples 4 weeks post infarction using Western blotting methods determined that cells within the pig heart possessed GLP-1 receptors. FIG. 16 shows that the receptors were indeed present (using GAPDH as loading control) and that there were no significant differences between inventive (spherical) microcapsules (CellBeads) encoding and secreting GLP-1 or alginate control, either in the infact zone or in the remote region.

FIG. 17: displays the histological section with immunochemistry for von Willibrand Factor showing the effects of inventive (spherical) microcapsules (CellBeads) encoding and secreting GLP-1 on angiogenesis in the heart. The sections of the heart from the infarct border and remote regions were stained for this purposes with von Willibrand Factor antibody staining protocol.

FIG. 18: shows the results of the angiogenic effect of inventive (spherical) microcapsules (CellBeads) encoding and secreting GLP-1 4 weeks post intra-arterial delivery (A) Overall; and (B) by region; upon staining with Willibrand Factor antibody staining protocol according to FIG. 17. Analysis of the vessels density showed that hearts with GLP-1 CellBead delivery also contained significantly more vessels, compared to control—overall (FIG. 18(A)) and by region (FIG. 18(B)). This pattern was seen within regions of the LV (apex-LV: 39.25±7.5 vs 6.8±2.2; P=0.002 and mid-LV: 34.5±3.8 vs 14.6±6.2; P=0.04). In relation to vessel size, vessels measuring 4-10 μm in diameter were most abundant, in both control and GLP-1 CellBead groups.

FIG. 19: shows histological sections demonstrating inflammatory cell infiltrate in the porcine myocardium and the staining for inflammatory cell infiltrate from the histological sections 4 weeks post intra-arterial bead delivery. The results are shown in FIG. 20.

FIG. 20: shows the results of the histological sections of FIG. 19 demonstrating inflammatory cell infiltrate in the porcine myocardium. Staining for inflammatory cell infiltrate from the histological sections 4 weeks post intra-arterial bead delivery showed statistically greater amounts of inflammatory cells for the inventive (spherical) microcapsules (CellBeads) encoding and secreting GLP-1 compared to control (FIGS. 20(A) & (B)). This suggests the paracrine effect of release of such factors such as monocyte chemotactic factor which is known to be produced by the hMSC cells, is responsible for recruiting these important cells (monocytes and neutrophils) into the region of the infarct.

FIG. 21: depicts the TUNEL staining of porcine myocardium 4 weeks post intra-arterial bead delivery. The results are shown in FIG. 22.

FIG. 22: shows the results of the TUNEL staining of porcine myocardium 4 weeks post intra-arterial bead delivery according to FIG. 21. The evaluation of apoptosis by TUNEL staining of the histological sections 4 weeks post intra-arterial bead delivery showed statistically less apoptotic cells (TUNEL +) for the inventive (spherical) microcapsules (CellBeads) encoding and secreting GLP-1 compared to control (FIGS. 22(A) & (B)).

FIG. 23: depicts exemplary microphotographs of the bright field image (A) and vitality staining (B) of 160 μm inventive (spherical) microcapsules (CellBeads) encoding and secreting GLP-1 showing exemplary inner diameters and total diameters of these inventive (spherical) microcapsules.

The invention is illustrated further in the accompanying examples. However, it is not intended to limit the scope of the invention to the content of the Examples as shown in the following.

### EXAMPLES

### Example 1

### Creation of Genetic Constructs

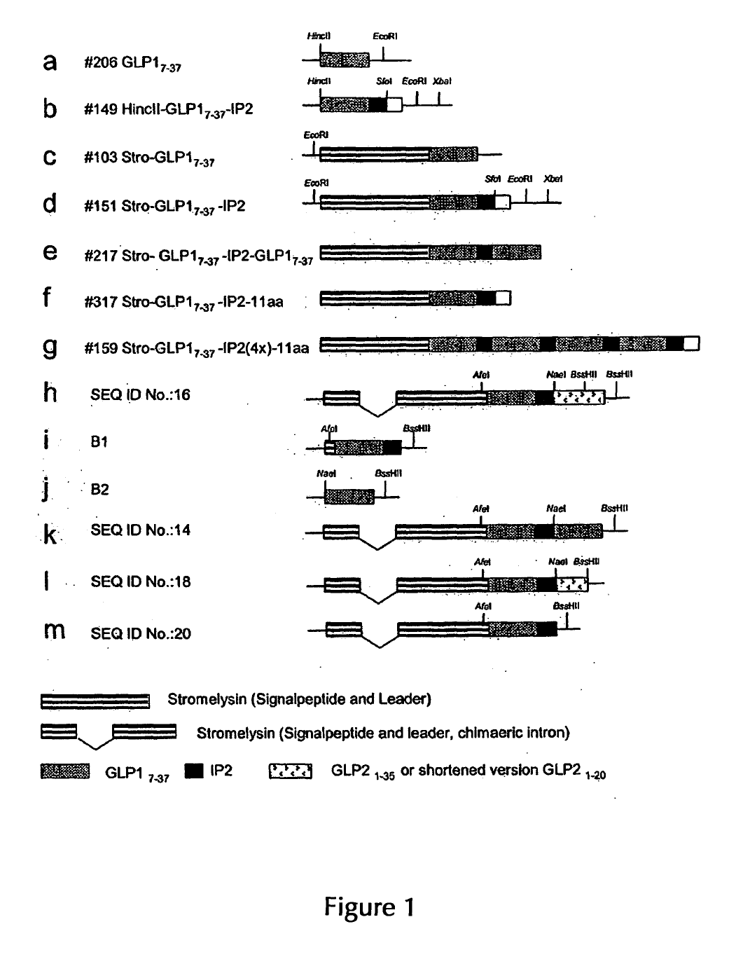

The coding sequence for GLP-1(7-37) cDNA was synthesized synthetically, in a sequence including HincII and EcoRI sites as indicated in FIG. 1a. Separately the cDNA illustrated in FIG. 1b was synthesized, including the coding sequences for GLP-1(7-37), IP2 and restriction sites for SfoO, EcoRI and XbaI, as illustrated in FIG. 1b. To direct GLP-1 to the secretory pathway, the heterologous signal sequence of stromelysin 3 (Acc. No. NM_{—}005940) was used. Therefore the cDNA, encoding stromelysin signal and leader sequence was reverse transcriptase PCR amplified from human RNA, and used with the construct of FIG. 1a or FIG. 1b to form the construct shown in FIG. 1c and FIG. 1d, respectively.

The HincII/EcoRI fragment of the FIG. 1a construct is cloned into the SfoI site of the sequence of FIG. 1d to form the construct FIG. 1e. Similarly, the EcoRI fragment of FIG. 1d is cloned into the EcoRI site of an eukaryotic expression plasmid, to produce the construct shown in FIG. 1f. To form the construct shown in FIG. 1g, the HincII/XbaI fragment of the construct shown in FIG. 1b is repetitively cloned into the SfoI/XbaI site of the construct shown in FIG. 1d. FIG. 1h shows a synthesized, codon optimized sequence encoding the stromelysin leader and signal sequences interrupted by a shortened endogenous intron sequence, fused to sequences encoding human GLP-1(7-37), IP2 and GLP-2(1-35). The DNA sequence of the construct FIG. 1h is SEQ ID NO: 16, while SEQ ID NO: 15 also shows the sequence of the translated peptide.

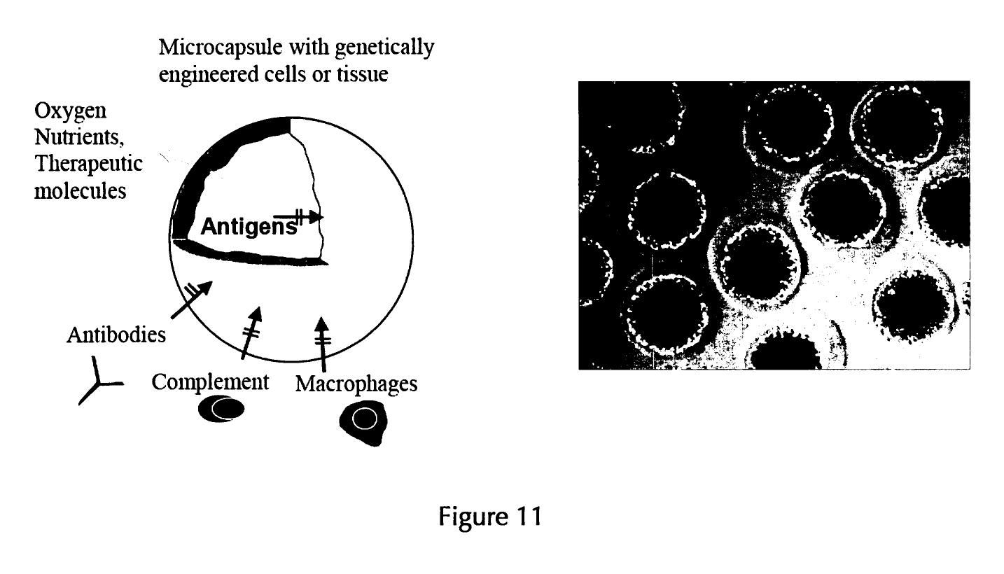

Also synthesized are the sequences in FIGS. 1i and 1j. These are then used to form the construct in FIG. 1k, by cloning the NaeI/BssHII fragment of FIG. 1j into the NaeI/BssHII linearised sequence of FIG. 1h. The DNA sequence of the construct FIG. 1k is SEQ ID NO: 14, while SEQ ID NO: 13 also shows the sequence of the translated peptide. The construct of FIG. 11 is formed by BssHII digest and religation of the sequence of FIG. 1h. The DNA sequence of the construct FIG. 11 is SEQ ID NO: 18, while SEQ ID NO: 17 also shows the sequence of the translated peptide. The construct of FIG. 1m is formed by cloning the AfeI/BssHII fragment of the sequence of FIG. 1i into the AfeI/BssHII linearised sequence of FIG. 1h. The DNA sequence of the construct FIG. 1m is SEQ ID NO: 20, while SEQ ID NO:19 also shows the sequence of the translated peptide.

The herein constructs may be made by a person skilled in the art using routine techniques.

### Example 2

### Transfection, Clonal Selection and GLP-1 Expression of Mammalian Cells

Source of the cells: HEK293 (human embryonic kidney cell line, # ACC 305, DSMZ Cell Culture Collection, Germany), AtT20 (Mouse LAF1 pituitary gland tumour cell line, #87021902, European Cell Culture Collection, UK), hTERT-MSC cells are generated and provided by Prof. Kassem, University Hospital of Odense, Denmark.

For transfection of 10⁶ cells 0.5-2 μg plasmid DNA with different GLP-1 constructs was used. The constructs were generated as described in Example 1. HEK293 cells were transfected by standard calcium phosphate co-precipitation method as described in Current Protocols in Molecular Biology (Ausubel et al. 1994ff Harvard Medical School Vol 2., Unit 9.1). AtT20 cells were transfected using FuGene (Roche) as described in Current Protocols in Molecular Biology (Ausubel et. al. 1994ff, Harvard Medical School Vol 2., Unit 9.4). Transfection of hTERT-MSC cells was performed using the Nucleofector technology (Amaxa), a non-viral method which is based on the combination of electrical parameters and cell-type specific solutions. Using the Nucleofector device (program C17) and the Nucleofector solution VPE-1001 transfection efficiencies >60% have been achieved. 48 hours after transfection selection of cell clones with stable integration of DNA into the chromosome was performed by adding the selective agent blasticidin (2 μg/ml) into the culture medium. 12-15 days later, stable transfected cell clones could be isolated and expanded for characterization.

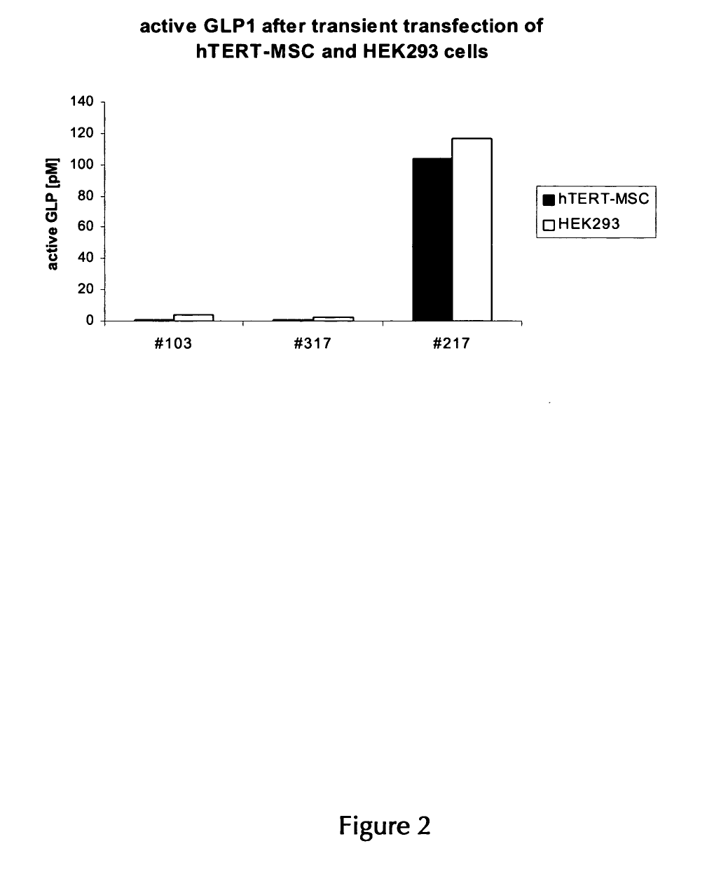

Transient expression of different GLP-1 constructs was measured in hTERT-MSC and HEK293 cells. Whereas only marginal active GLP-1 level can be found in the monomeric GLP-1 constructs #103 and #317 (having just one copy of GLP-1(7-37) an enormous gain in expression can be found in the dimeric GLP-1 construct #217 (having GLP-1(7-37) as component (I) and as component (III)) both in hTERT-MSC and in HEK293 cells. Results are summarized in FIG. 2. An elongation of the construct to the GLP-1 construct #159 (having four IP2 copies as component (II)) results in no further significant increase (not shown). After transfection of hTERT-MSC cells with different constructs clones were selected, which stably express GLP-1. The expression levels are shown in Table 1.

TABLE 1

| | | active GLP per 10⁶ cells |
|---|---|---|
| construct | cell clone | and hour [pmol] |
| #103 GLP1₍₇₋₃₇₎ | 49TM113/13 | 0.4 |
| #317 GLP1₍₇₋₃₇₎-IP2-11aa | 71TM169/1 | 0.6 |
| #217 GLP1₍₇₋₃₇₎-IP2-GLP1₍₇₋₃₇₎ | 79TM217/13 | 2.7 |

### Example 3

### Western Blot Analysis of GLP-1 Peptides, Secreted from Mammalian Cells

Cell culture supernatant from GLP-1 secreting cells was separated in a 10%-20% gradient SDS PAGE (120V, 90 minutes) and transferred to a PVDF membrane (Immobilon-P Membrane 0.45 μm Millipore IPVH 00010) by semi-dry blotting (2.0 mA/cm2, 60 minutes). After methanol fixation and blocking (3% (w:v) BSA, 0.1% (v:v) Tween-20 in TBS) the membrane was immunoblotted with 1 μg/ml anti-GLP-1 antibody (HYB 147-12, Antibodyshop) at 4° C. o/n. After washing and incubation with 0.02 μg/ml detection antibody (Anti Mouse IgG, HRP conjugated, Perkin Elmer PC 2855-1197) at RT for 4 hours, chemiluminescence detection reveals the location of the protein.

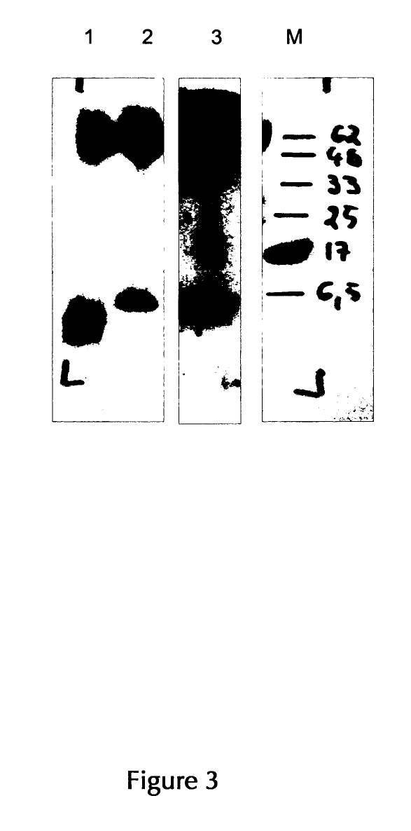

Western Blot Analysis is shown in FIG. 3 (1: 100 ng synthetic GLP-1(7-37) dissolved in supernatant of mock transfected hTERT-MSC cells, 2: supernatant of hTERT-MSC cells (clone 79TM217/13) secreting dimeric GLP-1 from construct #217, 3: supernatant of AtT20 cells (clone 81-A-217/3) secreting dimeric GLP-1 from construct #217; M: prestained protein marker [kDa]). The results show that peptides containing GLP-1(7-37) and a C-terminal appendix (2 and 3 in FIG. 3) are secreted from the transfected cell lines and can be detected using an anti-GLP-1 antibody, which binds to the mid-molecular epitopes of GLP-1(7-37).

### Example 4

### In Vitro Plasma Stability of GLP-1 Peptides Secreted from Human Cells

HEK293 and hTERT-MSC cells were transfected with constructs, encoding the heterologous stromelysin signal sequence, which is linked to GLP-1 variants encoding the following peptides:
1: GLP-1(7-37) (construct #103)
2: GLP-1(7-37)-IP2-extended with 11 AA (construct #317)
3: GLP1(7-37)-IP2-GLP1(7-37) (construct #217)

Cell culture supernatant, containing GLP-1 peptides secreted from cells or synthetic GLP-1(7-37) (Bachem) was incubated with human lymphocyte enriched plasma containing dipeptidylpeptidase activity at 37° C. and 5% CO₂, for 3 or additionally 6 and 9 hours. Synthetic GLP-1(7-37) in supernatant from mock transfected cells was used as a positive control for DPP-IV activity, which was shown to be inhibited by addition of a DPP-IV inhibitor (#DPP4, Biotrend). Active GLP was measured using the GLP-1 (Active) ELISA (#EGLP-35K, Biotrend), using an antibody which binds to the N-terminal epitope of GLP-1(7-37) discriminating the DPP-IV degraded, inactive GLP-1(9-37) peptide.

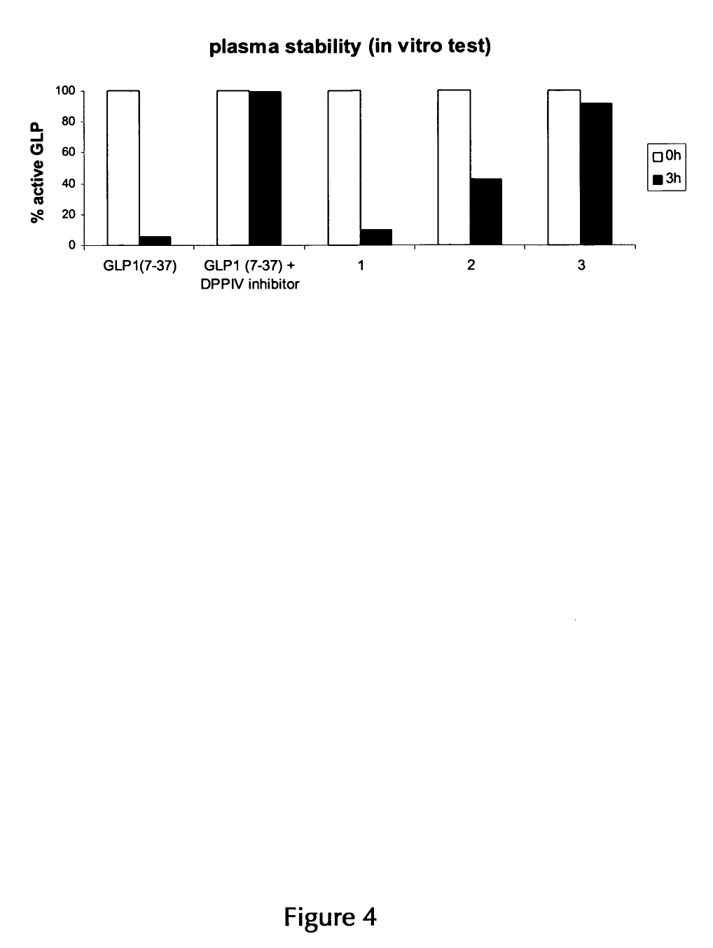

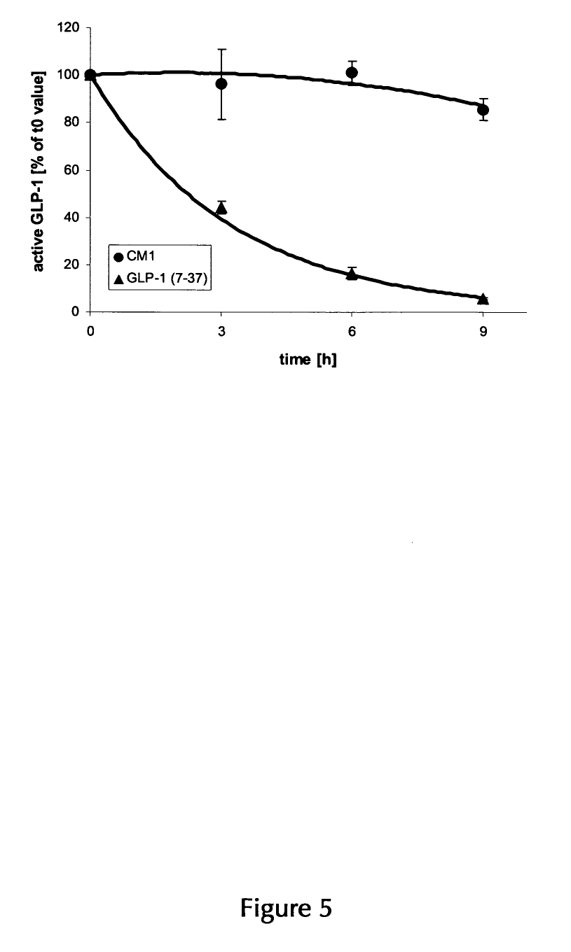

The results are shown in FIGS. 4 (HEK293 cells) and 5 (hTERT-MSC cells). HEK293 and hTERT-MSC cells are both effective hosts for the gene construct. The numbering of the results for the transfected cells is 1: supernatant of cells secreting GLP-1(7-37) from construct #103, 2: supernatant of cells secreting GLP-1 extended by IP2 and 11 aminoacids from construct #317, 3: supernatant of cells secreting dimeric GLP-1 from construct #217. While construct 1 produces wild type GLP-1 which is inactivated by DPP-IV in a similar way to synthetic GLP-1, the C-terminally elongated GLP-1 forms (2 and 3 in FIG. 4, 3 in FIG. 5) are more resistant to degradation. The C-terminal extended GLP-1 peptides are significantly stabilized in human plasma in vitro. The peptide with the dimeric GLP-1 sequence (3) is nearly fully stabilized to DPP-IV degradation in vitro.

### Example 5

### In Vitro Bioactivity of GLP-1 Peptides Measured by cAMP Release

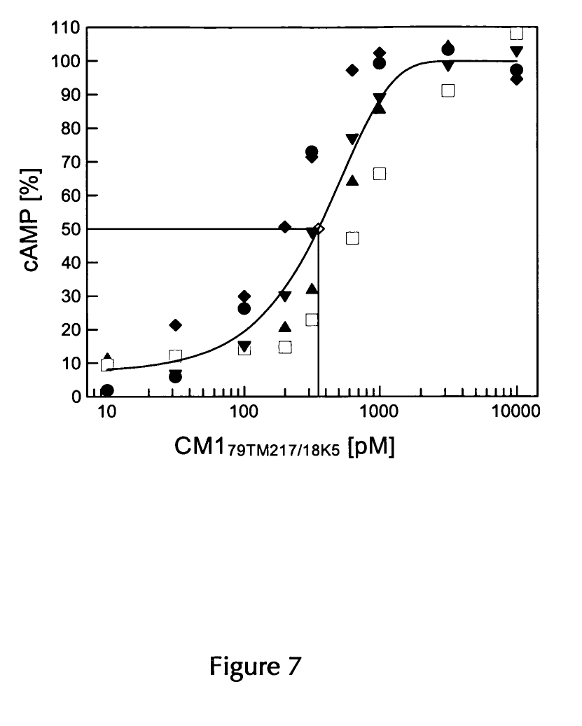

GLP-1(7-37) exerts its biological actions through the seven-transmembrane-spanning, G protein coupled GLP-1 receptor, which leads to activation of protein kinase A signalling through the second messenger cyclic AMP. To ensure that the C terminal elongation of CM1 does not interfere with GLP-1's mode of action, CM1 bioactivity was quantified in an in vitro bioassay, which determines cAMP increase in a GLP-1 receptor expressing cell line after incubation with different concentrations of the peptide. The GLP-1 receptor expressing cell line used for the study (clone 111CHO349/18) is a CHO (chinese hamster ovary) cell line stably transfected with the human GLP-1 receptor. The dose response curves for CM1 produced in the 79TM217/18K5 cells outline the bioactivity of the peptide is shown in FIG. 7. The peptide dose that produces a half maximal effect (ED50) in the cAMP bioassay has been determined to be 353 μM.

### Example 6

### In Vitro Human Plasma Stability of GLP-1^{CM} Peptides

Synthetic GLP-1 peptides (SEQ ID NO:1_{syn}, SEQ ID NO:6_{syn}, SEQ ID NO:7_{rec}, SEQ ID NO:8_{syn}) were incubated at concentrations of 20 ng/ml with human plasma at 37° C. and 5% CO, for 3 hours. Dipeptidylpeptidase activity of the plasma was inhibited by a DPP-IV inhibitor (#DPP4, Biotrend). Active GLP was measured using the GLP-1 (Active) ELISA (#EGLP-35K, Biotrend).

In contrast to the native GLP-1₍₇₋₃₇₎ (SEQ ID NO:1) the C-terminal elongated GLP-1 peptides SEQ ID NO:6, SEQ ID NO:7, and SEQ ID NO:8 are significantly stabilized in human plasma in vitro (FIG. 7). As control (on the right hand side) the results obtained for experiments with addition of DPP-IV inhibitor are shown. GLP-1 activity is completely maintained in these control experiments.

### Example 7

### Plasmid Creation

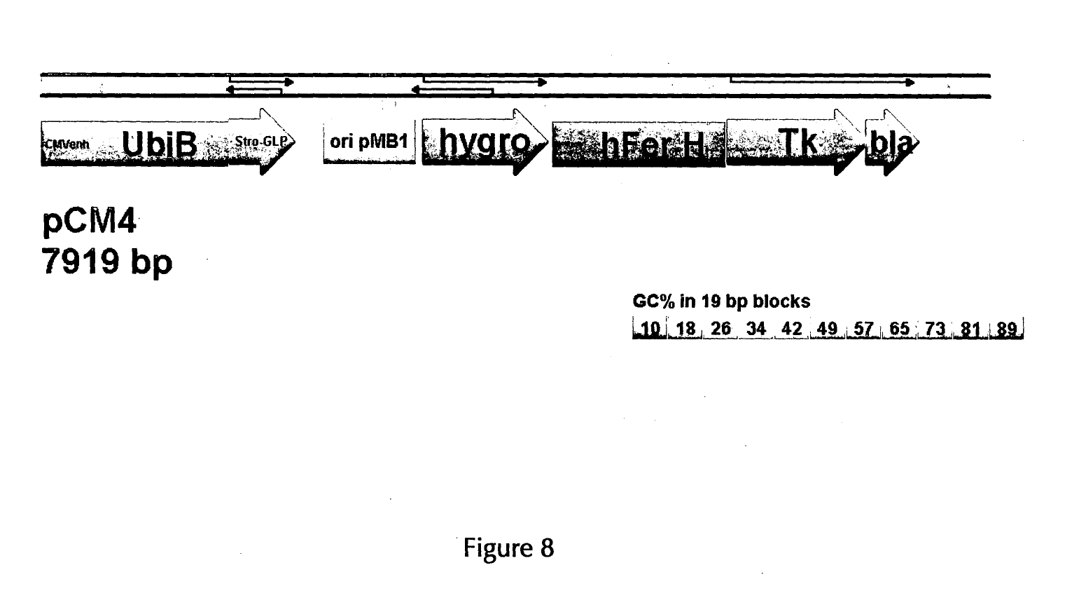

The vector for transient and stable gene expression consists of two separate transcription units, one for the gene of interest (GOI) and one for the fusion of the suicide gene HSV thymidine kinase and the resistance gene blasticidin. For the first transcription unit, the human ubiquitin B promoter was used, and for the second transcription unit the human ferritin promoter was used. The plasmid is based on plasmid pCM4, having 7,919 base pairs, shown schematically in FIG. 8.

As shown in FIG. 8, transcription unit 1, comprises the following components:
CMVenh: immediate early enhancer human cytomegalovirus
ubiB human: ubiquitin promoter B
Stro-GLP: fusion gene, coding for signal peptide and leader sequence of stromelysin and GLP1 constructs
ori pMB1: E. coli minimal origin of replication.
Hygro: hygromycin B resistance gene.

### Transcription Unit 2,

SV 40 enh: SV40 enhancer.
FerH: Human ferritin H promoter combined with 5′UTR of the murine EF1 gene.
Tk-bla: fusion gene coding for herpes simplex virus type 1 thymidine kinase and blasticidine resistance gene.

For transient expression the circular plasmid was used. For the selection of stable expressing cell clones, the plasmid was linearised and bacterial sequences (pMB1 origin and hygromycin gene) eliminated.

### Example 8

### Production of Mesenchymal Stem Cell Lines or Mesenchymal Stromal Cell Lines (MSC)

The mesenchymal stem cell line was generated by Prof. Kassem, University Hospital of Odense, Denmark (published in Simonsen et al., 2002, Nature Biotechnology 20m, 592-596) according to following criteria:

### Origin

The production cell line consists of mesenchymal stem cells (MSC), isolated from bone marrow aspirates of a healthy male donor (age 33).

### Immortalisation

Cells were immortalised by introduction of the coding sequence of the telomerase reverse transcriptase. Retroviral transduction was performed by packaging the GCsam retroviral vector in which the expression of the transgene is driven by the Moloney murine leukaemia virus long terminal repeat in PG13. Transduction was performed on day 9 (PDL 12) of culture. The cell line has so far been cultivated until population doubling level (PDL) of 260.

The insertion locus was tested by fluorescence in situ hybridization and southern blot. There is only one insertion locus of ecotopic hTERT on chromosome 5 (5q23-31). Analysis was performed at PDL 186. Giemsa banding and comparative genomic hybridization revealed that hMSC-TERT did not develop any numerical or structural chromosomal abnormalities at PDL 96 and maintained a normal diploid male karyotype. Tumourigeneity was tested in immunodeficient mice after subcutaneous implantation for six months and was found negative for PDL 80.

### Flow Cytometry (FACS) Analysis

Cells were cultured in standard growth medium to 80% confluence. Cells were trypsinised and assayed for size and granularity by FACScan flow cytometer (Becton-Dickinson). For surface marker studies typsinised cells were stained with antibodies directly conjugated to a fluorescent dye (FITC-conjugated mouse anti human CD44 monoclonal antibody, #CBL154F, Cymbus Biotechnology; phycoerythrin-conjugated mouse anti human CD166 monoclonal antibody, #559263, BD Pharmingen) for 30 min on ice. Samples were washed and fixed with 1% of paraformaldehyde until analysis with FACScan (Becton-Dickinson).

### Characterization

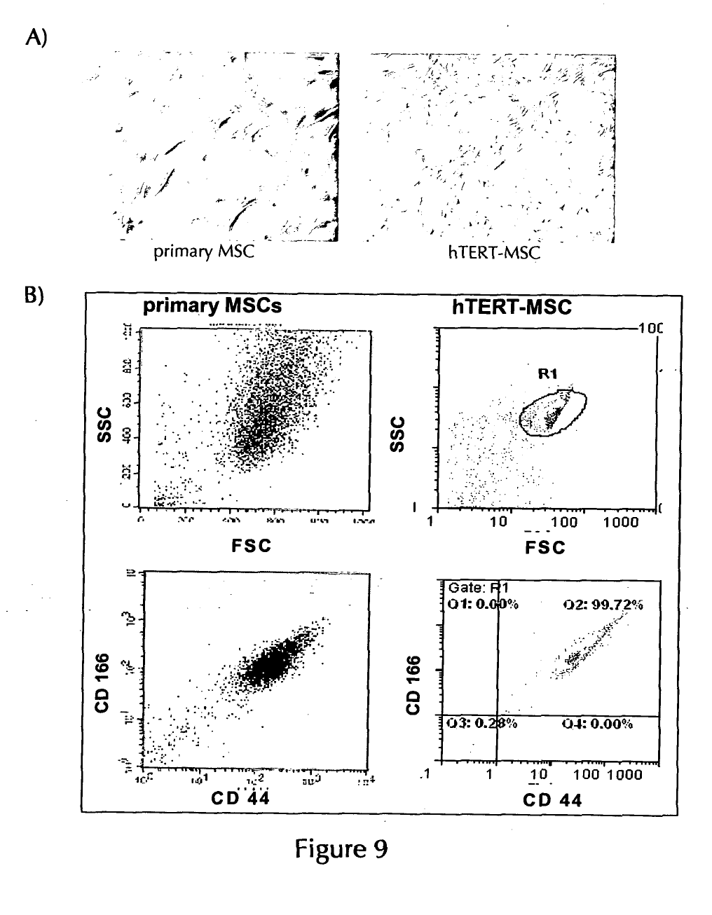

Immortalised cells are still able to differentiate into adipocytes, osteocytes and chondrocytes as their non-immortalised counterparts (see FIG. 9A). Immortalised cells have fibroblastic morphology and are more homogeneous regarding size and granularity as the mortal MSCs as shown by flow cytometry e.g. using CD 44 and CD166 epitope markers which are characteristic of the primary cells used here. Immortalised cells express the same CD markers as their non immortalised counterparts (see FIG. 9B).

### Cultivation

Serum containing medium:
7% Earles MEM
10% FCS
2 mM L-Glutamine
1 mM Sodiumpyruvate
100 U/ml Penicillin
0.1 mg/ml Streptomycin

The population doubling is between 26 and 30 hours.

### Transfection and Clonal Selection

For transefection of 10⁶ cells 0.5-2 μg plasmid DNA with different GLP1 constructs was used. HEK293 cells were transfected by standard calcium phosphate co-precipitation method. AtT20 cells were transfected using FuGene (Roche).

Transfection of hTERT-MSC cells was performed using the Nucleofector technology (amaxa), a non-viral method which is based on the combination of electrical parameters and cell-type specific solutions. Using the Nucleofector device (programme C17) and the Nucleofetor solutionVPE-1001 transfection efficiencies >60% have been achieved.

48 hours after transfection selection of cell clones with stable integration of DNA into the chromosome was performed by adding the selective agent blasticidin (2 μg/ml) into the culture medium. 12-15 days later, stable transfected cell clones could be isolated and expanded for characterization.

### Expression

Transient expression of different GLP constructs was measured in hTERT-MSC and HEK293 cells. An active GLP1 level can be found in the monomeric GLP1 constructs #103 (Stro-GLP1₍₇₋₃₇₎) and #317 (Stro-GLP1₍₇₋₃₇₎-IP2-extended with 11 aa) and an enormous gain in expression can be found in the dimeric GLP1 construct #217 (Stro-GLP1₍₇₋₃₇₎-IP2-GLP1₍₇₋₃₇₎) both in hTERT-MSC and in HEK293 cells. An elongation of construct #317 to the tetrameric GLP1 construct #159 (Stro-GLP1₍₇₋₃₇₎-IP2 (4x)-11aa) results in an similar activity (see also herein FIG. 2). After transfection of hTERT-MSC cells with different constructs clones were selected, which stably express GLP1 (see herein FIGS. 4 and 5, Example 4).

### Example 9

### Encapsulation

The cultivated cells to be encapsulated were washed with PBS (PAA, Austria) and separated using trypsin/EDTA (PAA, Austria). The reaction was quickly stopped using medium (dependent on cell type, for example RPMI, PAA, Austria) and the cell suspension centrifuged off (8 min at 1,200 rpm) The pellet was resuspended in PBS and the cell count determined. The desired quantity of 4×10⁷ cells was centrifuged off again (8 min at 1,200 rpm). The PBS was then completely removed by suction and 50 μl pellet was resuspended without air bubbles in 50 μl 0.9% saline buffered by 5 mM l-histidine to a pH of 7.4. This cell suspension was taken up in 900 μl of 1.5-1.7% (w/v) sodium alginate solution (an alginate with a viscosity of approximately 5 mPa·s of 0.2% (w/v) aqueous solution at room temperature was used).

To mix the resuspended cells with the alginate solution, the solution was drawn up in a 1 ml syringe with cannulas and homogeneously mixed with the cells by way of repeated slow drawing up and drawing off. A cell concentration of 4×10⁷ cells/ml resulted.

For producing the microcapsules with a diameter of about 200 μm, a cannula with an internal diameter of 120 μm was used in an air-charged spray nozzle. An air ring with an opening of 2.0 mm was screwed over the inner cannula. The device is an adapted version of the device described in WO 00/09566. The homogeneous cell/alginate solution mixture was dripped through the described spray nozzle. For this purpose, the 1 ml syringe containing the mixture was placed on the cannula by means of a luer connector. The cell/alginate solution mixture was pressed through the cannula at a speed of 50 μl/min. The airflow was conveyed though the outer air ring at a speed of 2.5 l/min. The resulting microcapsules precipitated into a barium-containing precipitation bath (20 mM BaCl, 5 mM L-histidine, 124 mM NaCl, pH 7.0±0.1, 290 mOsmol±3) which was constructed approximately 10 cm below the spray nozzle. After a dwell time of 5 min in the barium-containing precipitation bath the microcapsules were washed five times with 20 ml PBS in each case.

500 μl of the single-layer microcapsules were then taken up in 500 μl of a 1.5-1.7% (w/v) alginate solution the same as used for the core, herein and homogeneously mixed. This suspension was taken up in a 1 ml syringe and connected by means of a luer connector to the inner channel (internal diameter: 200 μm) of the spray nozzle and pressed at a speed of 50 μl/min therethrough. A 5 ml syringe with a 1.5-1.7% alginate solution was connected by means of a luer connector to the second inner channel (internal diameter: 700 μm) and pressed there through at a speed of 250 μl/min. The airflow was conveyed through the outer air ring at a speed of 2.9 l/min. The resultant microcapsules precipitated into a barium-containing precipitation bath (20 mM BaCl, 5 mM L-histidine, 124 mM NaCl, pH 7.0 | 0.1, 290 mOsmol±3) which is constructed approximately 10 cm below the spray nozzle. After a dwell time of 5 min in the barium-containing precipitation bath, the microcapsules were washed four times with 20 ml PBS in each case and once with medium. Two-layer microcapsules with a total diameter of approximately 180-200 μm (including the alginate layer) were produced by this process, wherein the diameter of the inner, cell containing core is 120-150 μm.

The concentration of cell in the core is about 4×10⁷ cell/ml alginate. This results in (spherical) microcapsules (CellBeads) with a bead volume of 0.002-0.004 μl containing approximately 100 cells per bead. A (spherical) microcapsule encoding and secreting GLP-1 produces on average 0.2 fmol active GLP-1 per hour.

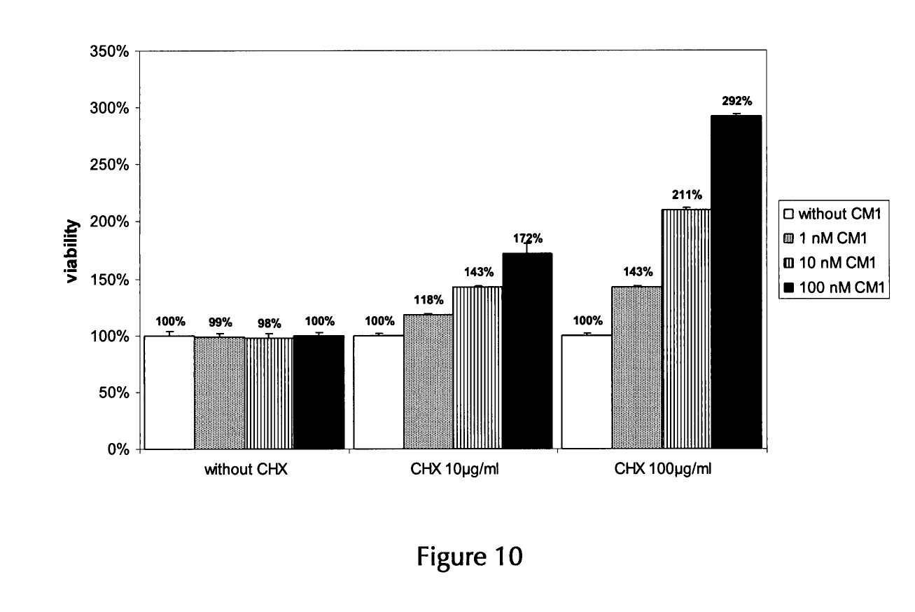

A micrograph of Cellbeads containing encapsulated GLP-1 secreting hTERT-MSC cells in the core are shown in FIG. 10.

### Example 10

### Anti-Apoptotic Efficacy of C-Terminally Elongated GLP-1

The cytoprotecitve efficacy of the C-terminally elongated GLP-1 analougue CM1 was tested in vitro using the rat insulinoma cell line Rin-5F. 40.000 Rin-5F cells were seeded per 96 well and cultivated for 2 days in RPMI supplemented with 1% L-Glutamin and 10% fetal calf serum. Apoptosis is induced after change to serumfree conditions (RPMI supplemented with 1% L-Glutamin) by addition of the protein biosynthesis inhibitor cycloheximid (CHX) in the presence of different concentrations of the recombinantly in E. coli produced dimeric GLP-1 fusion protein CM1. After 24 hours cell viability is quantified using AlamarBlue. A significant anti-apoptotic effect (p<0.01) was observed already in the presence of 1 nM GLP-1 analouge CM1. The results are given in FIG. 10.

### Example 11

### Cytokine Profile of the GLP-1 Producing hTERT-MSC Cell Line

To investigate GLP-1 independent, cytoprotective effects, the GLP-1 secreting cell line 79TM217/18K5 cell line was examined for the secretion of cytokines, chemokines and growth factors.

The cell line originates from a human stromal cell and therefore secretes a characteristic cytokine profile. A multiplex assay kit (Biosource Cytokine 30-plex) was used for measuring the 30 most abundant human cytokines, chemokines and growth factors simultaneously. No expression was found regarding the cytokines IL-1RA, IL-1β, IL-2, IL-2R, IL-4, IL-5, IL 7, IL-10, IL-12(p40/p70), IL-13, IL-15, IL-17, IP-10, EGF, Eotaxin, FGF-basic, IFN-α, IFNγ, GM CSF, G-CSF, HGF, MIG, MIP-b, MIP-1α, RANTES and TNFα (detection limit of each analyte 20 pg per 10⁵ cells and 24 h). The cytokines, which are expressed at detctable levels are summarized in table 1.

TABLE 1
Expression level of growth factors Vascular endothelial growth
factor (VEGF), neurotrophin-3 (NT-3), glial cell line-derived
neurotrophic factor (GDNF) and the cytokines Interleukin 6
(IL-6), Interleukin 8 (IL-8) and Monocyte chemotactic protein
1 (MCP-1). The factors have been quantified in cell culture
supernatant of the CM1 secreting cell line 79TM217/18K5 using
the VEGF ELISA (#ELH-VEGF-001; RayBio), NT-3 ELISA (#TB243,
Promega), GDNF ELISA (#7B221, Promega) and the the human
IL-6, IL-8 and MCP-1 ELISA Kits (RayBio).

| | Cytokine |
|---|---|
| | [pg/10⁶ cells and hour] |
| Growth factor | 79TM217/18K5 |
| VEGF | 973.0 ± 78.3 |
| NT-3 | 20.9 ± 3.5 |
| GDNF | 10.7 ± 1.5 |
| IL-6 | 378.4 ± 4.0 |
| IL-8 | 3608.7 ± 53.8 |
| MCP-1 | 16.8 ± 0.1 |

### Example 12

### Preparation of Cell Beads for a Novel Cell-Based Therapy for Myocardial Ischemia and preservation

Cells encoding and secreting GLP-1 as defined herein and (spherical) microbeads containing these cells (GLP-1 CellBeads®) for implantation into the myocardium of patients affected by acute myocardial infarction are being developed according to good manufacturing practice (GMP) according to the experiments as shown herein in previous Examples 1 to 11. The intention of this experiment is to limit damage of heart tissue by extended local delivery of GLP-1 to the myocardium. GLP-1 CellBeads® may be directly delivered to the affected site of the heart by interventional means, using a catheter to navigate to the affected area and implant the beads by injection into the myocardial tissue. Implantation could be performed during routine angioplasty post AMI. The in situ cardioprotective effect of GLP-1 CellBeads® is due to the local secretion of a glucagon-like peptide-1 (GLP-1) fusion protein and other paracrine factors that are continuously delivered to the site of injury.

The GLP-1 CellBeads® as used for this experiment consist of cells from a human mesenchymal stromal cell line, embedded in a spherical shaped alginate matrix (180-200 μm in diameter). The cells are designed to secrete GLP-1 fusion protein which seem to have an antiapoptotic effect. The alginate matrix, which entraps the cells, is generated during the herein described production process by cross-linking of the alginate with barium ions. The alginate itself has no pharmacological effect but provides a mechanical scaffold for the cells and protects them against attacks of the patient's immune system (see FIG. 11). Therefore, the alginate is regarded as an excipient. From the patient's view, the alginate matrix is considered as a safety component, because it restricts the cells locally to the point of application and prevents a free floating of the cells. To fulfil these functions properly, GLP-1 CellBeads® are composed of a corebead, which is an alginate matrix enclosing the GLP-1 fusion protein secreting cells. This corebead again is surrounded by a shell consisting of pure alginate to assure the complete encapsulation of all cells.

The cells, which are encapsulated within the alginate and secrete the GLP-1 fusion protein are derived from the clone 79TM217/18K5 and defined as the drug substance. The cells are human mesenchymal stromal cells (hMSC), which are genetically modified to produce the anti apoptotic glucagon-like peptide-1 (GLP-1) fusion protein as defined herein. The parental cell line is a non-tumourigenic cell line with normal karyotype. The cell line 79TM217/18K5 was generated at CellMed AG by genetically modifying the parental cell line. Therefore, a transfection with a plasmid vector encoding the GLP-1 fusion protein was performed.

The GLP-1 fusion protein is a dimeric GLP-1 construct, which is arranged in analogy to the native preproglucagon gene. It is a 79 amino acid dimeric GLP-1/Intervening Peptide 2 (IP-2)/GLP-1 protein with a molecular weight of 8.7 kDa and corresponds to SEQ ID No 10 in the accompanied sequence listing.

The advantages of this C-terminally elongated GLP-1 fusion protein in comparison to the native GLP-1 are the higher expression levels and the decreased susceptibility to naturally occurring degradation by dipeptidyl peptidase IV. Bioactivity of the fusion protein is maintained. For safety reasons, the plasmid used allows co-expression of the GLP-1 fusion protein and a suicide gene. The suicide gene codes for the most widely used Herpes simplex virus Type 1 Thymidine Kinase (HSV1tk). This enzyme converts intracellularly the non-toxic prodrug Ganciclovir into a toxic product, therefore allowing the destruction of transfected cells in the case of unexpected cell proliferation. Thus, systemic application of Ganciclovir to a patient treated with GLP-1 CellBeads® containing degenerated cells leads to destruction of the transplanted cells. The characterization of the cell line 79TM217/18K5 was performed taking into account the recommendations of the ICH Q5B as set forth in:
“Analysis of the Expression Construct in Cells Used for Production of r-DNA Derived Protein Products”; and
“Q5D Derivations and Characterisation of Cell Substrates Used for Production of Biotechnological/Biological Products”; and the
“Note for Guidance on the Quality, Preclinical and Clinical Aspects of Gene Transfer Medicinal Products” (EMEA/273974/2005).

### Example 13

### Proof of Concept Study

### Early Evaluation of Genetically Engineered Mesenchymal Stromal Stem Cell Therapy for Prevention of LV Dysfunction in Pigs

Background: Heart Failure (HF) is still associated with a poor prognosis with almost 50% of patients dying within five years of diagnosis. Glucagon-like Peptide-1 (GLP-1) is a naturally occurring gut incretin hormone that stimulates insulin secretion and has anti-apoptotic properties. Infusion of GLP-1 after successful primary angioplasty has previously been shown to reduce left ventricular systolic dysfunction (LVSD). We hypothesised that a prolonged delivery of GLP-1 expressing cells at the time of an MI would improve LV function and significantly reduce infarct size in a porcine model of early LV dysfunction.

Previous work has shown that bead embolisation of the coronary arteries creates a reproducible infarct. In order to evaluate the effects of locally delivered GLP-1 on the size and nature of the infarct, GLP-1 secreting CellBeads® were compared to control (non-GLP-1 secreting beads of the same size and number).

Methods: Human mesenchymal stromal cells immortalised and engineered to produce GLP-1 as defined herein in Experiments 1-12 were encapsulated into alginate beads in order to immuno-isolate the cells (GLP-1 CellBeade). These beads were selectively delivered to branches of left anterior descending coronary artery in Yorkshire White pigs (n=6), with the control group receiving cell-free alginate beads (n=6). Four weeks after intervention, hearts were explanted for morphometric quantification of infarcted surface area and histological analysis.

Results: Acute animal loss was 2 in treatment and 1 in control group. In the surviving animals, transthoracic echocardiography confirmed onset of mild LVSD (EF 40-45%) in both groups. LV surface area morphometry showed significantly decreased infarction area in the treatment group, compared to control group (21.8±4.8% vs 4.7±2.1%; p<0.018). Histological analysis showed similar patterns of fibrosis and moderately enhanced inflammation in the treatment group. Further work is aimed at investigating the effect of GLP-1 CellBeads® on calcium regulatory proteins, apoptosis and angiogenesis.

Conclusions: Delivery of alginate encapsulated mesenchymal stromal cells expressing GLP-1 (GLP-1 CellBeads®) as described according to the present invention significantly reduces infarct size and LVSD in a porcine model of early LV dysfunction.

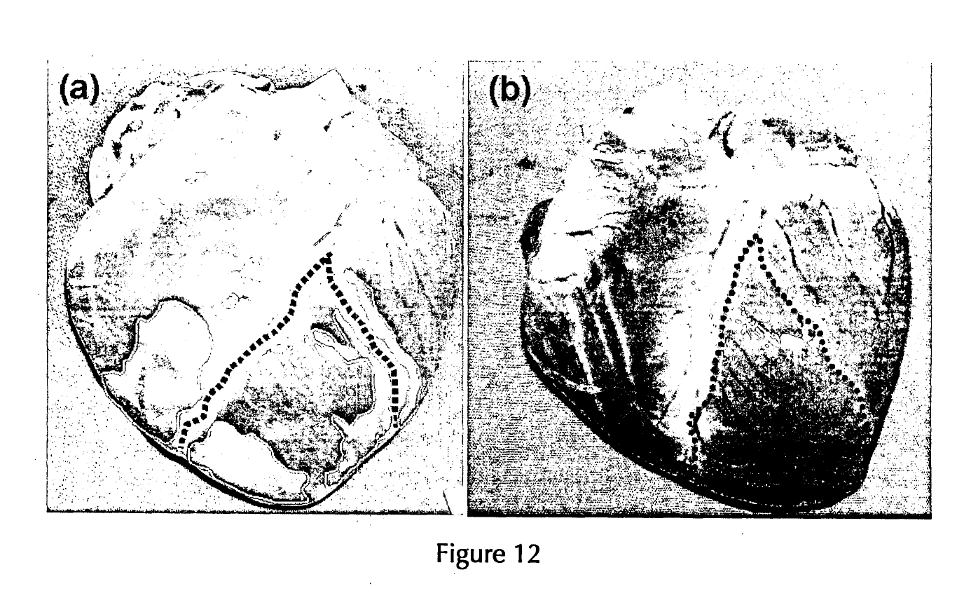

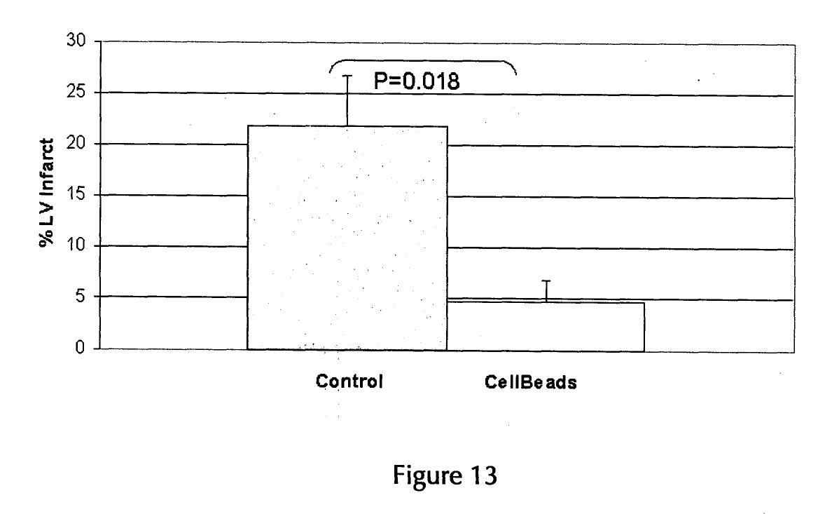

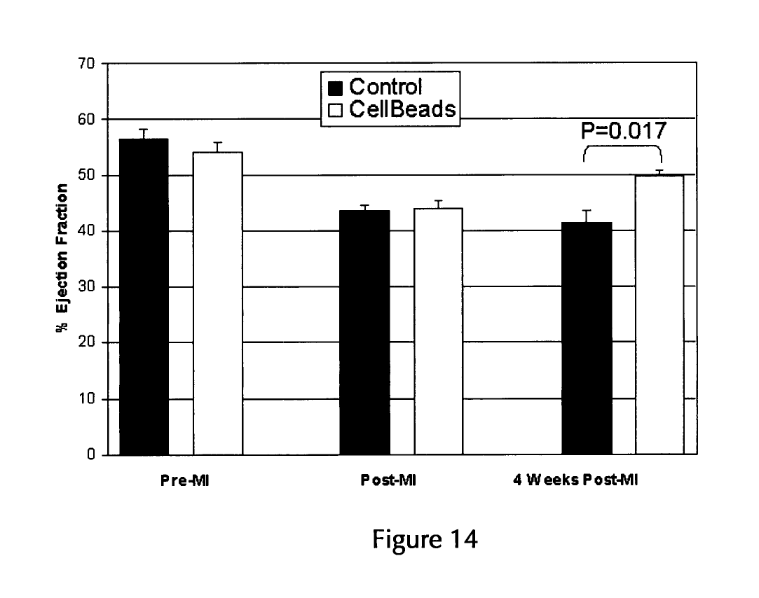

The results of Example 13 are shown in FIGS. 12, 13 and 14. FIG. 12 shows a gross pathology of a heart in which coronary arteries have been embolised (black dotted lines) with alginate beads (A) or inventive (spherical) microcapsules (CellBeads) (B). The patches outlined in white on (A) represent areas of visible infarcted area on the surface of the heart, whereas very little infarcted area in visible in (B). FIG. 13 shows the % of the LV infarct area on the surface of the hearts embolised with alginate control versus inventive (spherical) microcapsules (CellBeads). FIG. 14 shows the % ejection fraction pre-embolisation, immediately post embolisation and 4 weeks post embolisation demonstrating recovery of the inventive (spherical) microcapsules (CellBeads) group.

### Example 14

### Intra-Arterial Model of Left Ventricular Dysfunction

### Demonstration of GLP-1 Receptor Presence

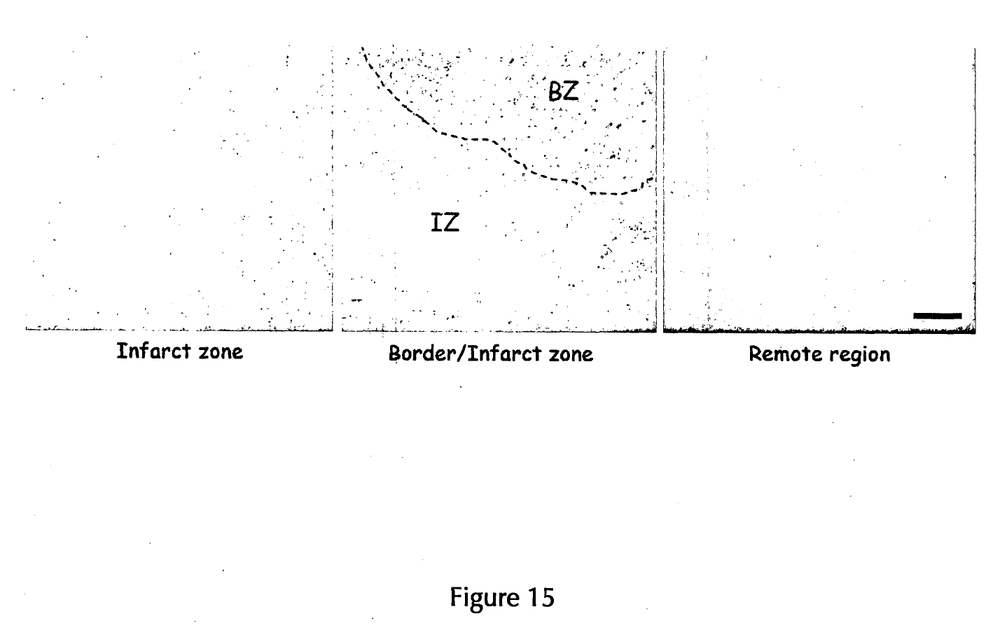

A controlled infarct was produced in pig heart by selective cathetisation into the mid LAD of the pig heart (LAD=left anterior descending (LAD) coronary artery). Histologically, three distinct zones of the myocardium were present within the sections obtained from different regions of the heart (see FIG. 15): infarct zone (IZ), border zone (BZ) and remote region (RR). Eosin staining was reduced in the IZ (FIG. 15Ci), there was a sharp demarcation between the border zone BZ and IZ (FIG. 15Cii); and the eosin staining in the BZ and remote region RR was similar (FIG. 15Ciii).

In order to ascertain a strong rationale for the use of GLP-1 in the healthy pig model, inventive (spherical) microcapsules (CellBeads) encoding and secreting GLP-1 (CellBeads) as described above, having a total diameter of 600 μm were prepared. For this purpose, mesenchymal stem cells (MSCs) were obtained from the bone marrow of a single healthy male donor aged 33 years following informed consent. Primary cells were immortalised following stable transduction by a retroviral vector containing hTERT (Simonsen et al 2002). A plasmid expression vector encoding GLP-1 was transfected into the parental cell line to produce cells that secrete a GLP-1 fusion protein which comprises of two GLP-1 molecules bound by an intervening peptide. Approximately 3,300 MSCs were embedded into each spherically shaped aliginate matrix of 600 μm consisting of the core comprised of alginate matrix enclosing the GLP-1 secreting cells and a selectively permeable surface coating layer consisting of pure alginate surrounding the core. GLP-1 CellBeads were stored in DMSO at minus 80 degrees prior to use. Following washing in Ringers solution to remove DMSO, CellBeads were suspended into Ringer's solution and further diluted immediately prior to intracoronary infusion.

For the experiments, these CellBeads were delivered to branches of left anterior descending (LAD) coronary artery in pigs, with the control group receiving cell-free alginate beads of the same size. At four weeks, hearts were explanted for morphometric quantification of infarcted surface area and analysis of apoptosis, angiogenesis and inflammation.

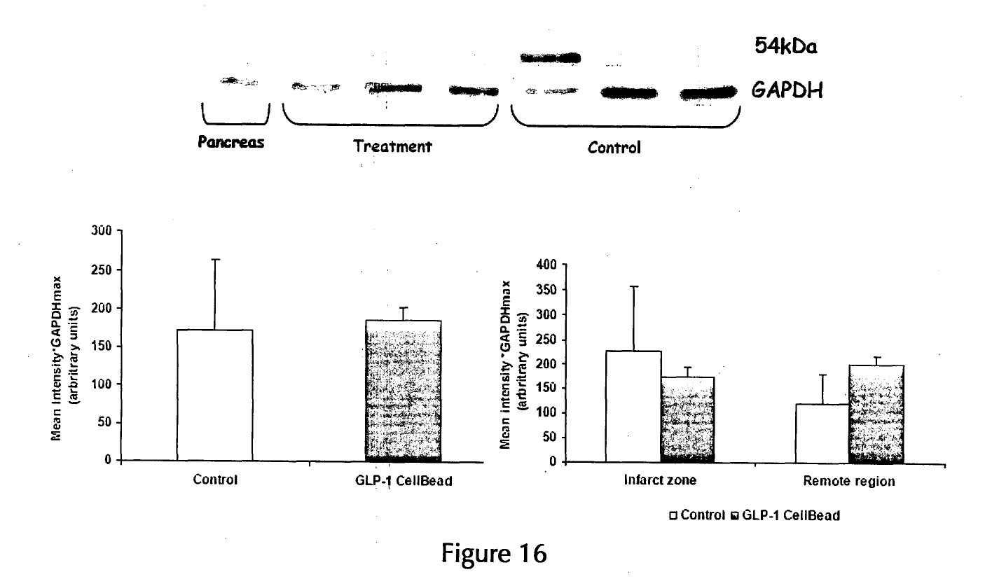

An analysis was undertaken on the heart samples 4 weeks post infarction using Western blotting methods to determine if cells within the pig heart possessed GLP-1 receptors. FIG. 16 shows that the receptors were indeed present (using GAPDH as loading control) and that there were no significant differences between CellBead or alginate control, either in the infact zone or in the remote region. Furthermore, transthoracic echocardiography confirmed onset of mild LVSD in both groups. Four weeks after intervention, repeat echocardiography demonstrated normal LV function in the treatment (CellBead) but not the control group (EF: 49.7±1 vs 41.2±2.2, p<0.01). LV surface area morphometry showed significantly decreased infarction area in the treatment (CellBead) group, compared to control group (4.7±2.1% vs 21.8±4.8%; p<0.018). Histological analysis showed enhanced inflammation in the treatment group, a significant reduction in TUNEL positive cells in the infarct and border zone of the GLP-1 CellBead group compared to the control group and significant increases in the mean number of vessels/mm² in the infarct and border zone. As a conclusion, delivery of alginate encapsulated mesenchymal stromal cells expressing GLP-1 (GLP-1CellBeads®) reduces infarct size and improves LV function in a porcine model of early LV dysfunction. This is associated with reduced apoptosis, increased angiogenesis and increased inflammatory cells.

### Example 15

### Intra-Arterial Model of Left Ventricular Dysfunction

### Demonstration of the Paracrine Effect of Inventive (Spherical) Microcapsules (CellBeads) Encoding and Secreting GLP-1 on the Heart

### Apoptosis:

Apoptosis was detected using an ApopTag kit (Millipore, UK) to detect TUNEL positive cells as per manufacturer's instructions. Immunohistochemical staining of endothelial cells surrounding capillaries was performed using an antibody against vWF antigen (Abcam, UK) and staining of granulocytes and monocytes was performed using a MAC387 anti-human Myeloid/Histiocyte antigen antibody (Dako, Denmark). Antigen retrieval sites were exposed and Vectastain ABC complex (Vector, UK) used for amplification of the signal. The sections were incubated with DAB substrate according to manufacturer's instructions. Finally, the slides were washed in distilled water and Carazzi's haematoxylin used as a counter-stain.

For analysis, photomicrographs were taken of five random fields of view within each area of the myocardium: infarct zone, border zone (lying immediately adjacent to the infarct zone) and remote region using a Leica DLMB light microscope equipped with a Leica DC300F camera. The number of TUNEL, Mac 387 and vWF antigen positive cells were counted as a proportion of the total cell number (TUNEL) or area of myocardium (Mac387 and neovessels) using Image) analysis software (NIH) with the observer blinded to the experimental conditions.

All data are expressed as mean (±Standard Error of the Mean). Students t-test was used to test for differences between treatment groups. For determination of apoptosis, angiogenesis and inflammation, data was obtained for specific regions of the myocardium, termed infarct zone border zone or remote regions. ANOVA was performed for multiple comparisons. Values of p<0.05 were considered statistically significant.

There was a significant difference in the number of TUNEL positive cells between the two groups (Control, 14.33±1.41 vs. Treatment, 7.65±0.40, p=0.0063). In terms of individual areas of the myocardium, significantly more TUNEL positive cells were observed in the IZ and BZ of the control group compared to the GLP-1 CellBead group (Control IZ, 42.46±4.27 vs. Treatment IZ, 22.81±1.24, p=0.0072; Control BZ, 0.40±0.11 vs. Treatment BZ, 0.10±0.03, p=0.02). No significant difference in the number of TUNEL positive cells in the RR existed between the two groups (Control RR, 0.12±0.05 vs. Treatment RR, 0.06±0.02, p=NS). For each treatment group, the mean number of TUNEL positive cells varied significantly between area (p<0.0001 for both treatment groups). Using Bonferroni's multiple comparisons test for the control group, the difference between the mean number of TUNEL positive cells in the IZ and the BZ was significant in both groups (p<0.05 for both) but the difference between the mean number TUNEL positive cells in the BZ and the RR was not significant in both groups.

### Angiogenesis:

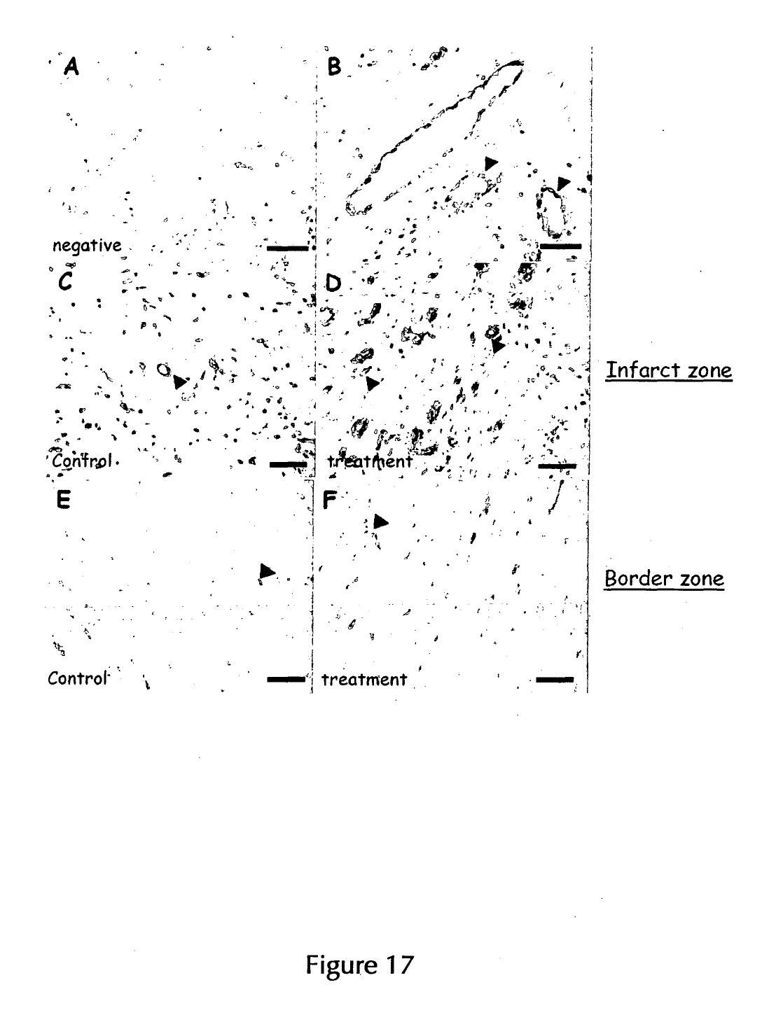

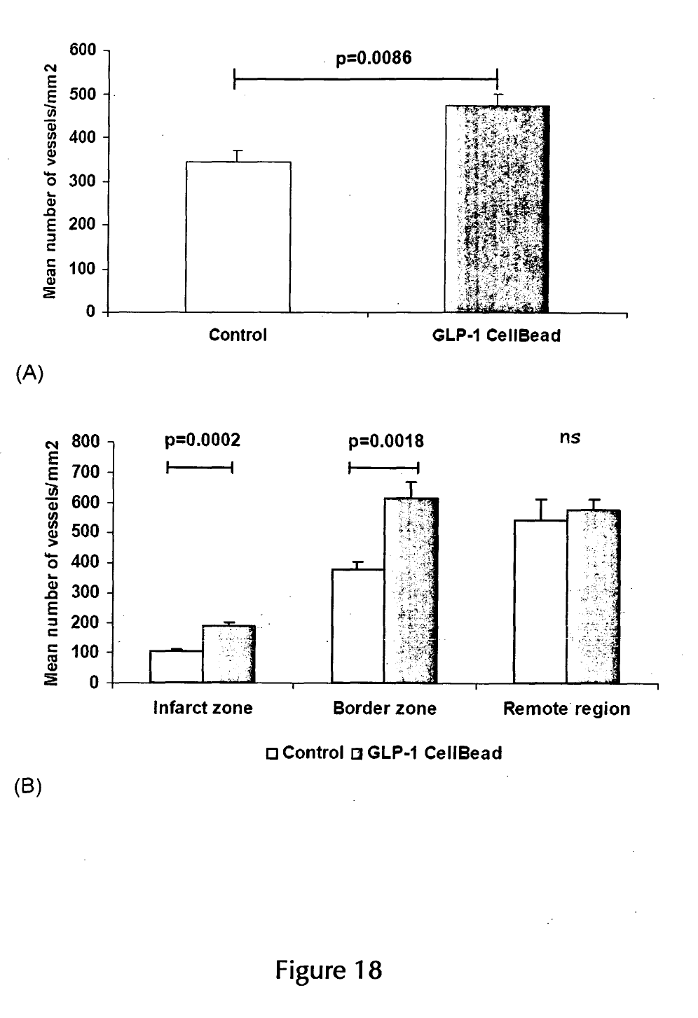

Sections of heart from the infarct, border and remote regions were stained with a standard von Willibrand Factor antibody staining protocol (see FIG. 17). Analysis of the vessels density showed that hearts with GLP-1 CellBead delivery (inventive (spherical) microcapsules (CellBeads) encoding and secreting GLP-1) also contained significantly more vessels, compared to control—overall (FIG. 18(A)) and by region (FIG. 18(B)). This pattern was seen within regions of the LV (apex-LV: 39.25±7.5 vs 6.8±2.2; P=0.002 and mid-LV: 34.5±3.8 vs 14.6±6.2; P=0.04). In relation to vessel size, vessels measuring 4-10 μm in diameter were most abundant, in both control and GLP-1 CellBead groups.

### Localised Inflammatory Cell Numbers:

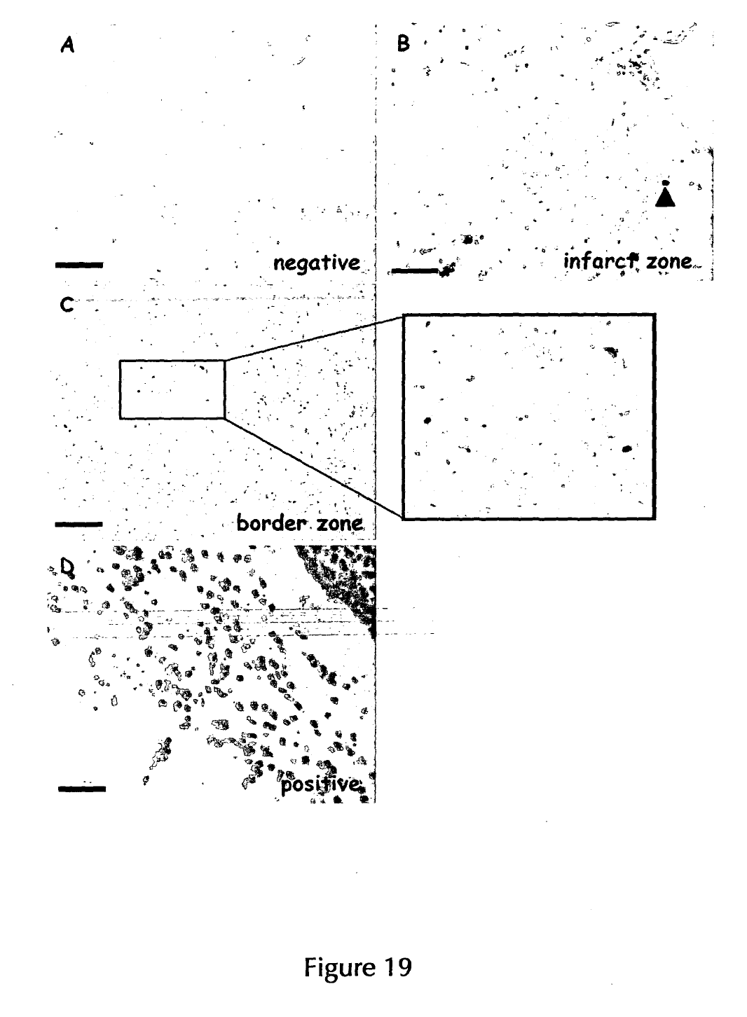

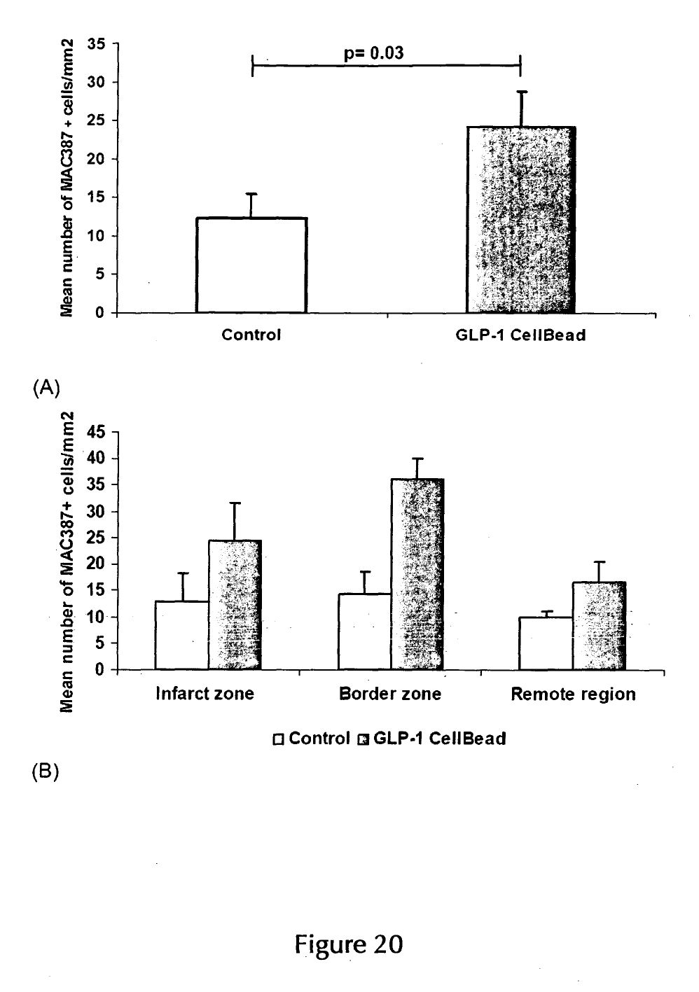

Staining for inflammatory cell infiltrate from the histological sections (FIG. 19) 4 weeks post intra-arterial bead delivery showed statistically greater amounts of inflammatory cells for the inventive (spherical) microcapsules (CellBeads) encoding and secreting (GLP-1) CellBeads) compared to control (FIGS. 20(A) & (B)). This suggests the paracrine effect of release of such factors such as monocyte chemotactic factor which is known to be produced by the hMSC cells, is responsible for recruiting these important cells (monocytes and neutrophils) into the region of the infarct.

### Anti-Apoptosis:

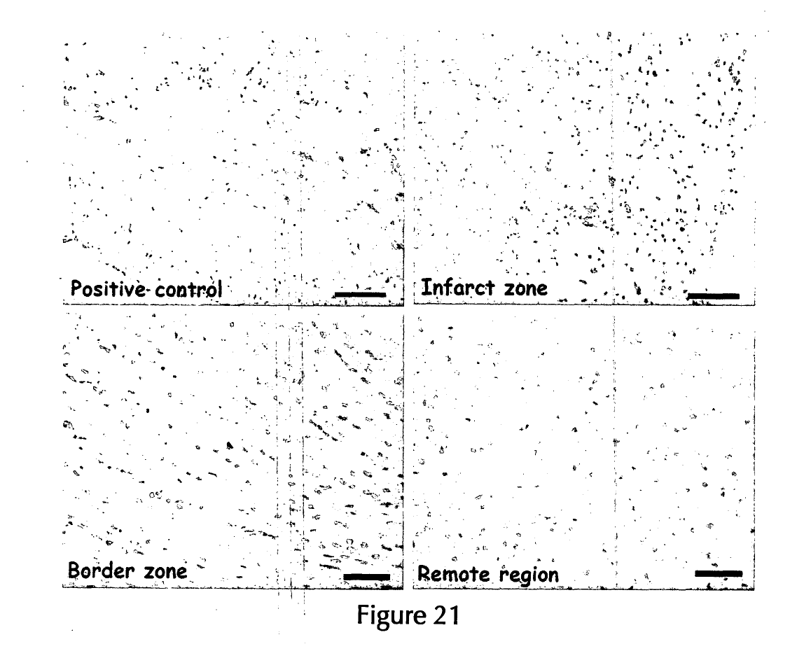

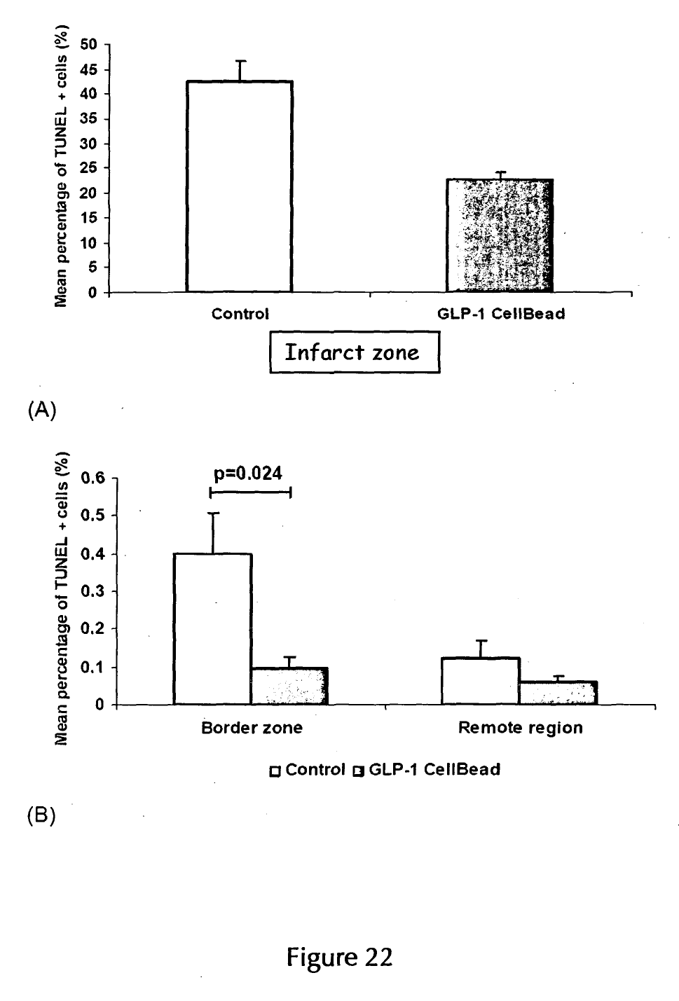

Evaluation of apoptosis by TUNEL staining of the histological sections (FIG. 21) 4 weeks post intra-arterial bead delivery showed statistically less apoptotic cells (TUNEL +) for the GLP-1 CellBeads compared to control (FIGS. 22(A) & (B)).

### Example 16

### Preparation of 160 μm CellBeads for Intra-Coronary Infusion

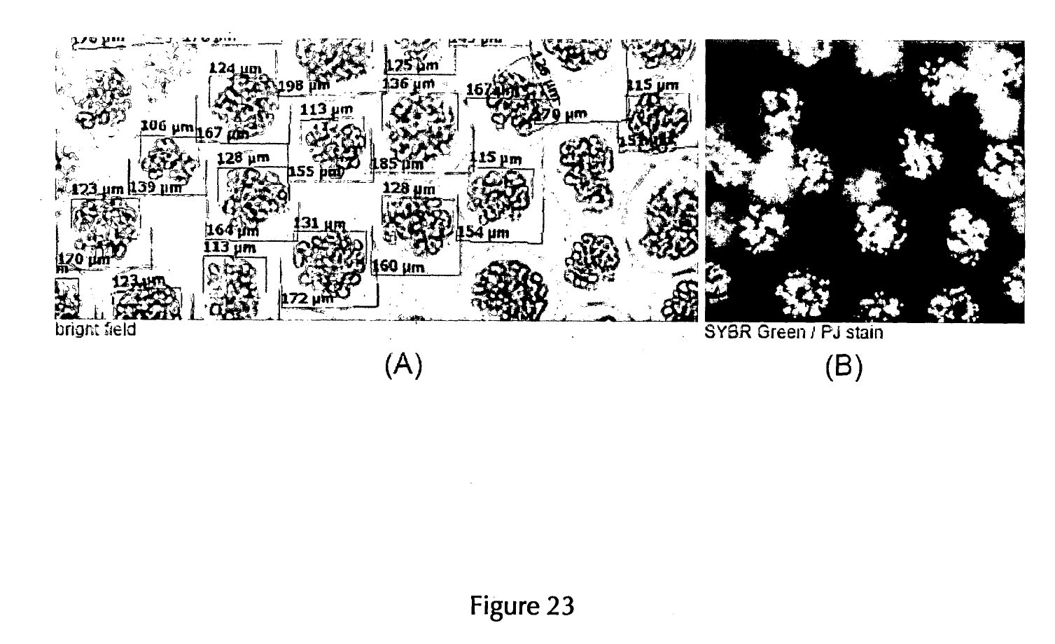

Inventive (spherical) microcapsules (CellBeads) of 160 μm total diameter encoding and secreting GLP-1 were prepared as outlined in previous Examples 1-12 for intra-coronary infusion described in Example 17. The specification of the CellBeads made for this evaluation is shown in Table 2. Microphotographs show the bright field image of the CellBeads (FIG. 23(A)) and vitality staining (FIG. 23(B)).

TABLE 2

| Test | Result |
|---|---|
| Sterility | Sterile |
| Endotoxin content | <0.05 I.U./ml |
| Mycoplasma | No mycoplasma |
| Mean total diameter of CellBeads (n = 30) | 160 ± 18 μm |
| Mean diameter of core (n = 30) | 119 ± 13 μm |
| Number of CellBeads per 100 μl CellBeads | 29 800 |
| Vitality of encapsulated cells | 82% |
| Number of cells per CellBead | 63 |
| Cell count per 100 μl CellBeads | 1.9 × 10⁶ |
| GLP-1 expression per 100 μl CellBeads | 3 pmol/h |

### Example 17

### Intra-Coronary Delivery of 160 μm CellBeads into the Coronary Arteries of Healthy Porcine Heart

### Methodology:

Four animals were treated with 500 μl of inventive (spherical) microcapsules (CellBeads) encoding and secreting GLP-1 (CellBeads) with a total diameter of 160 μm suspended in 250 mL of Ringer's Lactate (approximately 600 CellBeads/ml). A Twin-Pass intracoronary infusion catheter was positioned in segment 7 of the LAD (directly after the first diagonal branch) such that beads were delivered in the mid LAD. A Volcano Combowire was used as guide wire and kept in position during the experiment in order to measure coronary flow distal of the infusion catheter. CellBeads were administered by syringe pump at a rate of 4 mL/min in Animal 1 and 2 and 2 ml/min in Animal 3 and 4, in 50 ml aliquots. As CellBeads tend to sediment rapidly, the Cell Bead suspension was rocked frequently. At periodic intervals of 25 or 50 ml, the administration was stopped and coronary flow reserve (CFR) measured by administering adenosine intravenously. Adenosine causes maximal coronary vasodilatation and the ratio between the average peak velocity (APV) of blood flow pre and post infusion depicts the CFR. Coronary blood flow was also measured at these time points by coronary angiography and an estimate was made from the angiogram using the conventional TIMI score (TIMI-3 normal flow; TIMI-2 sluggish flow; TIMI-1 slow flow; TIMI-0 no flow). During the experiment the electrocardiogram was monitored continuously for signs of ischemia and arrhythmias.

### Summary of Results:

All three experiments according to Experiments 14, 15 and 16 were a procedural success and it proved to be possible to infuse CellBeads selectively via an intra coronary route (see Table 3). In the first animal 125 mL of CellBead suspension (50%) could be administered without signs of ischemia and reduction of flow (TIMI 3; CFR 1.4 (Baseline 1.5)). However, between 140 and 145 mL (58% of total) several premature ventricular complexes were seen that eventually graduated into ventricular fibrillation.

The full dose of inventive (spherical) microcapsules (CellBeads) encoding and secreting GLP-1 (CellBeads) could be administered in animals 2 & 3 without occurrence of significant arrhythmic events. Animal 2 suffered some ST elevation and decrease in APV and CFR. TIMI-flow was 0 in the distal LAD after infusion of 200 mL, but TIMI 3 flow remained in the diagonal branch. After administration of 250 mL, there was a 30 minute wait period and TIMI-flow was assessed again. Surprisingly, there was some recovery of flow from TIMI 0 to TIMI 1 and the APV rose slightly, which might be indicative of redistribution of the beads more distal in the arterial bed.

In animal 3, inventive (spherical) microcapsules (CellBeads) encoding and secreting GLP-1 (CellBeads) were administered at a lower infusion rate of 2 ml/min to determine if slower infusion would result in more redistribution and thus to a larger absolute amount of CellBeads to be delivered before the occurrence of coronary flow reduction. In this animal, there was no ST-elevation, APV and CFR finished at higher values and TIMI flow was graded as 3 with slight slowing of flow only after 250 mL of CellBead infusion. Animal 4 received the similar treatment as animal 3. However, in this animal a reduction of flow to TIMI 1 was seen after infusion of 70% of the beads (175 mL of CellBeads suspension) together with ischemic ECG changes. After 200 mL of CellBeads suspension blood flow was further reduced to TIMI 0, which remained also after infusion of the remainder of CellBeads.

Blood pressure remained stable in all four animals showing no significant acute effect of CellBead infusion on cardiac performance. At necropsy, the hearts were sectioned and the area of delivery isolated for histological sampling. The anteroseptal wall of the heart (perfusion territory of the LAD) felt physically stiffer in all animals suggesting some oedema/stunning from ischemia or infarction by the beads. Pathohistologic examination of H&E stained slides of the target area showed the presence of CellBeads in several small arteries (100-150 micron), as opposed to arteries in the remote area. Mononucleated cells that did not have the appearance of mononuclear peripheral blood cells were clearly visible in the center of some CellBeads, underscoring the feasibility of the current approach.

TABLE 3

| Rate | | | Volume Administered | | | | | | | | |
|---|---|---|---|---|---|---|---|---|---|---|---|
| (mL/min) | Measure | Baseline | 50 | 100 | 125 | 150 | 175 | 200 | 225 | 250 | Comments |
| Animal 1 | APV | 23 | 11 | 28 | 21 | † | † | † | † | † | |
| 4 | CFR | 1.5 | 1.8 | UNK | 1.4 | † | † | † | † | † | |
| | TIMI-flow | 3 | 3 | 3 | 3 | † | † | † | † | † | |
| | ST | X | X | X | PVCs | | | | | | PVCs and |
| | Elevation | | | | | | | | | | VF at 145 ml |
| | | | | | | | | | | | infused |
| Animal 2 | APV | 22 | 17 | 13 | 6-7 | 8 | — | 9 | — | 9 | |
| 4 | CFR | 1.4 | 1.4 | 1.2 | 1.3 | — | — | 1.3 | — | 1.1 | |
| | TIMI-flow | 3 | 3 | 1 | 2 | 1 | — | 0 | — | 0 | Possible |
| | | | | | | | | | | | thrombus |
| | | | | | | | | | | | noted on |
| | | | | | | | | | | | wire |
| | ST | X | X | | | | | | | | STE at |
| | Elevation | | | | | | | | | | ~75 mL |
| Animal 3 | APV | 17 | 29 | 18 | 16 | 16 | 13 | 13 | 17 | 13 | Wire |
| | | | | | | | | | | | repositioned |
| | | | | | | | | | | | after |
| | | | | | | | | | | | 50 mL & 200 mL |
| 2 | CFR | 3.1 | 1.2 | — | — | 1.1 | — | 1.6 | — | — | No final |
| | | | | | | | | | | | CFR as |
| | | | | | | | | | | | wire |
| | | | | | | | | | | | moved |
| | TIMI-flow | 3 | 3 | 3 | 3 | 3 | 3 | 3 | 3 | 3 | Slightly |
| | | | | | | | | | | | slowing |
| | | | | | | | | | | | flow at |
| | | | | | | | | | | | 250 mL |
| | ST | X | X | X | X | X | X | X | X | X | Normal |
| | Elevation | | | | | | | | | | throughout |
| Animal 4 | APV | 20 | 17 | 12 | 11 | 11 | ? | — | — | — | |
| 2 | CFR | 1.8 | 1.3 | 1.6 | — | 1.5 | — | — | — | — | |
| | TIMI-flow | 3 | 3 | 3 | 3 | 3 | 1 | 0 | 0 | 0 | Decrease |
| | | | | | | | | | | | of flow |
| | | | | | | | | | | | after 175 mL |
| | ST | X | X | X | X | X | | | | | Ischemia at |
| | Elevation | | | | | | | | | | 175 mL |

In the current experiments, we have advantageously utilised MSCs that have been genetically modified to express a fusion protein of GLP-1. Furthermore, the cells have been encapsulated into an alginate matrix which offers immunoprotection whilst allowing diffusion of molecules, including therapeutic agents, out of the coating. In addition, the alginate coating may assist in preventing adverse reactions to transplanted MSCs such as myocardial tumour formation (Wolfe et al 2009) and areas of calcification and ossification (Breitbach et al 2007) as recently described. Finally, we also demonstrated improved EF in injured myocardium following delivery of GLP-1 vs control CellBeads.

As a main finding of the above experiments delivery of alginate encapsulated mesenchymal stromal cells expressing GLP-1 (GLP-1 CellBeads®) according to the present invention reduces infarct size and improves LV function in a porcine model of early LV dysfunction. These findings are associated with reduced apoptosis, increased angiogenesis and increased inflammatory cell infiltrate. The findings suggest that alginate encapsulated mesenchymal stromal cells expressing GLP-1 (GLP-1CellBeade) according to the present invention represents a useful treatment for damaged myocardium and the treatment of AMI, MI and disorders related thereto.

1. A method for the treatment of acute myocardial infarction (AMI or MI) comprising administering to a subject in need thereof cells, encoding and secreting GLP-1, a fragment or variant thereof or a fusion peptide comprising GLP-1 or a fragment or variant thereof, wherein the cells, encoding and secreting GLP-1, a fragment or variant thereof or a fusion peptide comprising GLP-1 or a fragment or variant thereof are encapsulated in a spherical microcapsule to prevent a response of the immune system of the patient to be treated.

2. The method according to claim 1, wherein the spherical microcapsule preferably comprises a spherical core and at least one surface coating layer,
wherein the spherical core comprises or consists of a mixture of cross-linked polymers and cells, encoding and secreting GLP-1, a fragment or variant thereof or a fusion peptide comprising GLP-1 or a fragment or variant thereof; and
wherein the at least one surface coating layer comprises or consists of cross-linked polymers.

3. The method according to claim 1, wherein the spherical microcapsule has a total diameter of about 120 μm to about 800 μm, a total diameter of about 120 μm to about 700 μm, a total diameter of about 150 μm to about 650 μm, or a total diameter of about 165 μm to about 600 μm, or even a total diameter of about 120 μm to about 300 μm, a total diameter of about 150 μm to about 250 μm, a total diameter of about 165 μm to about 225 μm; or a total diameter of about 180 μm to about 200 μm, including a total diameter of about 180, 185, 190 or 200 μm.

4. The method according to claim 1, wherein the cells are mesenchymal stem cells, mesenchymal stromal cells, human mesenchymal stern cells, differentiated cells derived from human mesenchymal stem cells, allogenic cells or autologous cells encoding and secreting GLP-1, a fragment or variant thereof or a fusion peptide comprising GLP-1 or a fragment or variant thereof.

5. The method according to claim 2, wherein the cross-linked polymer is selected from the group comprising biopolymers and alginates.

6. The method according to claim 5, wherein the cross-linked polymer of the core and/or the at least one surface coating layer comprises a chemically identical polymer in identical or differing concentrations, wherein the polymers further may have different molecular weights and/or may be cross-linked differently.

7. The method according to claim 1, wherein the spherical microcapsule comprises 1, 2, 3, 4, 5, 5-10 or more surface coating layers.

8. The method according to claim 1, wherein the spherical microcapsule comprises an additional external surface coating layer consisting of polycations.

9. The method according to claim 1, wherein the GLP-1 is a peptide selected from the group consisting of:
a) a peptide comprising aa 7-35 of GLP-1; or
b) a peptide comprising aa 7-36 of GLP-1 or GLP-1(7-36)amide; or
c) a peptide comprising aa 7-37 of GLP-1; or
d) a peptide comprising the sequence according to formula II:
(SEQ ID NO: 59)
Xaa7-Xaa8-Glu-Gly-Thr-Phe-Thr-Ser-Asp-Xaa16-Ser-
Xaa18-Xaa19-Xaa20-Glu-Xaa22-Xaa23-Ala-Xaa25-
Xaa26-Xaa27-Phe-Ile-Xaa30-Trp-Leu-Xaa33-Xaa34-
Xaa35-Xaa36-Xaa37,
wherein Xaa7 is L-histidine; Xaa8 is Ala, Gly, Val, Leu, Ile or Lys; Xaa16 is Val or Leu; Xaa18 is Ser, Lys or Arg; Xaa19 is Tyr or Gln; Xaa20 is Leu or Met; Xaa22 is Gly or Gln; Xaa23 is Gln, Glu, Lys or Arg; Xaa25 is Ala or Val; Xaa26 is Lys, Gln or Arg; Xaa27 is Glu or Len; Xaa30 is Ala, Glu or Arg; Xaa33 is Val or Lys; Xaa34 is Lys, Gln, Asn or Arg; Xaa35 is Gly; Xaa36 is Arg, Gly or Lys or amide or absent; Xaa37 is Gly, Ala, Glu, Pro, Lys, amide or is absent; or
e) a peptide comprising the sequence according to formula III:
(SEQ ID NO: 60)
Xaa7-Xaa8-Glu-Gly-Thr-Phe-Thr-Ser-Asp-Val-Ser-
Xaa18-Tyr-Leu-Glu-Xaa22-Xaa23-Ala-Ala-Xaa26-Glu-
Phe-Ile-Xaa30-Trp-Leu-Val-Xaa34-Xaa35-Xaa36-
Xaa37,
wherein Xaa7 is L-histidine; Xaa8 is Ala, Gly, Val, Len, Ile or Lys; Xaa 18 is Ser, Lys or Arg; Xaa22 is Gly or Gln; Xaa23 is Gln, Glu, Lys or Arg; Xaa26 is Lys, Glu or Arg; Xaa30 is Ala, Glu or Arg; Xaa34 is Lys, Gln or Arg; Xaa35 is Gly; Xaa36 is Arg or Lys, amide or is absent; Xaa37 is Gly, Ala, Gln or Lys, amide or is absent, or
f) a peptide showing an identity of at least 80% with any of the herein peptides according to a) to e).

10. The method according to claim 1, wherein the GLP-1 fusion peptide or a fragment or variant thereof comprises components (I) and (II), wherein component (I) N-terminally is selected from the group of peptides consisting of or comprising the sequence of
a) a GILD-1(7-35, 7-36 or 7-37) sequence, or
b) a sequence according to SEQ ID NO: 1; or
c) a peptide comprising or consisting of the sequence according to formula II:
(SEQ ID NO: 59)
Xaa7-Xaa8-Glu-Gly-Thr-Phe-Thr-Ser-Asp-Xaa16-Ser-
Xaa18-Xaa19-Xaa20-Glu-Xaa22-Xaa23-Ala-Xaa25-
Xaa26-Xaa27-Phe-Ile-Xaa30-Trp-Leu-Xaa33-Xaa34-
Xaa35-Xaa36-Xaa37,
wherein Xaa7 is L-histidine; Xaa8 is Ala, Gly, Val, Lea, Ile or Lys; Xaa16 is Val or Len; Xaa18 is Ser, Lys or Arg; Xaa19 is Tyr or Gln; Xaa20 is Len or Met; Xaa22 is Gly or (Mu; Xaa23 is Gln, Gln, Lys or Arg; Xaa25 is Ala or Val; Xaa26 is Lys, Gln or Arg; Xaa27 is Gln or Lea; Xaa30 is Ala, Gln or Arg; Xaa33 is Val or Lys; Xaa34 is Lys, Asn or Arg; Xaa35 is Gly; Xaa36 is Arg, Gly or Lys or amide or absent; Xaa37 is Gly, Ala, Gln, Pro, Lys, amide or is absent; or
d) a peptide comprising or consisting of the sequence according to formula III:
(SEQ ID NO: 60)
Xaa7-Xaa8-Glu-Gly-Thr-Phe-Thr-Ser-Asp-Val-Ser-
Xaa18-Tyr-Leu-Glu-Xaa22-Xaa23-Ala-Ala-Xaa26-Glu-
Phe-Ile-Xaa30-Trp-Leu-Val-Xaa34-Xaa35-Xaa36-
Xaa37,
wherein Xaa7 is L-histidine; Xaa8 is Ala, Gly, Val, Len, Ile or Lys; Xaa18 is Ser, Lys or Arg; Xaa22 is Gly or Glu; Xaa23 is Gln, Glu, Lys or Arg; Xaa26 is Lys, Gln or Arg; Xaa30 is Ala, Gln or Arg; Xaa34 is Lys, Gln or Arg; Xaa35 is Gly; Xaa36 is Arg or Lys, amide or is absent; Xaa37 is Gly, Ala, Gln or Lys, amide or is absent; or
e) or a sequence having at least 80% sequence identity with a sequence of any of sequence according to a) to d); and
component (II) C-terminally of component (II) is selected from a peptide sequence of at least 9 amino acids or a functional fragment or variant thereof.

11. The method according to claim 10, wherein component (II) of the GLP-1 fusion peptide, is selected from:
a) a peptide sequence containing a sequence according to SEQ ID NO: 22 (RRDFPEEVAI), SEQ ID NO: 27 (DFPEEVAI), SEQ ID NO: 28 (RDFPEEVA), SEQ ID NO: 29 (RRDFPEEV), SEQ ID NO: 30 (AADFPEEVAI), SEQ ID NO: 31 (ADFPEEVA), or SEQ ID NO: 32 (AADFPEEV), or a sequence having at least 80% sequence identity with SEQ ID NO: 22, 27, 28, 29, 30, 31 or 32; or
b) a peptide sequence containing a sequence according to SEQ ID NO: 23 (RRDFPEEVAIVEEL) or SEQ ID NO: 24 (RRDFPEEVAIAEEL), or SEQ ID NO: 33 (AADFPEEVAIVEEL) or SEQ ID NO: 34 (AADFPEEVAIAEEL), or a sequence having at least 80% sequence identity with SEQ ID NOs: 23, 24, 33 or 34; or
c) a peptide sequence containing a sequence according to SEQ ID NO: 2 (RRDFPEEVAIVEELG), SEQ ID NO: 3 (RRDFPEEVAIAEELG), SEQ ID NO: 35 (AADFPEEVAIVEELG), or SEQ ID NO: 36 (AADFPEEVAIAEELG), or a sequence having at least 80% sequence identity with SEQ ID NOs: 2, 3, 35 or 36.

12. The method according to claim 10, wherein component (I) and component (II) of the GLP-1 fusion peptide are directly linked or linked via a linker sequence.

13. The method according to claim 10, wherein the GLP-1 fusion peptide contains alternatively or additionally to components (I) and (II) a component (III), wherein component (III) may be linked to the C-terminus of component (I) and/or to the N-terminus of component (I), if components (I) and (III) are present in the fusion protein, or wherein component (III) may be linked to the C-terminus of component (II) and/or to the N-terminus of component (I), if components (I), (II) and (III) are present in the fusion protein.

14. The method according to claim 13, wherein component (III) comprises at least four amino acid residues, at least 10 additional amino acid residues, at least 20 additional amino acid residues, or at least 30 additional amino acid residues; preferably selected from
a) the N-terminal sequence of GLP-2 as in proglucagon, or
b) a GLP-1(5-37, 6-37, or 7-37) sequence, or
c) a peptide comprising the sequence according to formula II:
(SEQ ID NO: 59)
Xaa7-Xaa8-Glu-Gly-Thr-Phe-Thr-Ser-Asp-Xaa16-Ser-
Xaa18-Xaa19-Xaa20-Glu-Xaa22-Xaa23-Ala-Xaa25-
Xaa26-Xaa27-Phe-Ile-Xaa30-Trp-Leu-Xaa33-Xaa34-
Xaa35-Xaa36-Xaa37,
wherein Xaa7 is L-histidine; Xaa8 is Ala, Gly, Val, Len, Ile or Lys; Xaa16 is Val or Leu; Xaa18 is Ser, Lys or Arg; Xaa 19 is Tyr or Gln; Xaa20 is Leu or Met; Xaa22 is Gly or Glu; Xaa23 is Gln, Gln, Lys or Arg; Xaa25 is Ala or Val; Xaa26 is Lys, Glu or Arg; Xaa27 is Gln or Leu; Xaa30 is Ala, Glu or Arg; Xaa33 is Val or Lys; Xaa34 is Lys, Glu, Asn or Arg; Xaa35 is Gly; Xaa36 is Arg, Sly or Lys or amide or absent; Xaa37 is Gly, Ala, Glu, Pro, Lys, amide or is absent; or
d) a peptide comprising a sequence according to formula
(SEQ ID NO: 60)
Xaa7-Xaa8-Glu-Gly-Thr-Phe-Thr-Ser-Asp-Val-Ser-
Xaa18-Tyr-Leu-Glu-Xaa22-Xaa23-Ala-Ala-Xaa26-Glu-
Phe-Ile-Xaa30-Trp-Leu-Val-Xaa34-Xaa35-Xaa36-
Xaa37,
wherein Xaa7 is L-histidine; Xaa8 is Ala, Sly, Val, Leu, Ile or Lys; Xaa18 is Ser, Lys or Arg; Xaa22 is Sly or Gln; Xaa23 is Gln, Glu, Lys or Arg; Xaa26 is Lys, Gln or Arg; Xaa30 is Ala, Gln or Arg; Xaa34 is Lys, Glu or Arg; Xaa35 is Gly; Xaa36 is Arg or Lys, amide or is absent; Xaa37 is Gly, Ala, Glu or Lys, amide or is absent; or
e) or a sequence having at least 80% sequence identity with a sequence of any of sequence according to a) to d); or
f) wherein component (III) contains the sequence of SEQ ID NOs: 4 or 5 or a sequence having at least 80% sequence identity with SEQ ID NOs: 4 or 5.

15. The method according to claim 10, wherein the GLP-1 fusion peptide additionally contains or comprises a carrier protein, in particular transferrin or albumin, as component (IV).

16. The method according to claim 1, wherein the GLP-1 fusion peptide comprises or consists of a peptide sequence selected from the sequence of: SEQ ID NO: 6, SEQ ID NO: 7, SEQ ID NO: 8, SEQ ID NO: 10, SEQ ID NO: 11, SEQ NO: 12, SEQ ID NO: 26, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48, or a sequence having at least 80% sequence identity with SEQ ID NOs: 6, 7, 8, 10, 11, 12, 26, or 37 to 48.

17. The method according to claim 1, wherein the cells in the core of the spherical microcapsules are engineered to additionally secrete a factor selected from the group consisting of anti-apoptotic factors, growth factors, VEGF, erythropoietin (EPO), anti-platelet drugs, anti-coagulant drugs, and anti-thrombotic drugs, and/or secrete endogenous proteins or peptides as paracrine factors that are released through the capsule in therapeutic levels selected from VEGF, IL8, GDNF, NT3, and MCP1.

18. The method according to claim 1, wherein the spherical microcapsules are implanted into the affected area of myocardium of a mammalian AMI or post AMI patient by direct injection into the heart tissue, or by intravascular delivery through the arterioles feeding the affected heart tissue.


SEQUENCE LISTING (66 sequences)

SEQ ID NO: 1 - protein, 31 aa, Artificial Sequence
  Description of Artificial Sequence Synthetic polypeptide
HAEGTFTSDVSSYLEGQAAKEFIAWLVKGRG

SEQ ID NO: 2 - protein, 15 aa, Artificial Sequence
  Description of Artificial Sequence Synthetic peptide
RRDFPEEVAIVEELG

SEQ ID NO: 3 - protein, 15 aa, Artificial Sequence
  Description of Artificial Sequence Synthetic peptide
RRDFPEEVAIAEELG

SEQ ID NO: 4 - protein, 35 aa, Artificial Sequence
  Description of Artificial Sequence Synthetic polypeptide
HADGSFSDEMNTILDNLAARDFINWLIQTKITDRK

SEQ ID NO: 5 - protein, 35 aa, Artificial Sequence
  Description of Artificial Sequence Synthetic polypeptide
HADGSFSDEMSTILDNLATRDFINWLIQTKITDKK

SEQ ID NO: 6 - protein, 79 aa, Artificial Sequence
  Description of Artificial Sequence Synthetic polypeptide
HAEGTFTSDVSSYLEGQAAKEFIAWLVKGRGRRDFPEEVAIAEELGRRHAEGTFTSDVSSYLEGQAAKEFIAWLVKGRG

SEQ ID NO: 7 - protein, 83 aa, Artificial Sequence
  Description of Artificial Sequence Synthetic polypeptide
HAEGTFTSDVSSYLEGQAAKEFIAWLVKGRGRRDFPEEVAIAEELGRRHADGSFSDEMSTILDNLATRDFINWLIQTKITDKK

SEQ ID NO: 8 - protein, 46 aa, Artificial Sequence
  Description of Artificial Sequence Synthetic polypeptide
HAEGTFTSDVSSYLEGQAAKEFIAWLVKGRGRRDFPEEVAIAEELG

SEQ ID NO: 9 - protein, 97 aa, Artificial Sequence
  Description of Artificial Sequence Synthetic polypeptide
MAPAAWLRSAAARALLPPMLLLLLQPPPLLARALPPDVHHLHAERRGPQPWHAALPSSPAPAPATQEAPRPASSLRPPRCGVPDPSDGLSARNRQKR

SEQ ID NO: 10 - protein, 79 aa, Artificial Sequence
  Description of Artificial Sequence Synthetic polypeptide
HAEGTFTSDVSSYLEGQAAKEFIAWLVKGRGRRDFPEEVAIVEELGRRHAEGTFTSDVSSYLEGQAAKEFIAWLVKGRG

SEQ ID NO: 11 - protein, 83 aa, Artificial Sequence
  Description of Artificial Sequence Synthetic polypeptide
HAEGTFTSDVSSYLEGQAAKEFIAWLVKGRGRRDFPEEVAIVEELGRRHADGSFSDEMNTILDNLAARDFINWLIQTKITDRK

SEQ ID NO: 12 - protein, 46 aa, Artificial Sequence
  Description of Artificial Sequence Synthetic polypeptide
HAEGTFTSDVSSYLEGQAAKEFIAWLVKGRGRRDFPEEVAIVEELG

SEQ ID NO: 13 - DNA, 815 bp, Artificial Sequence
  Description of Artificial Sequence Synthetic polynucleotide
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcaggggccgcagggacttccctgaggaggtggccatcgtggaggagctgggccggcgacacgccgagggcaccttcacctccgacgtgagcagctacctggagggccaggccgccaaggagttcatcgcctggctggtgaagggcaggggctgagcgcgc

SEQ ID NO: 14 - DNA, 815 bp, Artificial Sequence; identical to SEQ ID NO: 13
  Description of Artificial Sequence Synthetic polynucleotide

SEQ ID NO: 15 - DNA, 834 bp, Artificial Sequence
  Description of Artificial Sequence Synthetic polynucleotide
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcaggggccgcagggacttccctgaggaggtggccatcgtggaggagctgggccggcgacacgccgacggcagcttcagcgacgagatgaacaccatcctggacaacctggccgcgcgcgacttcatcaactggctgatccagaccaagatcaccgatcggaagtgagcgcgctgatatc

SEQ ID NO: 16 - DNA, 834 bp, Artificial Sequence; identical to SEQ ID NO: 15
  Description of Artificial Sequence Synthetic polynucleotide

SEQ ID NO: 17 - DNA, 780 bp, Artificial Sequence
  Description of Artificial Sequence Synthetic polynucleotide
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcaggggccgcagggacttccctgaggaggtggccatcgtggaggagctgggccggcgacacgccgacggcagcttcagcgacgagatgaacaccatcctggacaacctggccgcgcgctgatatc

SEQ ID NO: 18 - DNA, 720 bp, Artificial Sequence
  Description of Artificial Sequence Synthetic polynucleotide
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcagggacggcagcttcagcgacgagatgaacaccatcctggacaacctggccgcgcgctgatatc

SEQ ID NO: 19 - DNA, 716 bp, Artificial Sequence
  Description of Artificial Sequence Synthetic polynucleotide
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcaggggccgcagggacttccctgaggaggtggccatcgtggaggagctgggctgagcgcgc

SEQ ID NO: 20 - DNA, 716 bp, Artificial Sequence; identical to SEQ ID NO: 19
  Description of Artificial Sequence Synthetic polynucleotide

SEQ ID NO: 21 - protein, 31 aa, Artificial Sequence
  Description of Artificial Sequence Synthetic polypeptide
HGEGTFTSDVSSYLEGQAAKEFIAWLVKGRG

SEQ ID NO: 22 - protein, 10 aa, Artificial Sequence
  Description of Artificial Sequence Synthetic peptide
RRDFPEEVAI

SEQ ID NO: 23 - protein, 14 aa, Artificial Sequence
  Description of Artificial Sequence Synthetic peptide
RRDFPEEVAIVEEL

SEQ ID NO: 24 - protein, 14 aa, Artificial Sequence
  Description of Artificial Sequence Synthetic peptide
RRDFPEEVAIAEEL

SEQ ID NO: 25 - protein, 31 aa, Artificial Sequence
  Description of Artificial Sequence Synthetic polypeptide
HAEGTFTSDVSSYLEGQAAKEFIAWLVKGRX

SEQ ID NO: 26 - DNA, 584 bp, Artificial Sequence
  Description of Artificial Sequence Synthetic polynucleotide
aattcagataattcgatagccccgggcaccatggctcccgctgcatggctgagatctgcggccgcgcgcgccctcctgcccccgatgctgctgctgctgctccagccgccgccgctgctggcccgggctctgccgccggacgtccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggcatgccgaagggacctttaccagcgatgtgagctcttatctggaaggccaggctgccaaggagttcattgcttggctggtgaaaggccggggaaggcgggatttcccagaggaggtggccatcgtggaggagctgggccggcgacatgccgaagggacctttaccagcgatgtgagctcttatctggaaggccaggctgccaaggagttcattgcttggctggtgaaaggccggggatgaattgccaagggcgaattatcagg

SEQ ID NO: 27 - protein, 8 aa, Artificial Sequence
  Description of Artificial Sequence Synthetic peptide
DFPEEVAI

SEQ ID NO: 28 - protein, 8 aa, Artificial Sequence
  Description of Artificial Sequence Synthetic peptide
RDFPEEVA

SEQ ID NO: 29 - protein, 8 aa, Artificial Sequence
  Description of Artificial Sequence Synthetic peptide
RRDFPEEV

SEQ ID NO: 30 - protein, 10 aa, Artificial Sequence
  Description of Artificial Sequence Synthetic peptide
AADFPEEVAI

SEQ ID NO: 31 - protein, 8 aa, Artificial Sequence
  Description of Artificial Sequence Synthetic peptide
ADFPEEVA

SEQ ID NO: 32 - protein, 8 aa, Artificial Sequence
  Description of Artificial Sequence Synthetic peptide
AADFPEEV

SEQ ID NO: 33 - protein, 14 aa, Artificial Sequence
  Description of Artificial Sequence Synthetic peptide
AADFPEEVAIVEEL

SEQ ID NO: 34 - protein, 14 aa, Artificial Sequence
  Description of Artificial Sequence Synthetic peptide
AADFPEEVAIAEEL

SEQ ID NO: 35 - protein, 15 aa, Artificial Sequence
  Description of Artificial Sequence Synthetic peptide
AADFPEEVAIVEELG

SEQ ID NO: 36 - protein, 15 aa, Artificial Sequence
  Description of Artificial Sequence Synthetic peptide
AADFPEEVAIAEELG

SEQ ID NO: 37 - protein, 46 aa, Artificial Sequence
  Description of Artificial Sequence Synthetic polypeptide
HAEGTFTSDVSSYLEGQAAKEFIAWLVKGRGAADFPEEVAIAEELG

SEQ ID NO: 38 - protein, 46 aa, Artificial Sequence
  Description of Artificial Sequence Synthetic polypeptide
HAEGTFTSDVSSYLEGQAAKEFIAWLVKGRGAADFPEEVAIVEELG

SEQ ID NO: 39 - protein, 46 aa, Artificial Sequence
  Description of Artificial Sequence Synthetic polypeptide
HAEGTFTSDVSSYLEGQAAKEFIAWLVKGRGRRDFAEEVAIAEELG

SEQ ID NO: 40 - protein, 46 aa, Artificial Sequence
  Description of Artificial Sequence Synthetic polypeptide
HAEGTFTSDVSSYLEGQAAKEFIAWLVKGRGRRDAAAAVAIAEELG

SEQ ID NO: 41 - protein, 46 aa, Artificial Sequence
  Description of Artificial Sequence Synthetic polypeptide
HAEGTFTSDVSSYLEGQAAKEFIAWLVKGRGAADAAAAVAIAAALG

SEQ ID NO: 42 - protein, 36 aa, Artificial Sequence
  Description of Artificial Sequence Synthetic polypeptide
HAEGTFTSDVSSYLEGQAAKEFIAWLVKGRGRRDFP

SEQ ID NO: 43 - protein, 40 aa, Artificial Sequence
  Description of Artificial Sequence Synthetic polypeptide
HAEGTFTSDVSSYLEGQAAKEFIAWLVKGRGRRDFPEEVA

SEQ ID NO: 44 - protein, 51 aa, Artificial Sequence
  Description of Artificial Sequence Synthetic polypeptide
HAEGTFTSDVSSYLEGQAAKEFIAWLVKGRGRRDFPEEVAIAEELGRRHAC

SEQ ID NO: 45 - protein, 46 aa, Artificial Sequence
  Description of Artificial Sequence Synthetic polypeptide
HAEGTFTSDVSSYLEGQAAKEFIAWLVKGRGRRDFAEEVAIVEELG

SEQ ID NO: 46 - protein, 46 aa, Artificial Sequence
  Description of Artificial Sequence Synthetic polypeptide
HAEGTFTSDVSSYLEGQAAKEFIAWLVKGRGRRDAAAAVAIVEELG

SEQ ID NO: 47 - protein, 46 aa, Artificial Sequence
  Description of Artificial Sequence Synthetic polypeptide
HAEGTFTSDVSSYLEGQAAKEFIAWLVKGRGAADAAAAVAIVAALG

SEQ ID NO: 48 - protein, 51 aa, Artificial Sequence
  Description of Artificial Sequence Synthetic polypeptide
HAEGTFTSDVSSYLEGQAAKEFIAWLVKGRGRRDFPEEVAIVEELGRRHAC

SEQ ID NO: 49 - protein, 4 aa, Artificial Sequence
  Description of Artificial Sequence Synthetic peptide
VAIA

SEQ ID NO: 50 - protein, 4 aa, Artificial Sequence
  Description of Artificial Sequence Synthetic peptide
IAEE

SEQ ID NO: 51 - protein, 4 aa, Artificial Sequence
  Description of Artificial Sequence Synthetic peptide
PEEV

SEQ ID NO: 52 - protein, 4 aa, Artificial Sequence
  Description of Artificial Sequence Synthetic peptide
AEEV

SEQ ID NO: 53 - protein, 4 aa, Artificial Sequence
  Description of Artificial Sequence Synthetic peptide
EELG

SEQ ID NO: 54 - protein, 4 aa, Artificial Sequence
  Description of Artificial Sequence Synthetic peptide
AAAA

SEQ ID NO: 55 - protein, 4 aa, Artificial Sequence
  Description of Artificial Sequence Synthetic peptide
AAVA

SEQ ID NO: 56 - protein, 4 aa, Artificial Sequence
  Description of Artificial Sequence Synthetic peptide
AALG

SEQ ID NO: 57 - protein, 4 aa, Artificial Sequence
  Description of Artificial Sequence Synthetic peptide
DFPE

SEQ ID NO: 58 - protein, 9 aa, Artificial Sequence
  Description of Artificial Sequence Synthetic peptide
XXDFPXXXX

SEQ ID NO: 59 - protein, 31 aa, Artificial Sequence
  Description of Artificial Sequence Synthetic polypeptide
HXEGTFTSDXSXXXEXXAXXXFIXWLXXGXX

SEQ ID NO: 60 - protein, 31 aa, Artificial Sequence
  Description of Artificial Sequence Synthetic polypeptide
HXEGTFTSDVSXYLEXXAAXEFIXWLVXGXX

SEQ ID NO: 61 - protein, 4 aa, Artificial Sequence
  Description of Artificial Sequence Synthetic peptide
RQKR

SEQ ID NO: 62 - protein, 176 aa, Artificial Sequence
  Description of Artificial Sequence Synthetic polypeptide
MAPAAWLRSAAARALLPPMLLLLLQPPPLLARALPPDVHHLHAERRGPQPWHAALPSSPAPAPATQEAPRPASSLRPPRCGVPDPSDGLSARNRQKRHAEGTFTSDVSSYLEGQAAKEFIAWLVKGRGRRDFPEEVAIVEELGRRHAEGTFTSDVSSYLEGQAAKEFIAWLVKGRG

SEQ ID NO: 63 - protein, 180 aa, Artificial Sequence
  Description of Artificial Sequence Synthetic polypeptide
MAPAAWLRSAAARALLPPMLLLLLQPPPLLARALPPDVHHLHAERRGPQPWHAALPSSPAPAPATQEAPRPASSLRPPRCGVPDPSDGLSARNRQKRHAEGTFTSDVSSYLEGQAAKEFIAWLVKGRGRRDFPEEVAIVEELGRRHADGSFSDEMNTILDNLAARDFINWLIQTKITDRK

SEQ ID NO: 64 - protein, 165 aa, Artificial Sequence
  Description of Artificial Sequence Synthetic polypeptide
MAPAAWLRSAAARALLPPMLLLLLQPPPLLARALPPDVHHLHAERRGPQPWHAALPSSPAPAPATQEAPRPASSLRPPRCGVPDPSDGLSARNRQKRHAEGTFTSDVSSYLEGQAAKEFIAWLVKGRGRRDFPEEVAIVEELGRRHADGSFSDEMNTILDNLAAR

SEQ ID NO: 65 - protein, 143 aa, Artificial Sequence
  Description of Artificial Sequence Synthetic polypeptide
MAPAAWLRSAAARALLPPMLLLLLQPPPLLARALPPDVHHLHAERRGPQPWHAALPSSPAPAPATQEAPRPASSLRPPRCGVPDPSDGLSARNRQKRHAEGTFTSDVSSYLEGQAAKEFIAWLVKGRGRRDFPEEVAIVEELG

SEQ ID NO: 66 - protein, 176 aa, Artificial Sequence; identical to SEQ ID NO: 62
  Description of Artificial Sequence Synthetic polypeptide